(19) United States
(12) Patent Application Publication
(10) Pub. No.: US 2021/0353783 A1
(43) Pub. Date: Nov. 18, 2021

(54) IMAGING AGENTS FOR RADIOLABELING EXOGENOUS AND ENDOGENOUS ALBUMIN

(71) Applicant: CENTURION BIOPHARMA CORPORATION, Los Angeles, CA (US)

(72) Inventors: Felix Kratz, Ehrenkirchen (DE); Khalid Abu Ajaj, Berlin (DE); Anna Warnecke, Freiburg (DE); Friederike I. Nollmann, Freiburg (DE); Stephan David Koester, Gundelfingen (DE); Javier Garcia Fernandez, Freiburg (DE); Lara Pes, Freiburg (DE); Steffen Daum, Emmendingen (DE); Johannes Pall Magnusson, Freiburg (DE); Serghei Chercheja, Freiburg (DE); Patricia Perez Galan, Freiburg (DE); Federico Medda, Freiburg (DE)

(21) Appl. No.: 17/262,133

(22) PCT Filed: Jul. 16, 2019
(86) PCT No.: PCT/US2019/042025

§ 371 Date: Jan. 21, 2021

Related U.S. Application Data
(60) Provisional application No. 62/702,081, filed on Jul. 23, 2018.

(51) Int. Cl. A61K 51/04; A61K 51/08

(52) U.S. Cl. CPC A61K 51/0478; A61K 51/081

(57) ABSTRACT

The present disclosure provides albumin-binding radioactive metal complexes and uses thereof, including diagnosing and treating disease.

### CROSS REFERENCE TO PRIOR APPLICATIONS

This application claims priority from U.S. provisional application 62/702,081, filed Jul. 23, 2018, the disclosure of which is incorporated by reference herein in its entirety.

### BACKGROUND OF THE INVENTION

Drug delivery in oncology is an approach that has the potential of increasing the narrow therapeutic index of cytotoxic agents. Suitable macromolecular carriers include antibodies, synthetic polymers and serum proteins (F. Kratz et al. (2008): ChemMedChem, 3:20-53).

An attractive approach is to develop prodrugs of cytotoxic agents that bind covalently to circulating serum albumin serving as a macromolecular carrier upon administration. Albumin or its drug conjugates exhibit a markedly long half-life in the systemic circulation of up to 19 days. Because of 1) an enhanced permeability of vessel walls of malignant, infected or inflamed tissue for macromolecules combined with an impaired lymphatic drainage system, and 2) the expression of albumin-binding proteins on tumor endothelia and within the tumor interstitium, albumin-drug conjugates transport the therapeutically effective substance to the target site where the cytotoxic agent is released in a pH-dependent manner or enzymatically (F. Kratz (2008): J. Control. Release, 132:171-183, F. Kratz, U. Beyer (1998): Drug Delivery, 5: 281-299).

The macromolecular prodrug approach targets the cysteine-34 position of albumin that is located in subdomain IA of human serum albumin (HSA). This cysteine residue is highly conserved in all mammalian species studied except for salmon albumin (D. C. Carter, J. X. Ho, (1994): Adv. Protein. Chem. 45: 153-203; T. Jr. Peters (1985): Adv. Protein. Chem. 37:161-245).

The free HS-group of cysteine-34 is an unusual feature of an extracellular protein. The X-ray structure of the defatted protein structure (pdb-entry 1ao6, Crystal structure of human serum albumin, DOI: 10.2210/pdb1AO6/pdb; Protein Data Bank at Brookhaven; version 1.2 (2011 Jul. 13)) reveals that cysteine-34 is located in a crevice on the surface of the protein that is approximately 10-12 Å deep. When HSA is complexed with long-chain fatty acids as in the X-ray structure in which five molecules of myristic acid are bound (pdb-entry 1bj5, Human serum albumin complexed with myristic acid, DOI: 10.2210/pdb1BJ5/pdb; Protein Data Bank at Brookhaven; version 1.2 (2011 Jul. 13)), the crevice is opened up exposing the HS-group of cysteine-34.

In the blood circulation, albumin is generally complexed with one to three molecules of long-chain fatty acids (D. C. Carter, J. X. Ho, (1994): Adv. Protein. Chem. 45:153-203). Approximately 70% of circulating albumin in the blood stream is mercaptalbumin containing an accessible cysteine-34 which is not blocked by endogenous sulfhydryl compounds such as cysteine or glutathione (i.e. non-mercaptalbumin) (M. Sogami et al. (1985): J. Chromatogr. 332:19-27; T. Etoh et al. (1992): J. Chromatogr. 578:292-296; S. Era (1988): Int. J. Peptide Protein Res. 31:435-442).

Considering that free thiol groups are not found on the majority of circulating serum proteins except for albumin, cysteine-34 of endogenous albumin is a unique amino acid on the surface of a circulating protein (reviewed in F. Kratz (2007): Expert Opin. Investig. Drugs 16: 855-866).

In addition, the concentration of low-molecular weight sulfhydryl compounds in their reduced form, e.g. cysteine, homocysteine, cysteinylglycine or glutathione, in the blood is very low, in the order of 0.15-12 μM (M. A. Mansoor, et al. (1992): Anal. Biochemistry 200:218-229; L. Hagenfeldt, et al. (1978): Clin. Chim. Acta 85:167-173) because these are present either as disulfides or are bound to the cysteine-34 position of albumin. Consequently, the free thiol group at the cysteine-34 position of serum albumin accounts by far for the major amount of the total thiol concentration in blood.

Finally, the HS-group of cysteine-34 of HSA is the most reactive thiol group in human plasma due to the low pK_{SH} of Cys-34 in HSA that is approximately 7.0 compared to 8.5 and 8.9 for cysteine and glutathione, respectively (A. Pedersen, J. Jacobsen, (1980): Eur. J. Biochem. 106:291-295).

Taken together, the HS-group of cysteine-34 of HSA is a unique and accessible functional group of a plasma protein that can be exploited for covalent coupling of a thiol-reactive drug derivative to circulating albumin following parenteral administration.

Kratz and co-workers investigated and developed a therapeutic prodrug concept that exploits endogenous albumin as a drug carrier. In this therapeutic strategy, the prodrug is designed to bind rapidly and selectively to the cysteine-34 position of circulating serum albumin after intravenous administration thereby generating a macromolecular transport form of the drug in situ in the blood.

The (6-maleimidocaproyl)hydrazone derivative of doxorubicin (DOXO-EMCH or aldoxorubicin) has been shown to be rapidly and selectively bound to circulating albumin within a few minutes (F. Kratz et al. (2002): J. Med. Chem. 45:5523-5533). Therapy with DOXO-EMCH dramatically improved the efficacy of doxorubicin in preclinical tumor models. Other albumin-binding prodrugs have also been developed by Kratz and co-workers (reviewed in F. Kratz (2008): J. Controlled Release, 132:171-183). These prodrugs consist of an anticancer drug, the maleimide group as the thiol-binding moiety and an enzymatically cleavable peptide linker. Examples include doxorubicin prodrugs that are cleaved by matrix metalloproteases 2 and 9, cathepsin B, urokinase or prostate-specific antigen (PSA), methotrexate prodrugs that are cleaved by cathepsin B and plasmin, and camptothecin prodrugs that are cleaved by cathepsin B or unidentified proteases. In addition, maleimide derivatives with 5-fluorouracil analogues and platinum(II) complexes have been developed.

DOXO-EMCH (also referred to as aldoxorubicin) has been evaluated in phase 1-3 clinical trials in several cancer types exhibiting 3.5-fold increase in the maximum tolerated dose (MTD) compared to conventional doxorubicin, an altered pharmacokinetic profile including large AUC, a small volume of distribution and low clearance compared to doxorubicin, a lack of cardiotoxicity, and significant benefits in first- and second-line soft tissue sarcoma (M. Seetharam, et al. (2018): Future Oncology, Epub ahead of print, Published Online: 5 Jun. 2018.

For achieving a more effective and personalized application of albumin-binding drugs, there is an unmet medical need to diagnose patients regarding the extent of albumin uptake in pathological sites, especially in the tumor and metastatic lesions of cancer patients. To date, a clinically applicable diagnostic and/or imaging agent that identifies and quantifies the albumin uptake in the pathological lesions of patients does not exist.

It is therefore the object of the present invention to provide new imaging agents for radiolabeling endogenous and exogenous albumin at the cysteine-34 position that in combination with suitable imaging techniques allow for detection of albumin in the body and for determination of the extent of albumin uptake and the distribution in the pathological sites of a patient.

This object is achieved through the embodiments of the present invention described and characterized in the claims and in the description and examples.

### SUMMARY OF THE INVENTION

The present invention provides imaging agents for the radiolabeling of the cysteine-34 position of endogenous or exogenous albumin for subsequent radioimaging, preferably using Single Photon Emission Computed Tomography (SPECT).

The present invention relates to imaging agents that comprises a thiol-binding group, an aliphatic and/or an oligoethylene glycol linker incorporating optionally an aromatic moiety, diethylenetriamine pentaacetic acid as a chelating agent, and a radionuclide (e.g., ¹¹¹Indium, ⁶⁷Gallium, or ^{99m}Technetium).

Embodiments of the present disclosure provide a metal complex having the structure represented by Formula (I) or (II) or (III):

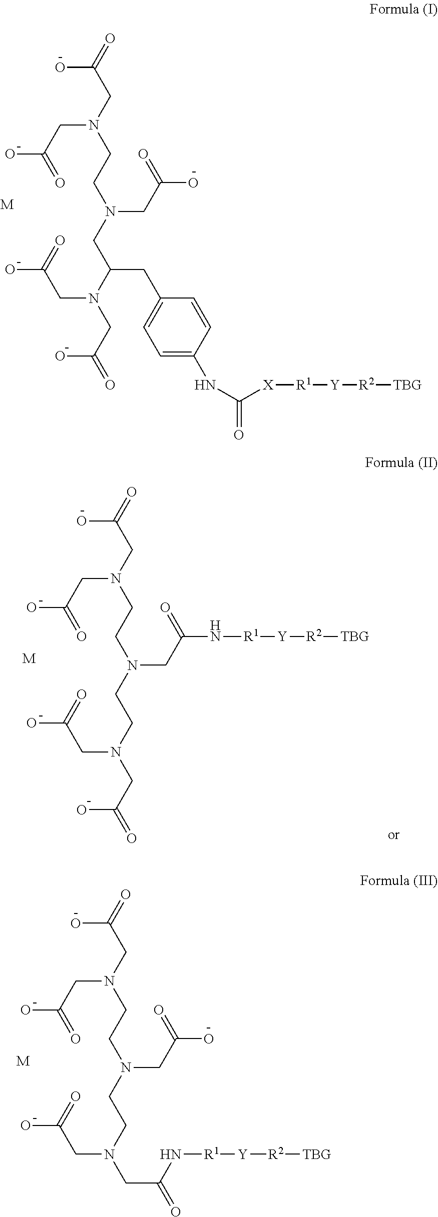

a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
X is absent or selected from —NH—, and —O—;
R¹ is absent or an optionally substituted C₁-C₁₈ alkyl wherein optionally up to six carbon atoms in said C₁-C₁₈ alkyl are each independently replaced with —OCH₂CH₂—;
Y is absent or selected from —O—C(O)—, —C(O)—O—, —NH—C(O)—, —C(O)—NH—, —NH—C(O)—NH—, —O—C(O)—O—, —NH—C(O)—O—, and —O—C(O)—NH—;
R² is an optionally substituted C₁-C₁₈ alkyl wherein optionally up to six carbon atoms in said C₁-C₁₈ alkyl are each independently replaced with —OCH₂CH₂—; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments, the metal complex has the structure of Formula (I):

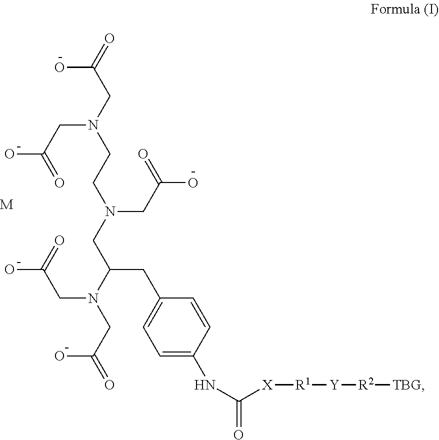

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
X is absent or selected from —NH—, and —O—;
R¹ is absent or an optionally substituted C₁-C₁₈ alkyl wherein optionally up to six carbon atoms in said C₁-C₁₈ alkyl are each independently replaced with —OCH₂CH₂—;
Y is absent or selected from —O—C(O)—, —C(O)—O—, —NH—C(O)—, —C(O)—NH—, —NH—C(O)—NH—, —O—C(O)—O—, —NH—C(O)—O—, and —O—C(O)—NH—;
R² is an optionally substituted C₁-C₁₈ alkyl wherein optionally up to six carbon atoms in said C₁-C₁₈ alkyl are each independently replaced with —OCH₂CH₂—; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments, the metal complex has the structure of Formula (II):

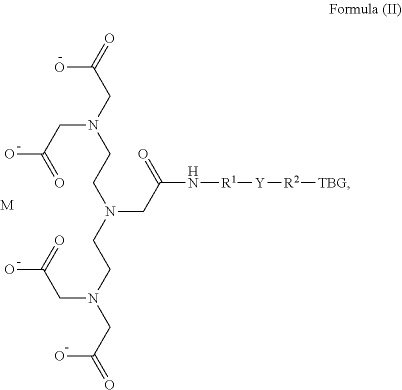

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
R¹ is absent or an optionally substituted C₁-C₁₈ alkyl wherein optionally up to six carbon atoms in said C₁-C₁₈ alkyl are each independently replaced with —OCH₂CH₂—; Y is absent or selected from —O—C(O)—, —C(O)—O—, —NH—C(O)—, —C(O)—NH—, —NH—C(O)—NH—, —O—C(O)—O—, —NH—C(O)—O—, and —O—C(O)—NH—;
R² is an optionally substituted C₁-C₁₈ alkyl wherein optionally up to six carbon atoms in said C₁-C₁₈ alkyl are each independently replaced with —OCH₂CH₂—; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments, the metal complex has the structure of Formula (III):

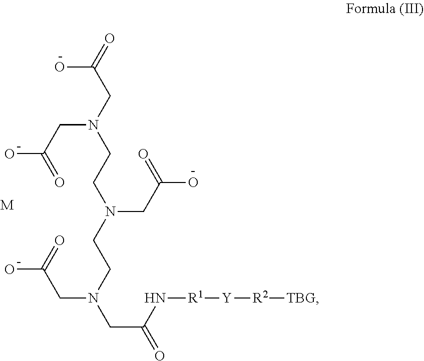

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
R¹ is absent or an optionally substituted C₁-C₁₈ alkyl wherein optionally up to six carbon atoms in said C₁-C₁₈ alkyl are each independently replaced with —OCH₂CH₂—;
Y is absent or selected from —O—C(O)—, —C(O)—O—, —NH—C(O)—, —C(O)—NH—, —NH—C(O)—NH—, —O—C(O)—O—, —NH—C(O)—O—, and —O—C(O)—NH—;
R² is an optionally substituted C₁-C₁₈ alkyl wherein optionally up to six carbon atoms in said C₁-C₁₈ alkyl are each independently replaced with —OCH₂CH₂—; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments, the metal complex has the structure of Formula (IV), (V) or (VI):

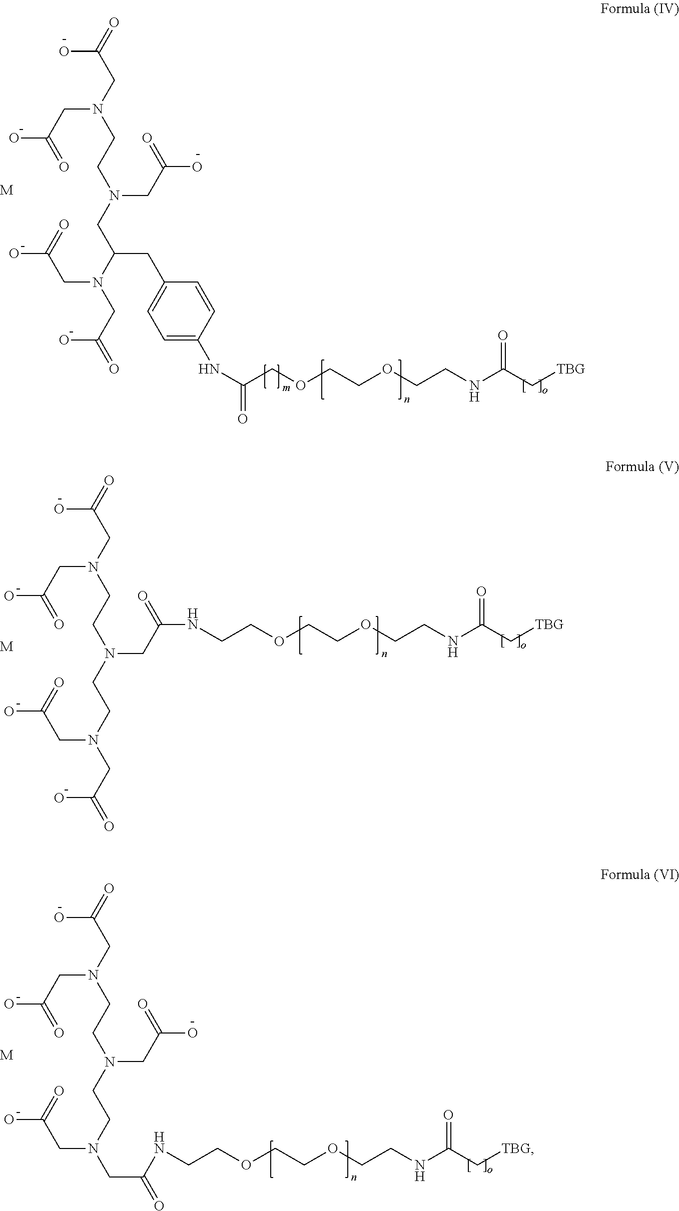

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
m=1 or 2;
n=1-5;
o=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments, the metal complex has the structure of Formula (IV):

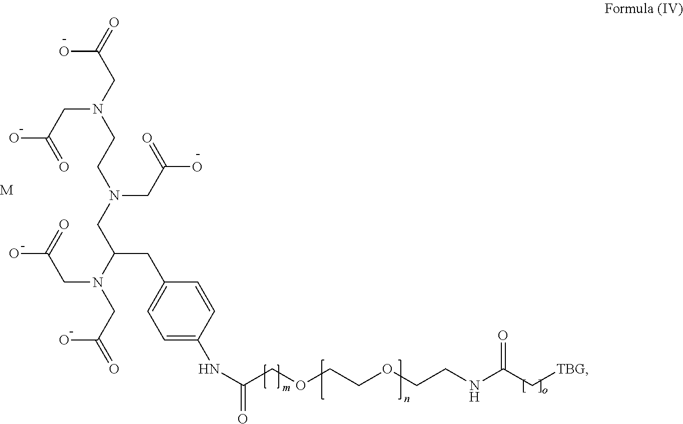

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
m=1 or 2;
n=1-5;
o=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments, the metal complex has the structure of Formula (V):

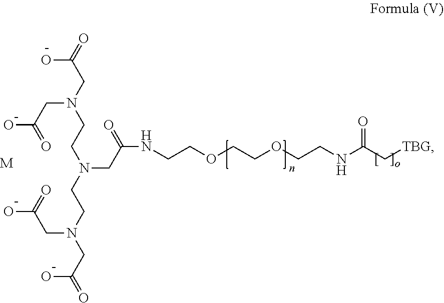

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
n=1-5;
o=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments, the metal complex has the structure of Formula (VI):

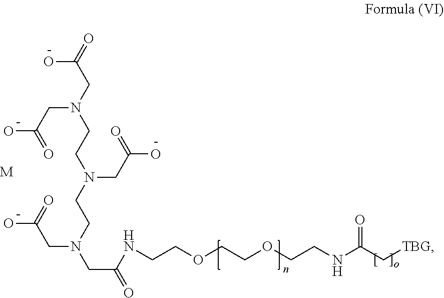

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
n=1-5;
o=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments, the metal complex has the structure of Formula (VII), (VIII) or

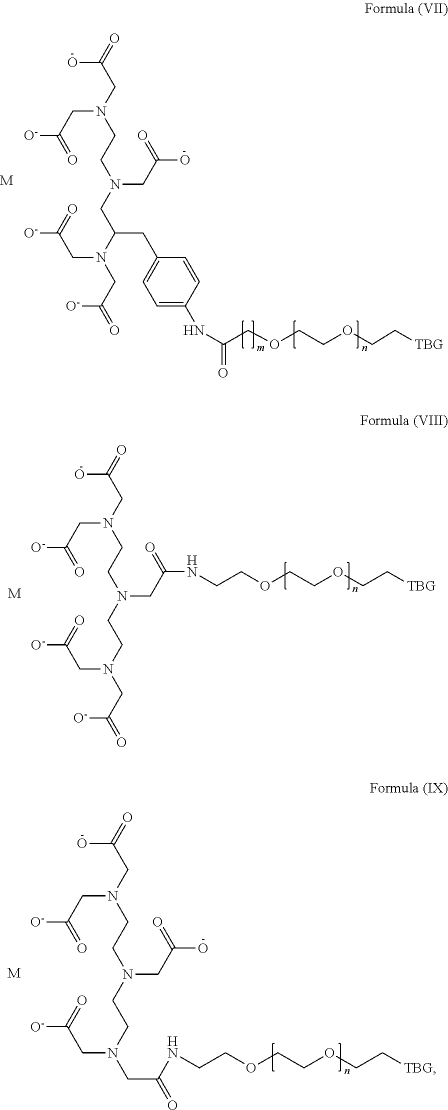

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
m=1 or 2;
n=1-5; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments, the metal complex has the structure of Formula (VII):

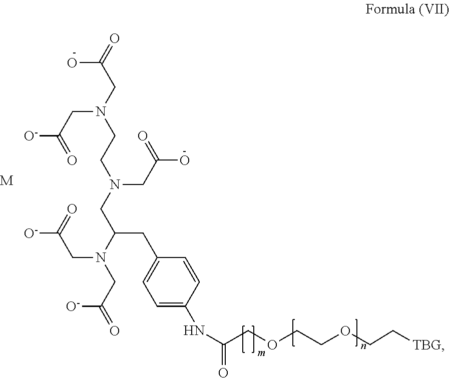

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
m=1 or 2;
n=1-5; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments, the metal complex has the structure of Formula (VIII):

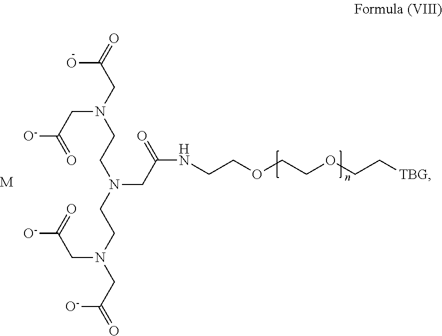

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
n=1-5; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments, the metal complex has the structure of Formula (IX):

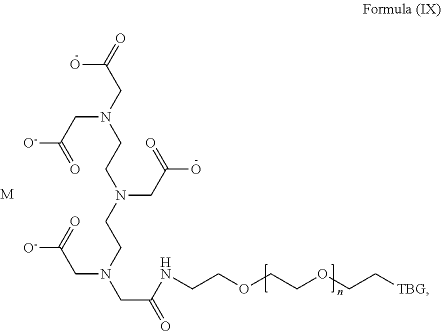

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
n=1-5; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments, the metal complex has the structure of Formula (X), (XI) or (XII):

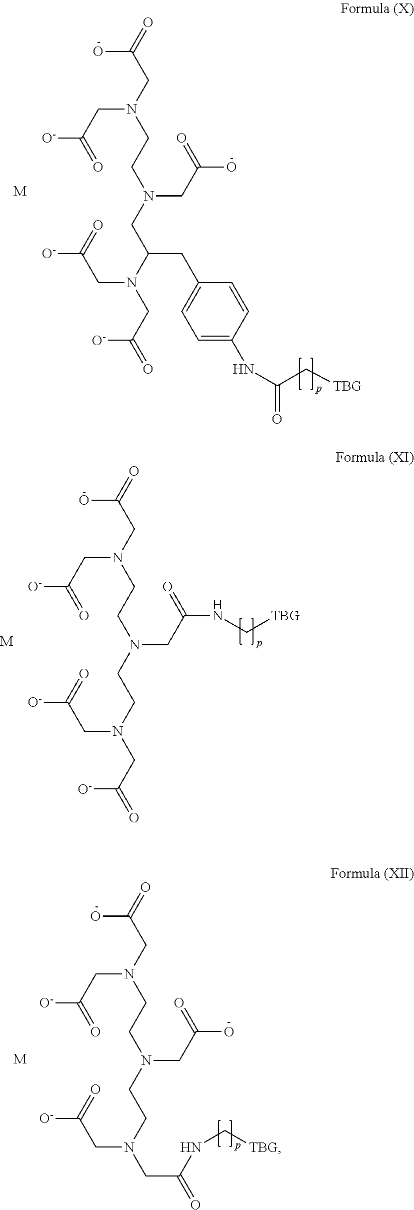

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
p=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments, the metal complex has the structure of Formula (X):

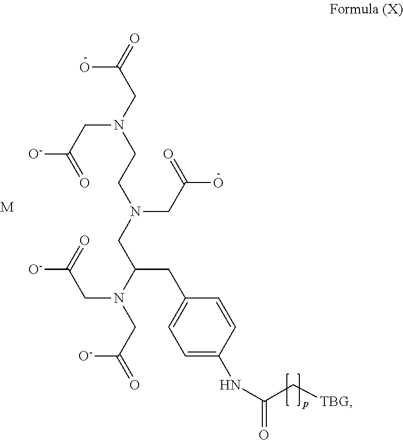

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
p=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene.

In some embodiments, the metal complex has the structure of Formula (XI):

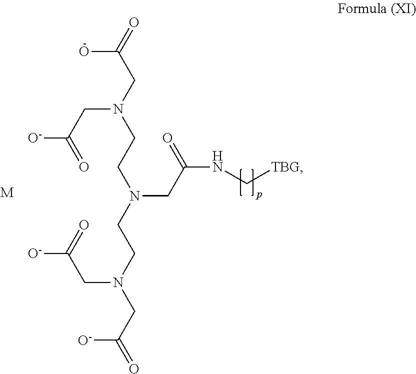

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
p=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments, the metal complex has the structure of Formula (XII):

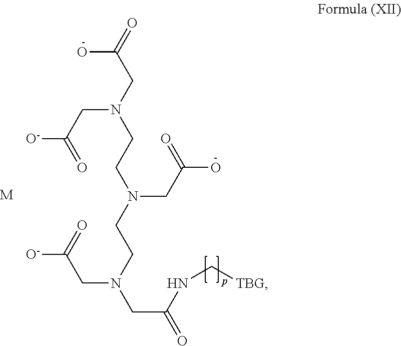

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
p=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments, TBG is an optionally substituted maleimide group. In some embodiments, TBG is

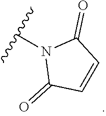 [SMILES: *N1C(=O)C=CC1=O]

In some embodiments, M is ¹¹¹In³⁺.

In some embodiments, the metal complex is selected from:

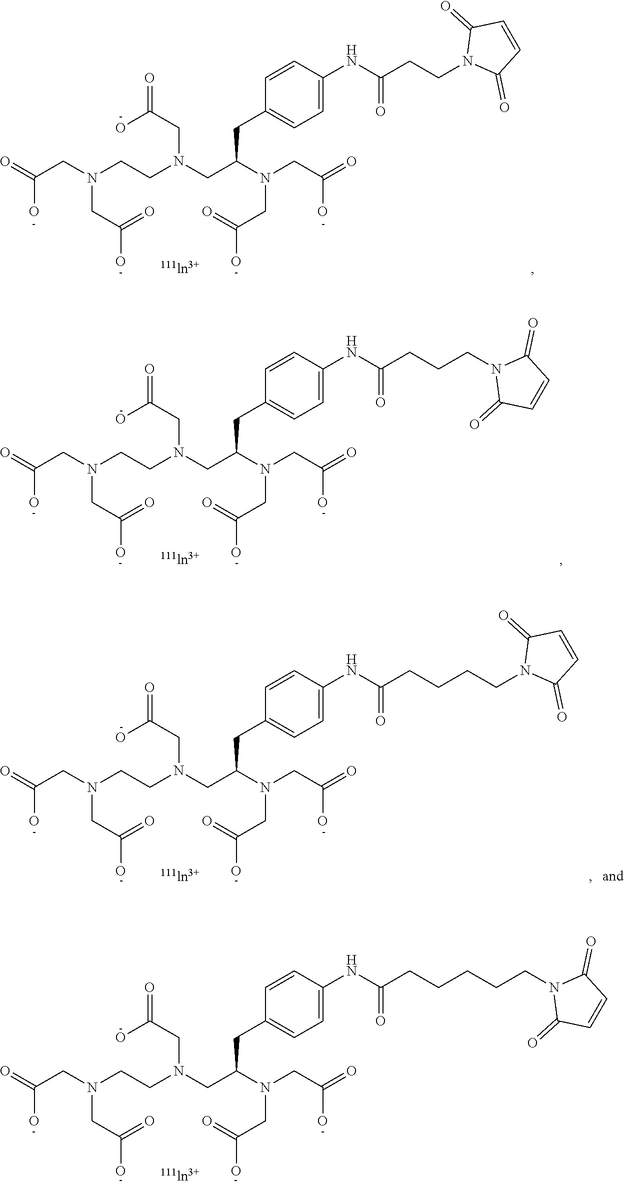

or a conjugate acid thereof, or a pharmaceutically acceptable salt or hydrate thereof.

In some embodiments, the metal complex is selected from:

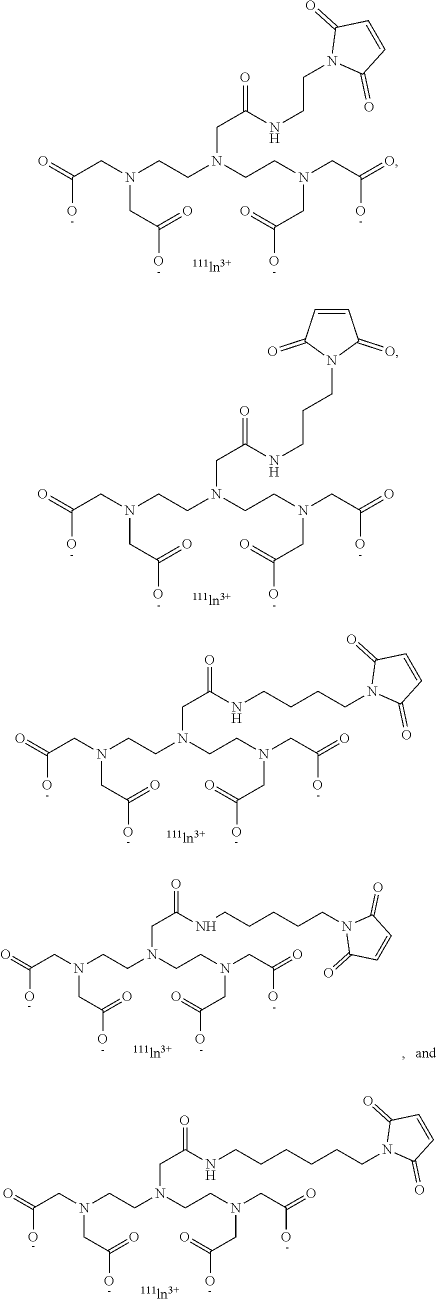

or a conjugate acid thereof, or a pharmaceutically acceptable salt or hydrate thereof.

In some embodiments, the metal complex is selected from:

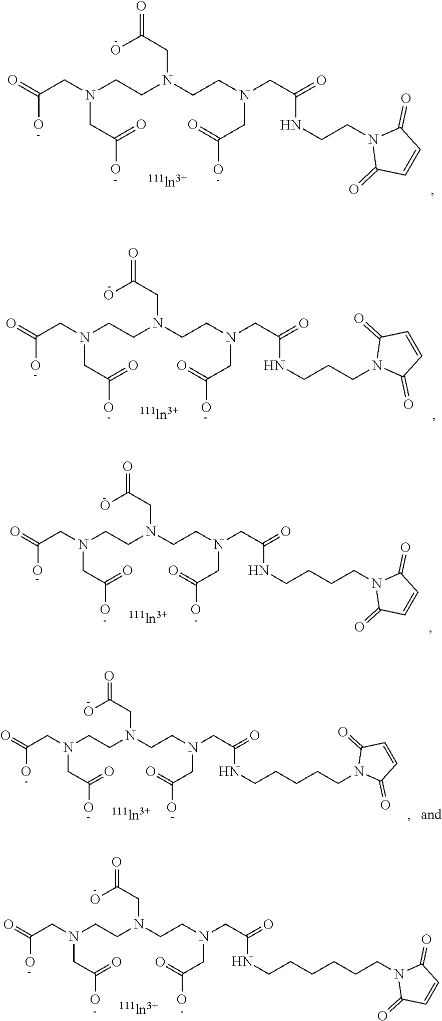

or a conjugate acid thereof, or a pharmaceutically acceptable salt or hydrate thereof.

In some embodiments, the metal complex is selected from:

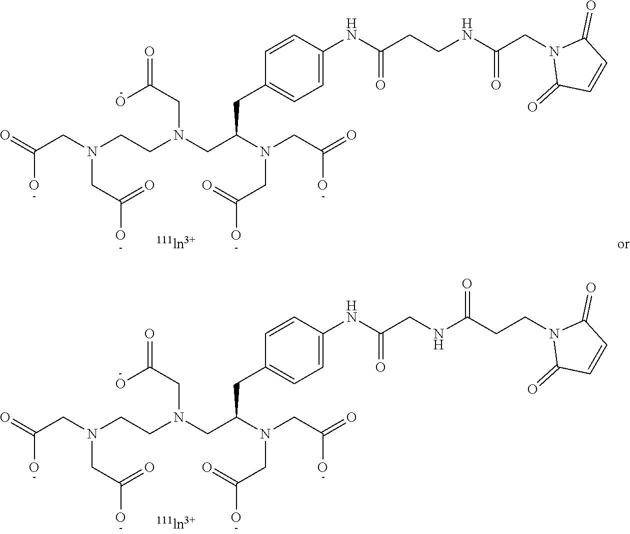

or a conjugate acid thereof, or a pharmaceutically acceptable salt or hydrate thereof.

In some embodiments, the counter cation of the pharmaceutically acceptable salt is selected from: one or two Na⁺, K⁺, or NH₄⁺; or one Ca²⁺ or Mg²⁺.

In some embodiments, the invention provides a pharmaceutical composition comprising a metal complex described herein. In some embodiments, the invention provides a pharmaceutical composition comprising a metal complex described herein and a pharmaceutically acceptable carrier.

In some embodiments, the metal complex covalently binds to the thiol group of cysteine-34 of endogenous or exogenous albumin. In some embodiments, the metal complex binds to the thiol group of cysteine-34 in vivo. In some embodiments, the metal complex binds to the thiol group of cysteine-34 ex vivo. In some embodiments, the binding of the metal complex to albumin is covalent. In other embodiments, the binding of the metal complex to albumin is non-covalent.

In some embodiments, the invention provides a method for diagnosing a disease in a subject, wherein said disease is selected from a cancer, a viral disease, autoimmune disease, acute or chronic inflammatory disease, and a disease caused by bacteria, fungi, or other micro-organisms, comprising administering to the subject a diagnostically effective amount of a metal complex or a pharmaceutical composition as disclosed herein, and subsequently performing SPECT imaging (single-photon emission computed tomography) on said subject. In some embodiments, the disease is cancer.

In some embodiments, the invention provides a method of diagnosing cancer in a subject, the method comprising:

administering a detectable amount of the metal complex or a pharmaceutical composition as disclosed herein, to the subject; and

performing imaging on the subject after administering the metal complex or pharmaceutical composition to detect a signal from a radiolabel of the metal complex, wherein detection of a presence of the signal from the radiolabel in a tissue indicates that the tissue is cancerous, thereby diagnosing cancer in the subject.

In some embodiments, the invention provides a method of diagnosing cancer in a subject, the method comprising:

administering a detectable amount of the metal complex or a pharmaceutical composition as disclosed herein, to the subject; and

performing imaging on the subject after administering the metal complex or pharmaceutical composition, to detect a signal from a radiolabel of the metal complex, wherein detection of a presence of a higher accumulation of the signal from the radiolabel in a tissue in comparison to noncancerous tissue of the same type indicates that the tissue is cancerous, thereby diagnosing cancer in the subject.

A method of diagnosing cancer in a subject, the method comprising:

administering to the subject the metal complex or a pharmaceutical composition as disclosed herein, wherein the metal complex binds to albumin to form a metal complex-albumin conjugate, wherein the metal complex-albumin conjugate accumulates in cancerous tissue; and

performing imaging on the subject after administering the metal complex or pharmaceutical composition, to detect a signal from a radiolabel of the metal complex, wherein detection of a presence of the signal from radiolabel in the tissue indicates that the tissue is cancerous, thereby diagnosing cancer in the subject.

In some embodiments, the invention provides a method of treating cancer in a subject, the method comprising:

administering to the subject the metal complex or a pharmaceutical composition as disclosed herein wherein the metal complex binds to albumin forming a metal complex-albumin conjugate, wherein the metal complex-albumin conjugate accumulates in cancerous tissue;

performing imaging on the subject after administering the metal complex or pharmaceutical composition, to detect a signal from a radiolabel of the metal complex, wherein detection of a presence of the signal from the radiolabel in the tissue indicates that the tissue is cancerous;

diagnosing the subject with cancer in the tissue; and

administering a therapeutically effective amount of a chemotherapeutic agent to the subject.

In some embodiments, the invention provides a method of diagnosing and treating a subject with a cancer responsive to an albumin-binding chemotherapeutic agent, the method comprising:

administering to the subject the metal complex or a pharmaceutical composition as disclosed herein, wherein the metal complex binds to albumin forming a metal complex-albumin conjugate, wherein the metal complex-albumin conjugate accumulates in cancerous tissue;

performing imaging on the subject after administering the metal complex or pharmaceutical composition, to detect a signal from a radiolabel of the metal complex, wherein detection of a presence of the signal from the radiolabel in the tissue indicates that the tissue is cancerous;

diagnosing the subject with cancer that is responsive to treatment with the albumin-binding chemotherapeutic agent; and

administering a therapeutically effective amount of the albumin-binding chemotherapeutic agent to the subject.

In some embodiments, the invention provides a method of diagnosing a subject with a cancer responsive to an albumin-binding chemotherapeutic agent, the method comprising:

administering to the subject the metal complex or a pharmaceutical composition as disclosed herein, wherein the metal complex binds to albumin forming a metal complex-albumin conjugate, wherein the metal complex-albumin conjugate accumulates in cancerous tissue;

performing imaging on the subject after administering the metal complex or pharmaceutical composition, to detect a signal from a radiolabel of the metal complex, wherein detection of a presence of the signal from the radiolabel in the tissue indicates that the tissue is cancerous; and

diagnosing the subject with cancer that is responsive to treatment with the albumin-binding chemotherapeutic agent.

In some embodiments, the invention provides a method of treating a subject with a cancer responsive to an albumin-binding chemotherapeutic agent, the method comprising:

administering to the subject the metal complex or a pharmaceutical composition as disclosed herein, wherein the metal complex binds to albumin forming a metal complex-albumin conjugate, wherein the metal complex-albumin conjugate accumulates in cancerous tissue;

performing imaging on the subject after administering the metal complex or pharmaceutical composition, to detect a signal from a radiolabel of the metal complex, wherein detection of a presence of the signal from the radiolabel in the tissue indicates that the tissue is cancerous;

diagnosing the subject with cancer that is responsive to treatment with the albumin-binding chemotherapeutic agent; and

administering a therapeutically effective amount of the albumin-binding chemotherapeutic agent to the subject.

In some embodiments, the invention provides a method comprising:

administering to a subject the metal complex or a pharmaceutical composition as disclosed herein wherein the metal complex binds to albumin forming a metal complex-albumin conjugate, wherein the metal complex-albumin conjugate accumulates in cancerous tissue;

performing imaging on the subject after administering the metal complex or pharmaceutical composition, to detect a signal from a radiolabel of the metal complex, wherein detection of a presence of the signal from the radiolabel in the tissue indicates that the tissue is cancerous;

diagnosing the subject with cancer in the tissue; and

administering a therapeutically effective amount of an albumin-binding chemotherapeutic agent to the subject.

In some embodiments, the invention provides a method comprising:

administering to a subject the metal complex or a pharmaceutical composition as disclosed herein, wherein the metal complex binds to albumin forming a metal complex-albumin conjugate, wherein the metal complex-albumin conjugate accumulates in cancerous tissue;

performing imaging on the subject after administering the metal complex or pharmaceutical composition, to detect a signal from a radiolabel of the metal complex, wherein detection of a presence of the signal from the radiolabel in the tissue indicates that the tissue is cancerous;

diagnosing the subject with cancer in the tissue;

classifying the subject as being responsive to an albumin-binding chemotherapeutic agent; and

administering a therapeutically effective amount of the albumin-binding chemotherapeutic agent to the subject.

In some embodiments, the invention provides a method for assessing the responsiveness of a subject having cancer to an albumin-binding chemotherapeutic agent comprising:

administering to a subject the metal complex or a pharmaceutical composition as disclosed herein, wherein the metal complex binds to albumin to form a metal complex-albumin conjugate, wherein the metal complex-albumin conjugate accumulates in cancerous tissue;

performing imaging on the subject after administering the metal complex or pharmaceutical composition, to detect a signal from a radiolabel of the metal complex, wherein detection of a presence of the signal from the radiolabel in the tissue indicates that the tissue is cancerous;

diagnosing the subject with cancer in the tissue; and

classifying the subject as responsive to the albumin-binding chemotherapeutic agent.

In some embodiments, the invention provides a method for assessing the susceptibility of a cancer in a subject to an albumin-binding chemotherapeutic agent comprising:

administering to the subject the metal complex or a pharmaceutical composition as disclosed herein, wherein the metal complex binds to albumin to form a metal complex-albumin conjugate, wherein the metal complex-albumin conjugate accumulates in cancerous tissue;

performing imaging on the subject after administering the metal complex or pharmaceutical composition, to detect a signal from a radiolabel of the metal complex, wherein detection of a presence of the signal from the radiolabel in the tissue indicates that the tissue is cancerous;

diagnosing the subject with cancer in the tissue; and

classifying the cancer in the subject as susceptible to the albumin-binding chemotherapeutic agent.

In some embodiments, the invention provides a method for assessing the ability of an albumin-binding chemotherapeutic agent in treating cancer in a subject comprising:

administering to the subject the metal complex or a pharmaceutical composition as disclosed herein, wherein the metal complex binds to albumin to form a metal complex-albumin conjugate, wherein the metal complex-albumin conjugate accumulates in cancerous tissue;

performing imaging on the subject after administering the metal complex or pharmaceutical composition, to detect a signal from a radiolabel of the metal complex, wherein detection of a presence of the signal from the radiolabel in the cancerous tissue indicates that the albumin-binding chemotherapeutic agent is able to treat the cancer in the subject.

In some embodiments, the metal complex is administered as a metal complex-albumin conjugate formed ex vivo. In some embodiments, the metal complex-albumin conjugate is formed by conjugation of albumin to a moiety corresponding to the TBG of the metal complex; followed by chelation of M. In some embodiments, the metal complex-albumin conjugate is formed by chelation of M to form the metal complex; followed by conjugation of albumin to the TBG of the metal complex to form the metal complex-albumin conjugate.

In some embodiments, the cancer is selected from adenocarcinoma, uveal melanoma, acute leukemia, acoustic neuroma, ampullary carcinoma, anal carcinoma, astrocytoma, basalioma, pancreatic cancer, connective tissue tumor, bladder cancer, bronchial carcinoma, non-small cell bronchial carcinoma, breast cancer, Burkitt's lymphoma, corpus carcinoma, CUP syndrome, colon cancer, cancer of the small intestine, ovarian cancer, endometrial carcinoma, gallbladder cancer, gallbladder carcinomas, uterine cancer, cervical cancer, neck, nose and ear tumors, hematological neoplasia, hairy cell leukemia, urethral cancer, skin cancer, gliomas, testicular cancer, Kaposi's sarcoma, laryngeal cancer, bone cancer, colorectal carcinoma, head/neck tumors, colon carcinoma, craniopharyngeoma, liver cancer, leukemia, lung cancer, non-small cell lung cancer, Hodgkin's lymphoma, non-Hodgkin's lymphoma, stomach cancer, colon cancer, medulloblastoma, melanoma, meningioma, kidney cancer, renal cell carcinomas, oligodendroglioma, esophageal carcinoma, osteolytic carcinomas and osteoplastic carcinomas, osteosarcoma, ovarian carcinoma, pancreatic carcinoma, penile cancer, prostate cancer, tongue cancer, ovary carcinoma, and lymph gland cancer.

In some embodiments, the invention provides a kit for assessing the responsiveness of a subject suffering from cancer to an albumin-binding chemotherapeutic agent, wherein the kit comprises a metal complex or pharmaceutical composition disclosed herein. In some embodiments, the invention provides a kit for diagnosing the responsiveness of a subject suffering from cancer to an albumin-binding chemotherapeutic agent, wherein the kit comprises a metal complex disclosed herein. In some embodiments, the invention provides a kit for assessing the responsiveness of a subject suffering from cancer to an albumin-binding chemotherapeutic agent, wherein the kit comprises a pharmaceutical composition disclosed herein.

In some embodiments, the invention provides the use of a metal complex as disclosed herein for the manufacture of a medicament for diagnosing cancer in a subject.

In some embodiments, the invention provides the use of a metal complex as disclosed herein for the manufacture of a medicament for diagnosing a subject with a cancer responsive to an albumin-binding chemotherapeutic agent.

In some embodiments, the invention provides the use of a metal complex as disclosed herein for the manufacture of a medicament for assessing the responsiveness of a subject to an albumin-binding chemotherapeutic agent.

In some embodiments, the invention provides the use of a metal complex as disclosed herein for the manufacture of a medicament for assessing the susceptibility of a cancer in a subject to an albumin-binding chemotherapeutic agent.

In some embodiments, the invention provides a metal complex as disclosed herein for use in diagnosing cancer in a subject.

In some embodiments, the invention provides a metal complex as disclosed herein for use in diagnosing a subject with a cancer responsive to an albumin-binding chemotherapeutic agent.

In some embodiments, the invention provides a metal complex as disclosed herein for use in assessing the responsiveness of a subject to an albumin-binding chemotherapeutic agent.

In some embodiments, the invention provides a metal complex as disclosed herein for use in assessing the susceptibility of a cancer in a subject to an albumin-binding chemotherapeutic agent.

In some embodiments, the invention provides a metal complex as disclosed herein for use in assessing the ability of an albumin-binding chemotherapeutic agent to treat a cancer in a subject.

### BRIEF DESCRIPTION OF THE DRAWINGS

FIG. 1 shows the radiograms for free ¹¹¹In³⁺, ¹¹¹In-C4-DTPA and conjugation of ¹¹¹In-C4-DTPA to albumin in mouse serum. Panel A: Radiogram for free ¹¹¹In³⁺; Panel B: Radiogram for ¹¹¹In-C4-DTPA. Panel C: Radiogram for ¹¹¹In-C4-DTPA-albumin conjugate in mouse serum.

FIG. 2 shows results of the ¹¹¹In-C4-DTPA albumin binding study and complex stability in mouse serum and human serum. Panel A: Rate of ¹¹¹In-C4-DTPA albumin conjugation and complex stability in mouse serum. Panel B: Rate of ¹¹¹In-C4-DTPA albumin conjugation in human serum.

FIG. 3 shows the scheme and radiograms for the radiolabeling of C4-DTPA 3b with ¹¹¹In⁺³. Panel A: Scheme of synthesis of ¹¹¹In-C4-DTPA from C4-DTPA 3b; Panel B: Radiogram for free ¹¹¹In³⁺; Panel C: Radiogram for ¹¹¹In-C4-DTPA.

FIG. 4 shows results for the concentration of radiolabeled albumin with ¹¹¹In-C4-DTPA in blood and plasma over 48 h in healthy nude mice. Panel A: Concentration of albumin-bound ¹¹¹In-C4-DTPA over time in murine blood; Panel B: Concentration of albumin-bound ¹¹¹In-C4-DTPA over time in murine plasma. The concentrations were determined by gamma counting.

FIGS. 5A and 5B show the in vivo SPECT/CT images for patient-derived tumor-bearing mice with bilateral, subcutaneously growing lung LXFL529 xenograft tumors treated with radiolabeled ¹¹¹In-C4-DTPA. FIG. 5A shows the images from mouse #22 and mouse #23. FIG. 5B shows the images from mouse #27 and mouse #29. Circles show native tumors and imaged tumor regions.

FIG. 6 shows representative 3D SPECT/CT images of one tumor-bearing mouse (mouse #22) (LXFL 529 model). Circles show native tumors and imaged tumor regions.

FIG. 7 shows gamma counter results for various tissues from LXFL 529 model 72 h post-dosing with ¹¹¹In-C4-DTPA.

FIGS. 8A and 8B show the SPECT/CT images for patient-derived tumor-bearing mice with bilateral, subcutaneously growing ovarian OVXF 899 tumors treated with radiolabeled ¹¹¹In-C4-DTPA. FIG. 8A shows the images for mouse #1 and mouse #7. FIG. 8B shows the images from mouse #11 and mouse #14. Circles show native tumors and imaged tumor regions.

FIG. 9 shows representative 3D SPECT/CT images of one tumor-bearing mouse (mouse #11) (OVXF 899 model). Circles show native tumors and imaged tumor regions.

FIG. 10 shows gamma counter results for various tissues from OVXF 899 model 72 h post-dosing with ¹¹¹In-C4-DTPA.

### DETAILED DESCRIPTION OF THE INVENTION

In order that the invention described herein may be fully understood, the following detailed description is set forth. While the invention will be described in conjunction with the enumerated embodiments, it will be understood that they are not intended to limit the invention to those embodiments. On the contrary, the invention is intended to cover all alternatives, modifications, and equivalents that can be included within the scope of the present invention as defined by the claims. One skilled in the art will recognize many methods and materials similar or equivalent to those described herein, which could be used in the practice of the present invention.

Unless otherwise defined herein, scientific and technical terms used in this application shall have the meanings that are commonly understood by those of ordinary skill in the art. Generally, nomenclature relating to techniques of chemistry, molecular biology, cell and cancer biology, immunology, microbiology, pharmacology, and protein chemistry, described herein, are those well-known and commonly used in the art. Chemistry terms used herein are used according to conventional usage in the art, as exemplified by “The McGraw-Hill Dictionary of Chemical Terms”, Parker S., Ed., McGraw-Hill, San Francisco, Calif. (1985).

All publications, patents and published patent applications referred to in this application are specifically incorporated by reference herein. In case of conflict, the present specification, including its specific definitions, will control. Unless otherwise specified, it is to be understood that each embodiment disclosed herein may be used alone or in combination with any one or more other embodiments disclosed herein.

The term “herein” means the entire application.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

Throughout the application, where a compound, a metal complex or composition is described as having, including, or comprising, specific components, it is contemplated that such compound, metal complex or composition also may consist essentially of, or consist of, the recited components. Similarly, where methods or processes are described as having, including, or comprising specific process steps, the processes also may consist essentially of, or consist of, the recited processing steps. Further, it should be understood that the order of steps or order for performing certain actions is immaterial so long as the compounds, metal complexes compositions and methods described herein remains operable. Moreover, two or more steps or actions can be conducted simultaneously.

The singular forms “a,” “an,” and “the” include the plural unless the context clearly dictates otherwise.

The term “including” is used to mean “including but not limited to.” “Including” and “including but not limited to” are used interchangeably.

The term “or” as used herein should be understood to mean “and/or”, unless the context clearly indicates otherwise.

As used herein, “about” or “approximately” means within an acceptable error range for the particular value as determined by one of ordinary skill in the art, which will depend in part on how the value is measured or determined, i.e., the limitations of the measurement system.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

The terms “metal complex,” “agent,” “imaging agent”, or “diagnostically effective substance” are used interchangeably and are intended to mean any metal complex which has a diagnostic effect either by itself or after its conversion in the organism in question, and thus also includes the derivatives from these conversions. The term “metal complex” may encompass a metal complex of Formula I-XII, or a conjugate acid thereof, a pharmaceutically acceptable salt thereof or a hydrate thereof.

The term “conjugate acid” of a metal complex disclosed herein, refers to a metal complex of Formula I-XII, wherein one or more carboxylate groups is protonated to form a carboxylic acid.

The terms “compounds,” “metal complex,” “agent,” “imaging agent”, or “diagnostically effective substance” are intended to include compounds, metal complexes, agents, imaging agents and/or substances for which a structure or formula or any derivative thereof has been disclosed in the present invention or a structure or formula or any derivative thereof that has been incorporated by reference. The terms also includes isomers, stereoisomers, geometric isomers, enantiomers, tautomers, solvates, metabolites, conjugate acids, and salts (e.g., pharmaceutically acceptable salts) of a compound, metal complex, agent, imaging agent and/or substance of all the formulae disclosed in the present invention. The terms also include any solvates, hydrates, and polymorphs of any of the foregoing. The specific recitation of “isomers” “stereoisomers,” “geometric isomers,” “enantiomers,” “tautomers,” “solvates,” “metabolites,” “salt” “conjugates,” “conjugate acid,” “conjugate salt,” “solvate,” “hydrate,” or “polymorph” in certain aspects of the invention described in this application shall not be interpreted as an intended omission of these forms in other aspects of the invention where the term “compound” “metal complex,” “agent,” “imaging agent” and/or “substance” is used without recitation of these other forms.

The terms “patient,” “subject,” or “individual” are used interchangeably and refer to either a human or a non-human animal. These terms include mammals such as humans, primates, livestock animals (e.g., bovines, porcines), companion animals (e.g., canines, felines) and rodents (e.g., mice and rats). In certain embodiments, the patient or subject is a human patient or subject, such as a human patient having a condition that needs to be diagnosed.

The term “pharmaceutical composition” refers to a composition suitable for diagnostic use in a subject animal, including but not limited to mammals, e.g., humans, combined with one or more pharmaceutically acceptable carriers, excipients or solvents. Such a composition may also contain diluents, fillers, salts, buffers, stabilizers, solubilizers, protectants and other materials well known in the art. In certain embodiments, a pharmaceutical composition encompasses a composition comprising the active ingredient(s), and the inert ingredient(s) that make up the excipient, carrier or diluent, as well as any product that results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound, conjugate or metal complex of the disclosure and one or more pharmaceutically acceptable excipient(s), carrier(s) and/or diluent(s).

The term “pharmaceutically acceptable carrier” refers to a non-toxic carrier that may be administered to a patient, together with a diagnostically effective substance of this invention, and which does not destroy the diagnostic activity of the imaging agent. The term “excipient” refers to an additive in a formulation or composition that is not a pharmaceutically active ingredient. In certain embodiments, a “pharmaceutically acceptable” substance is suitable for use in contact with cells, tissues or organs of animals or humans without excessive toxicity, irritation, allergic response, immunogenicity or other adverse reactions, in the amount used in the dosage form according to the dosing schedule, and commensurate with a reasonable benefit/risk ratio. In certain embodiments, a “pharmaceutically acceptable” substance that is a component of a pharmaceutical composition is, in addition, compatible with the other ingredient(s) of the composition. In certain embodiments, the terms “pharmaceutically acceptable excipient”, “pharmaceutically acceptable carrier” and “pharmaceutically acceptable diluent” encompass, without limitation, pharmaceutically acceptable inactive ingredients, materials, compositions and vehicles, such as liquid fillers, solid fillers, diluents, excipients, carriers, solvents and encapsulating materials. Carriers, diluents and excipients also include all pharmaceutically acceptable dispersion media, coatings, buffers, isotonic agents, stabilizers, absorption delaying agents, antimicrobial agents, antibacterial agents, antifungal agents, adjuvants, etc. Except insofar as any conventional excipient, carrier or diluent is incompatible with the active ingredient, the present disclosure encompasses the use of conventional excipients, carriers and diluents in pharmaceutical compositions. See, e.g., Remington: The Science and Practice of Pharmacy, 21st Ed., Lippincott Williams & Wilkins (Philadelphia, Pa., 2005); Handbook of Pharmaceutical Excipients, 5th Ed., Rowe et al., Eds., The Pharmaceutical Press and the American Pharmaceutical Association (2005); Handbook of Pharmaceutical Additives, 3rd Ed., Ash and Ash, Eds., Gower Publishing Co. (2007); and Pharmaceutical Preformulation and Formulation, Gibson, Ed., CRC Press LLC (BOCa Raton, Fla., 2004).

The terms “diagnostically effective amount,” or “diagnostically effective dose” refer to an amount effective to diagnose a disease in a patient, e.g., effecting a beneficial and/or desirable identification of pathological sites of a patient suffering from a disease (e.g., cancer). The precise effective amount needed for a subject may depend upon, for example, the subject's size, health and age, the nature and extent of disease, the imaging agent selected for administration, and the mode of administration. The skilled worker can readily determine the effective amount for a given situation by routine experimentation.

The term “therapeutically effective amount” refers to an amount of a compound, agent, or composition, able to treat a disease or symptoms of a disease, lessen the severity of a disease, attenuate symptoms of a disease, put a disease into remission, halt the progression of a disease, or prevent worsening of the disease or symptoms.

As used herein, the terms “diagnosing,” “diagnosis” and variations thereof refers to the identification of a molecular or pathological state, disease or condition, such as the identification of cancer or refers to identification of a cancer patient who may benefit from a particular treatment regimen. In one embodiment, diagnosis refers to the identification of a particular type of tumor.

As used herein, the terms “imaging,” “radioimaging,” and “molecular imaging” refer to an approach for obtaining qualitative, semi-quantitative and/or quantitative images of pathological sites, e.g., tumor and metastatic lesions, relating uptake, accumulation of radiolabeled albumin. An example of such imaging method includes, but is not limited to, Single Photon Emission Computed Tomography (SPECT).

“Administering” or “administration of” a metal complex, an imaging agent or pharmaceutical composition or a chemotherapeutic agent to a subject can be carried out using one of a variety of methods known to those skilled in the art. For example, a metal complex, an imaging agent or pharmaceutical composition can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound, metal complex, or agent can also appropriately be introduced by rechargeable or biodegradable polymeric devices or other devices, e.g., patches and pumps, or formulations, which provide for the extended, slow or controlled release of the compound, metal complex, or agent. Administering can also be performed, for example, once, a plurality of times, and/or over one or more extended periods. Appropriate methods of administering a metal complex, an imaging agent or pharmaceutical composition will also depend, for example, on the age of the subject, whether the subject is active or inactive at the time of administering, whether the subject is cognitively impaired at the time of administering, the extent of the impairment, and the chemical and biological properties of the compound, metal complex, or agent (e.g. solubility, digestibility, bioavailability, stability and toxicity).

The term “substituted” refers to moieties having substituents replacing a hydrogen on one or more carbons of the backbone of a chemical compound or metal complex. It will be understood that “substitution” or “substituted with” includes the implicit proviso that such substitution is in accordance with permitted valence of the substituted atom and the substituent, and that the substitution results in a stable compound or metal complex, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of the invention, the heteroatoms such as nitrogen may have hydrogen substituents, and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, an alkylthio, an acyloxy, a phosphoryl, a phosphate, a phosphonate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety.

“Optional” or “optionally” means that the subsequently described circumstance may or may not occur, so that the application includes instances where the circumstance occurs and instances where it does not. For example, the phrase “optionally substituted” means that a non-hydrogen substituent may or may not be present on a given atom, and, thus, the application includes structures wherein a non-hydrogen substituent is present and structures wherein a non-hydrogen substituent is not present.

Unless specifically stated as “unsubstituted,” references to chemical moieties herein are understood to include substituted variants. For example, reference to an “alkyl” group or moiety implicitly includes both substituted and unsubstituted variants. Examples of substituents on chemical moieties include but is not limited to, halogen, hydroxyl, carbonyl (such as carboxyl, alkoxycarbonyl, formyl, or acyl), thiocarbonyl (such as thioester, thioacetate, or thioformate), alkoxyl, alkylthio, acyloxy, phosphoryl, phosphate, phosphonate, amino, amido, amidine, imine, cyano, nitro, azido, sulfhydryl, alkylthio, sulfate, sulfonate, sulfamoyl, sulfonamido, sulfonyl, heterocyclyl, aralkyl, or aryl or heteroaryl moiety.

The term “alkyl” refers to the radical of saturated aliphatic groups, including straight-chain alkyl groups, and branched-chain alkyl groups. In some embodiments, a straight chain or branched chain alkyl has 30 or fewer carbon atoms in its backbone (e.g., C₁-C₃₀ for straight chains, C₄-C₃₀ for branched chains), and more preferably 20 or fewer. In certain embodiments, alkyl groups are lower alkyl groups, e.g., methyl, ethyl, n-propyl, i-propyl, n-butyl and n-pentyl. Moreover, the term “alkyl” as used throughout the specification, examples, and claims is intended to include both “unsubstituted alkyls” and “substituted alkyls,” the latter of which refers to alkyl moieties having substituents replacing a hydrogen on one or more carbons of the hydrocarbon backbone. In certain embodiments, a straight chain or branched chain alkyl has 30 or fewer carbon atoms in its backbone (e.g., C₁-C₃₀ for straight chains, C₃-C₃₀ for branched chains). In some embodiments, the chain has ten or fewer carbon (C₁-C₁₀) atoms in its backbone. In other embodiments, the chain has six or fewer carbon (C₁-C₆) atoms in its backbone.

Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, an alkylthio, an acyloxy, a phosphoryl, a phosphate, a phosphonate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aryl or heteroaryl moiety.

“Metal complex-albumin conjugate” refers to a compound resulting from the association of a metal complex as described herein with albumin. The association of albumin with the metal complex may be covalent or non-covalent. The covalent connection may be by way of the thiol-binding groups disclosed herein connecting to a cysteine moiety of albumin. In some embodiments, the albumin is covalent connected to the metal complex through cysteine-34.

“Maleimide” refers to the optionally substituted chemical structure:

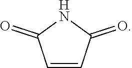

When used as a substituent or a thiol-binding group (TBG) maleimide may have a point of connectivity to the molecule to which it is a substituent of, corresponding to the site of the N—H or C—H bonds of the maleimide.

“Haloacetamide” refers to the structure:

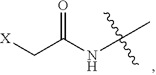

wherein X is a halogen atom (e.g., I, Br, F, for Cl).

“Haloacetate” refers to the structure:

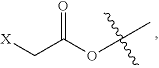

wherein X is a halogen atom (e.g., I, Br, F, or Cl).

“Pyridyldithio” refers to a group or substituent of optionally substituted structure:

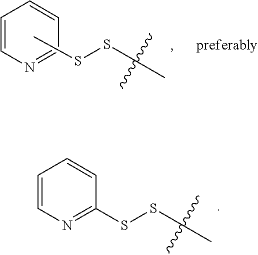

“Isothiocyanate” refers to the structure:

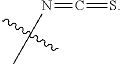 [SMILES: *N=C=S]

“Vinylcarbonyl” refers to an optionally substituted functional group with the formula —CH═CH₂ to which is a carbonyl group is covalently attached having the structure:

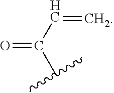 [SMILES: *C(=O)C=C]

“Aziridine” refers to a group that when used as a substituent or TBG, has the optionally substituted structure:

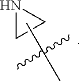

“Acetylene” refers to a group that when used as a substituent or TBG, has the optionally substituted structure:

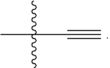 [SMILES: *C#C]

At various places in the present specification substituents of compounds and metal complexes of the disclosure are disclosed in groups or in ranges. It is specifically intended that the disclosure includes each and every individual sub-combination of the members of such groups and ranges. For example, the term “C₁-C₆ alkyl” is specifically intended to individually disclose methyl, ethyl, propyl, isopropyl, n-butyl, sec-butyl, isobutyl, etc.

A “pharmaceutically acceptable salt” is a salt of a compound or metal complex that is suitable for pharmaceutical use, including but not limited to metal salts (e.g., sodium, potassium, magnesium, calcium, etc.), acid addition salts (e.g., mineral acids, carboxylic acids, etc.), and base addition salts (e.g., ammonia, organic amines, etc.). The acid addition salt form of a compound that occurs in its free form as a base can be obtained by treating said free base form with an appropriate acid such as an inorganic acid, for example, a hydrohalic such as hydrochloric or hydrobromic, sulfuric, nitric, phosphoric and the like; or an organic acid, for example, acetic, hydroxyacetic, propanoic, lactic, pyruvic, malonic, succinic, maleic, fumaric, malic, tartaric, citric, methanesulfonic, ethanesulfonic, benzenesulfonic, p-toluenesulfonic, cyclic, salicylic, p-aminosalicylic, pamoic and the like (see, e.g., WO 01/062726. Some pharmaceutically acceptable salts listed by Berge et al., Journal of Pharmaceutical Sciences, 66: 1-19 (1977), incorporated herein by reference in its entirety). Compounds containing acidic protons may be converted into their therapeutically active, non-toxic base addition salt form, e.g. metal or amine salts, by treatment with appropriate organic and inorganic bases. Appropriate base salt forms include, for example, ammonium salts, alkali and earth alkaline metal salts or ions, e.g., lithium, sodium, potassium, magnesium, calcium salts and the like, salts with organic bases, e.g., A-methyl-D-glucamine, hydrabamine salts, and salts with amino acids such as, for example, arginine, lysine and the like. Conversely, said salt forms can be converted into the free forms by treatment with an appropriate base or acid. Compounds or metal complexes and their salts can be in the form of a solvate, which is included within the scope of the present disclosure. Such solvates include for example hydrates, alcoholates and the like (see, e.g., WO 01/062726).

The term “hydrate” refers to salts containing water molecules combined in a definite ratio as an integral part of the crystal that are either bound to a metal center (M) or that have crystallized with the metal complex.

“Chemotherapeutic agent” refers to an agent used in chemotherapy during cancer treatment. Non-limiting examples include alkylating agents, antimetabolites, antimicrotubule agents, topoisomerase inhibitors, and cytotoxic antibiotics.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds,” John Wiley & Sons, Inc., New York, 1994. The compounds, metal complexes, agents, imaging agents and/or substances of the invention can contain asymmetric or chiral centers, and therefore exist in different stereoisomeric forms. It is intended that all stereoisomeric forms, including but not limited to, isomers, diastereomers, enantiomers and atropisomers, as well as mixtures thereof such as racemic mixtures, form part of the present invention. Many organic molecules exist in optically active forms, i.e., they have the ability to rotate the plane of plane-polarized light. In describing an optically active molecule, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A molecule prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

The term “isomer” as used herein includes, but is not limited to, tautomers, cis- and trans-isomers (E (entgegen), Z (zusammen)), R- and S-enantiomers (said R and S notation is used in correspondence with the rules described in Pure Appl. Chem. (1976), 45, 11-30), diastereomers, (D)-isomers, (L)-isomers, stereoisomers, the racemic mixtures thereof, and other mixtures thereof. All such isomers, as well as mixtures thereof, are intended to be included in this invention. Tautomers, while not explicitly indicated in the formulae described herein, are intended to be included within the scope of the present invention.

The term “stereoisomer” refers to molecules that have identical chemical constitution and connectivity, but different orientations of their atoms in space that cannot be interconverted by rotation about single bonds.

The term “diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers can separate under high resolution analytical procedures such as crystallization, electrophoresis and chromatography.

The term “enantiomers” refer to two stereoisomers of a molecule that are non-superimposable mirror images of one another.

The term “tautomer” or “tautomeric form” refers to structural isomers of different energies that are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

### Metal Complexes

One aspect of the present disclosure provides a metal complex having the structure represented by Formula (I) or (II) or (III):

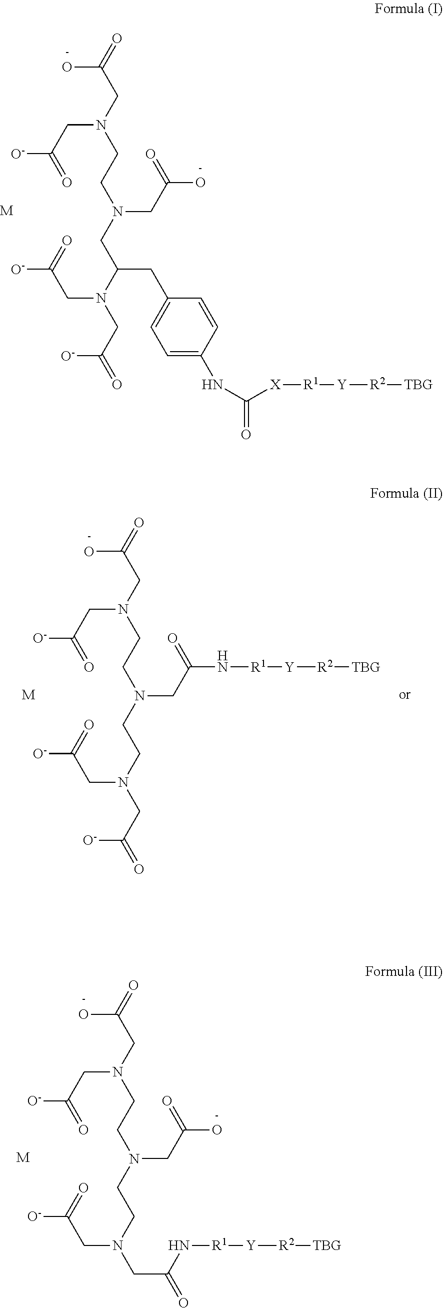

a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
X is absent or selected from —NH—, and —O—;
R¹ is absent or an optionally substituted C₁-C₁₈ alkyl wherein optionally up to six carbon atoms in said C₁-C₁₈ alkyl are each independently replaced with —OCH₂CH₂—;
Y is absent or selected from —O—C(O)—, —C(O)—O—, —NH—C(O)—, —C(O)—NH—, —NH—C(O)—NH—, —O—C(O)—O—, —NH—C(O)—O—, and —O—C(O)—NH—;
R² is an optionally substituted C₁-C₁₈ alkyl wherein optionally up to six carbon atoms in said C₁-C₁₈ alkyl are each independently replaced with —OCH₂CH₂—; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments, the metal complex has the structure represented by Formula (I):

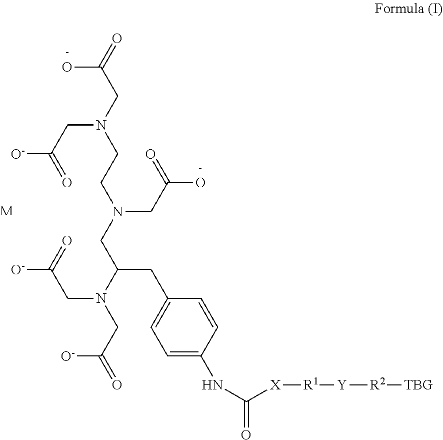

a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
X is absent or selected from —NH—, and —O—;
R¹ is absent or an optionally substituted C₁-C₁₈ alkyl wherein optionally up to six carbon atoms in said C₁-C₁₈ alkyl are each independently replaced with —OCH₂CH₂—;
Y is absent or selected from —O—C(O)—, —C(O)—O—, —NH—C(O)—, —C(O)—NH—, —NH—C(O)—NH—, —O—C(O)—O—, —NH—C(O)—O—, and —O—C(O)—NH—;
R² is an optionally substituted C₁-C₁₈ alkyl wherein optionally up to six carbon atoms in said C₁-C₁₈ alkyl are each independently replaced with —OCH₂CH₂—; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments, X is absent. In some embodiments, X and R¹ are absent. In some embodiments, X, Y, and R¹ are absent. In some embodiments, R² is an optionally substituted C₂-C₅ alkyl. In some embodiments, R² is C₂-C₅ alkyl. In some embodiments, X is absent, R¹ is absent, Y is absent, and R² is an optionally substituted C₂-C₅ alkyl. In some embodiments, X is absent, R¹ is absent, Y is absent, and R² is C₂-C₅ alkyl. In some embodiments, X is absent, R¹ is absent, Y is absent, R² is an optionally substituted C₂-C₅ alkyl, and TBG is an optionally substituted maleimide group. In some embodiments, X is absent, R¹ is absent, Y is absent, R² is C₂-C₅ alkyl, and TBG is a maleimide group. In some embodiments, M is ¹¹¹In³⁺. In some embodiments, M is ⁶⁷Ga³⁺. In some embodiments, M is ^{99m}Tc⁴⁺; In some embodiments, M is ^{99m}Tc³⁺. In some embodiments, X is absent, R¹ is absent, Y is absent, R² is an optionally substituted C₂-C₅ alkyl, M is ¹¹¹In³⁺, and TBG is an optionally substituted maleimide group. In some embodiments, X is absent, R¹ is absent, Y is absent, R² is C₂-C₅ alkyl, M is ¹¹¹In³⁺, and TBG is a maleimide group. In some embodiments, X is absent, R¹ is absent, Y is absent, R² is an optionally substituted C₂-C₅ alkyl, M is ⁶⁷Ga³⁺, and TBG is an optionally substituted maleimide group. In some embodiments, X is absent, R¹ is absent, Y is absent, R² is C₂-C₅ alkyl, M is ⁶⁷Ga³⁺, and TBG is a maleimide group. In some embodiments, X is absent, R¹ is absent, Y is absent, R² is an optionally substituted C₂-C₅ alkyl, M is ^{99m}Tc⁴⁺, and TBG is an optionally substituted maleimide group. In some embodiments, X is absent, R¹ is absent, Y is absent, R² is C₂-C₅ alkyl, M is ^{99m}Tc⁴⁺, and TBG is a maleimide group. In some embodiments, X is absent, R¹ is absent, Y is absent, R² is an optionally substituted C₂-C₅ alkyl, M is ^{99m}Tc³⁺, and TBG is an optionally substituted maleimide group. In some embodiments, X is absent, R¹ is absent, Y is absent, R² is C₂-C₅ alkyl, M is ^{99m}Tc³⁺, and TBG is a maleimide group.

In some embodiments, R¹ is optionally substituted C₁-C₂ alkyl. In some embodiments, R¹ is C₁-C₂ alkyl. In some embodiments, Y is —NH—C(O)—. In some embodiments, X is absent, R¹ is optionally substituted C₁-C₂ alkyl, Y is —NH—C(O)—, and R² is an optionally substituted C₁-C₂ alkyl. In some embodiments, X is absent, R¹ is C₁-C₂ alkyl, Y is —NH—C(O)—, and R² is C₁-C₂ alkyl. In some embodiments, X is absent, R¹ is optionally substituted C₁-C₂ alkyl, Y is —NH—C(O)—, R² is an optionally substituted C₁-C₂ alkyl, and TBG is an optionally substituted maleimide group. In some embodiments, X is absent, R¹ is C₁-C₂ alkyl, Y is —NH—C(O)—, R² is C₁-C₂ alkyl, and TBG is a maleimide group. In some embodiments, X is absent, R¹ is optionally substituted C₁-C₂ alkyl, Y is —NH—C(O)—, R² is an optionally substituted C₁-C₂ alkyl, TBG is an optionally substituted maleimide group, and M is ¹¹¹In³⁺. In some embodiments, X is absent, R¹ is C₁-C₂ alkyl, Y is —NH—C(O)—, R² is C₁-C₂ alkyl, TBG is a maleimide group, and M is ¹¹¹In³⁺. In some embodiments, X is absent, R¹ is optionally substituted C₁-C₂ alkyl, Y is —NH—C(O)—, R² is an optionally substituted C₁-C₂ alkyl, TBG is an optionally substituted maleimide group, and M is ⁶⁷Ga³⁺. In some embodiments, X is absent, R¹ is C₁-C₂ alkyl, Y is —NH—C(O)—, R² is C₁-C₂ alkyl, TBG is a maleimide group, and M is ⁶⁷Ga³⁺. In some embodiments, X is absent, R¹ is optionally substituted C₁-C₂ alkyl, Y is —NH—C(O)—, R² is an optionally substituted C₁-C₂ alkyl, TBG is an optionally substituted maleimide group, and M is ^{99m}Tc⁴⁺. In some embodiments, X is absent, R¹ is C₁-C₂ alkyl, Y is —NH—C(O)—, R² is C₁-C₂ alkyl, TBG is a maleimide group, and M is ^{99m}Tc⁴⁺. In some embodiments, X is absent, R¹ is optionally substituted C₁-C₂ alkyl, Y is —NH—C(O)—, R² is an optionally substituted C₁-C₂ alkyl, TBG is an optionally substituted maleimide group, and M is ^{99m}Tc³⁺. In some embodiments, X is absent, R¹ is C₁-C₂ alkyl, Y is —NH—C(O)—, R² is C₁-C₂ alkyl, TBG is a maleimide group, and M is ^{99m}Tc³⁺.

In some embodiments, the metal complex has the structure represented by Formula (II):

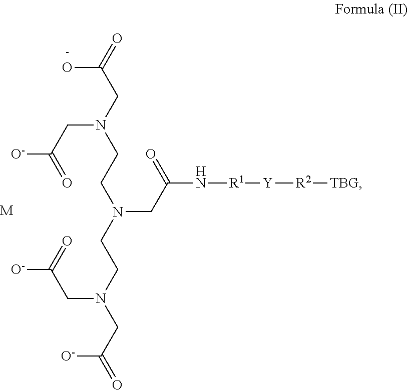

a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺; R¹ is absent or an optionally substituted C₁-C₁₈ alkyl wherein optionally up to six carbon atoms in said C₁-C₁₈ alkyl are each independently replaced with —OCH₂CH₂—; Y is absent or selected from —O—C(O)—, —C(O)—O—, —NH—C(O)—, —C(O)—NH—, —NH—C(O)—NH—, —O—C(O)—O—, —NH—C(O)—O—, and —O—C(O)—NH—; R² is an optionally substituted C₁-C₁₈ alkyl wherein optionally up to six carbon atoms in said C₁-C₁₈ alkyl are each independently replaced with —OCH₂CH₂—; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments, M is ¹¹¹In³⁺. In some embodiments, M is ⁶⁷Ga³⁺. In some embodiments, M is ^{199m}Tc⁴⁺. In some embodiments, M is ^{99m}Tc³⁺. In some embodiments, R¹ is absent. In some embodiments, Y is absent. In some embodiments, R¹ and Y are absent. In some embodiments, R² is an optionally substituted C₁-C₁₈ alkyl. In some embodiments, R² is a C₁-C₁₈ alkyl. In some embodiments, R¹ and Y are absent and R² is an optionally substituted C₁-C₁₈ alkyl. In some embodiments, R¹ and Y are absent and R² is a C₁-C₁₈ alkyl. In some embodiments, R¹ and Y are absent and R² is a C₂-C₆ alkyl.

In some embodiments, TBG is an optionally substituted maleimide group. In some embodiments, TBG is a maleimide group. In some embodiments, TBG is the maleimide group:

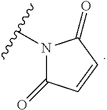 [SMILES: *N1C(=O)C=CC1=O]

In some embodiments, R¹ and Y are absent, R² is a C₂-C₆ alkyl, and TBG is the maleimide group:

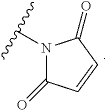 [SMILES: *N1C(=O)C=CC1=O]

In some embodiments, the metal complex has the structure represented by Formula (III):

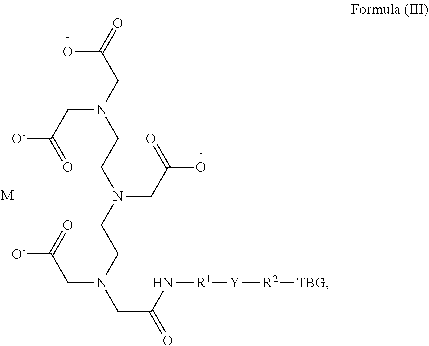

a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
R¹ is absent or an optionally substituted C₁-C₁₈ alkyl wherein optionally up to six carbon atoms in said C₁-C₁₈ alkyl are each independently replaced with —OCH₂CH₂—;
Y is absent or selected from —O—C(O)—, —C(O)—O—, —NH—C(O)—, —C(O)—NH—, —NH—C(O)—NH—, —O—C(O)—O—, —NH—C(O)—O—, and —O—C(O)—NH—;
R² is an optionally substituted C₁-C₁₈ alkyl wherein optionally up to six carbon atoms in said C₁-C₁₈ alkyl are each independently replaced with —OCH₂CH₂—; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments, M is ¹¹¹In³⁺. In some embodiments, M is ⁶⁷Ga³⁺. In some embodiments, M is ^{199m}Tc⁴⁺. In some embodiments, M is ^{99m}Tc³⁺. In some embodiments, R¹ is absent. In some embodiments, Y is absent. In some embodiments, R¹ and Y are absent. In some embodiments, R² is an optionally substituted C₁-C₁₈ alkyl. In some embodiments, R² is a C₁-C₁₈ alkyl. In some embodiments, R² is an optionally substituted C₂-C₆ alkyl. In some embodiments, R² is a C₂-C₆ alkyl. In some embodiments, R¹ and Y are absent and R² is an optionally substituted C₁-C₁₈ alkyl. In some embodiments, R¹ and Y are absent and R² is a C₁-C₁₈ alkyl. In some embodiments, R¹ and Y are absent and R² is an optionally substituted C₂-C₆ alkyl. In some embodiments, R¹ and Y are absent and R² is a C₂-C₆ alkyl.

In some embodiments, TBG is an optionally substituted maleimide group. In some embodiments, TBG is a maleimide group. In some embodiments, TBG is a maleimide group wherein TBG connects to the rest of the metal complex by maleimide's nitrogen. In some embodiments, TBG is the maleimide group:

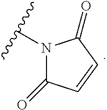 [SMILES: *N1C(=O)C=CC1=O]

In some embodiments, R¹ and Y are absent, R² is a C₂-C₆ alkyl, and TBG is the maleimide group:

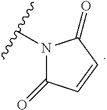 [SMILES: *N1C(=O)C=CC1=O]

In some embodiments, the metal complex has the structure of Formula (IV), (V), or (VI):

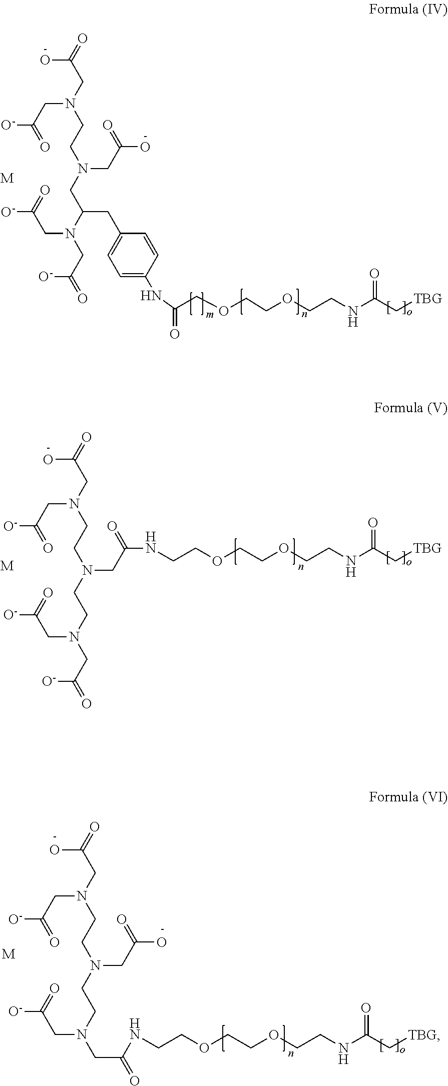

a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
m=1 or 2;
n=1-5;
o=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments, the metal complex has the structure of Formula (IV):

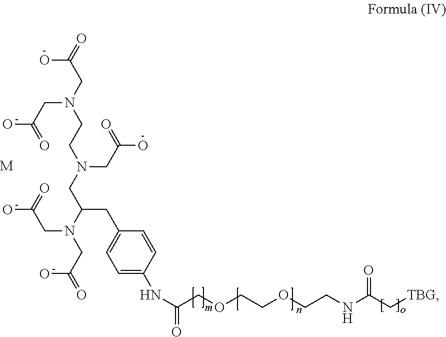

a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
m=1 or 2;
n=1-5;
o=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments M is ¹¹¹In³⁺. In some embodiments, M is ⁶⁷Ga³⁺. In some embodiments M is ^{99m}Tc⁴⁺. In some embodiments, M is ^{99m}Tc³⁺.

In some embodiments, m is 1. In some embodiments, m is 2. In some embodiments, n is 1. In some embodiments, n is 2. In some embodiments, n is 3. In some embodiments, n is 4. In some embodiments, n is 5. In some embodiments, o is 1. In some embodiments, o is 2. In some embodiments, o is 3. In some embodiments, o is 4. In some embodiments, o is 5. In some embodiments, o is 6. In some embodiments, o is 7. In some embodiments, o is 8. In some embodiments, o is 9. In some embodiments, o is 10. In some embodiments, o is 11. In some embodiments, o is 12.

In some embodiments, TBG is an optionally substituted maleimide group. In some embodiments, TBG is a maleimide group. In some embodiments, TBG is the maleimide group:

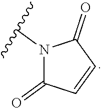 [SMILES: *N1C(=O)C=CC1=O]

In some embodiments, the metal complex has the structure of Formula (V):

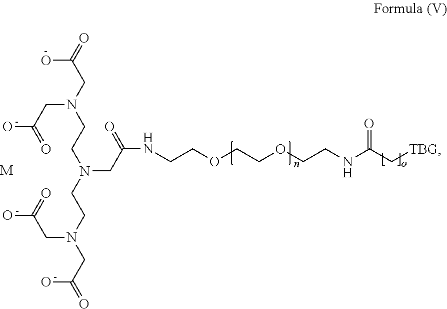

a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
n=1-5;
o=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments M is ¹¹¹In³⁺. In some embodiments, M is ⁶⁷Ga³⁺. In some embodiments, M is ^{99m}Tc⁴⁺. In some embodiments, M is ^{99m}Tc³⁺.

In some embodiments, n is 1. In some embodiments, n is 2. In some embodiments, n is 3. In some embodiments, n is 4. In some embodiments, n is 5. In some embodiments, o is 1. In some embodiments, o is 2. In some embodiments, o is 3. In some embodiments, o is 4. In some embodiments, o is 5. In some embodiments, o is 6. In some embodiments, o is 7. In some embodiments, o is 8. In some embodiments, o is 9. In some embodiments, o is 10. In some embodiments, o is 11. In some embodiments, o is 12.

In some embodiments, TBG is an optionally substituted maleimide group. In some embodiments, TBG is a maleimide group. In some embodiments, TBG is the maleimide group:

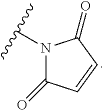 [SMILES: *N1C(=O)C=CC1=O]

In some embodiments, the metal complex has the structure of Formula (VI):

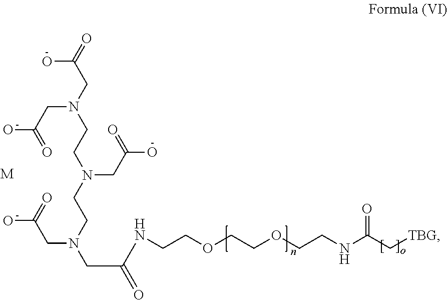

a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
n=1-5;
o=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments M is ¹¹¹In³⁺. In some embodiments, M is ⁶⁷Ga³⁺. In some embodiments, M is ^{199m}Tc⁴⁺. In some embodiments, M is ^{99m}Tc³⁺.

In some embodiments, n is 1. In some embodiments, n is 2. In some embodiments, n is 3. In some embodiments, n is 4. In some embodiments, n is 5. In some embodiments, o is 1. In some embodiments, o is 2. In some embodiments, o is 3. In some embodiments, o is 4. In some embodiments, o is 5. In some embodiments, o is 6. In some embodiments, o is 7. In some embodiments, o is 8. In some embodiments, o is 9. In some embodiments, o is 10. In some embodiments, o is 11. In some embodiments, o is 12.

In some embodiments, TBG is an optionally substituted maleimide group. In some embodiments, TBG is a maleimide group. In some embodiments, TBG is the maleimide group:

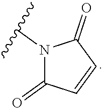 [SMILES: *N1C(=O)C=CC1=O]

In some embodiments, the metal complex has the structure of Formula (VII), (VIII), or (IX):

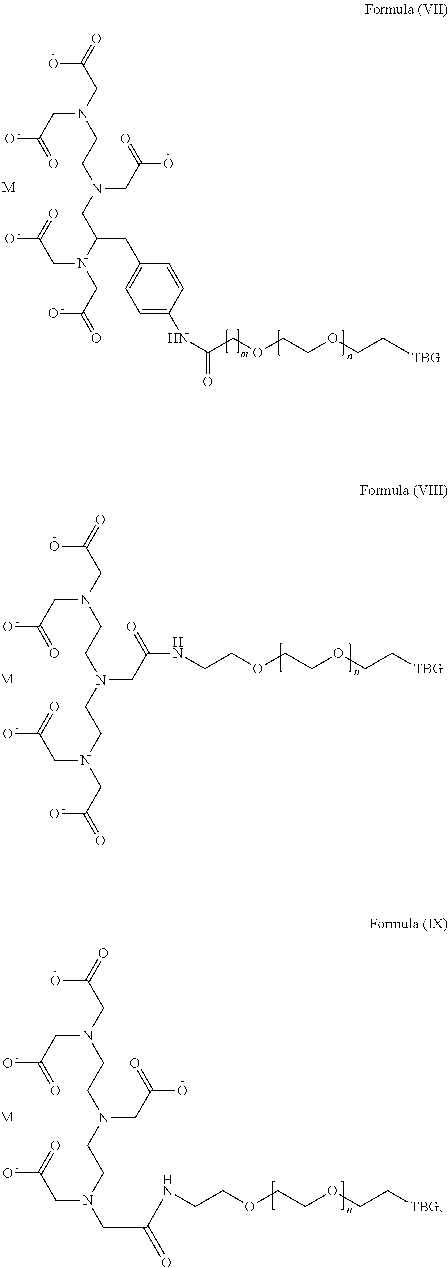

a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
m=1 or 2;
n=1-5; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments, the metal complex has the structure of Formula (VII):

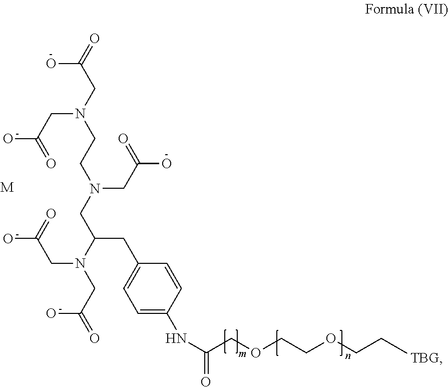

a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
m=1 or 2;
n=1-5; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments M is ¹¹¹In³⁺. In some embodiments, M is ⁶⁷Ga³⁺. In some embodiments, M is ^{199m}Tc⁴⁺. In some embodiments, M is ^{99m}Tc³⁺.

In some embodiments, m is 1. In some embodiments, m is 2. In some embodiments, n is 1. In some embodiments, n is 2. In some embodiments, n is 3. In some embodiments, n is 4. In some embodiments, n is 5.

In some embodiments, TBG is an optionally substituted maleimide group. In some embodiments, TBG is a maleimide group. In some embodiments, TBG is the maleimide group:

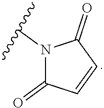 [SMILES: *N1C(=O)C=CC1=O]

In some embodiments, the metal complex has the structure of Formula (VIII):

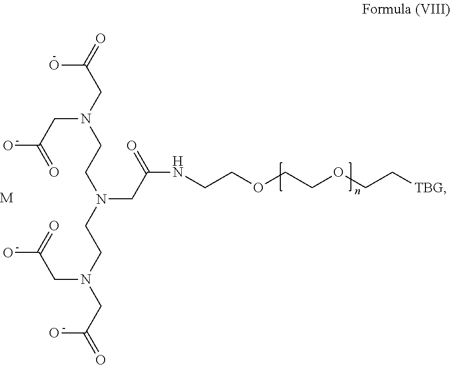

a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
n=1-5; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments M is ¹¹¹In³⁺. In some embodiments, M is ⁶⁷Ga³⁺. In some embodiments, M is ^{199m}Tc⁴⁺. In some embodiments, M is ^{99m}Tc³⁺. In some embodiments, n is 1. In some embodiments, n is 2. In some embodiments, n is 3. In some embodiments, n is 4. In some embodiments, n is 5.

In some embodiments, TBG is an optionally substituted maleimide group. In some embodiments, TBG is a maleimide group. In some embodiments, TBG is a maleimide group:

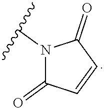 [SMILES: *N1C(=O)C=CC1=O]

In some embodiments, the metal complex has the structure of Formula (IX):

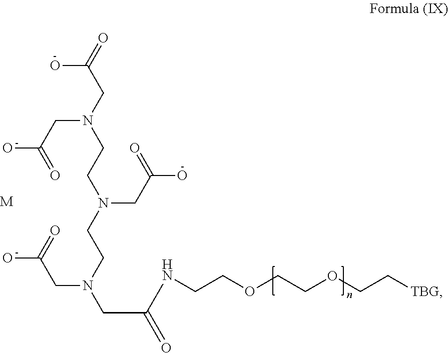

a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
n=1-5; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments M is ¹¹¹In³⁺. In some embodiments, M is ⁶⁷Ga³⁺. In some embodiments, M is ^{199m}Tc⁴⁺. In some embodiments, M is ^{99m}Tc³⁺. In some embodiments, n is 1. In some embodiments, n is 2. In some embodiments, n is 3. In some embodiments, n is 4. In some embodiments, n is 5.

In some embodiments, TBG is an optionally substituted maleimide group. In some embodiments, TBG is a maleimide group. In some embodiments, TBG is the maleimide group:

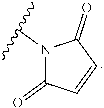 [SMILES: *N1C(=O)C=CC1=O]

In some embodiments, the metal complex has the structure of Formula (X), (XI), or (XII):

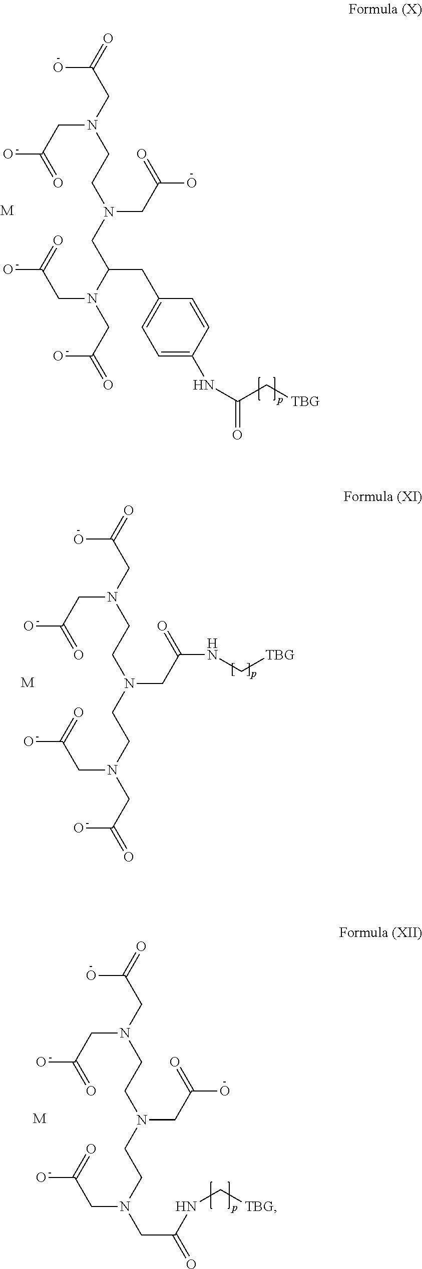

a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
p=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments, TBG is an optionally substituted maleimide group. In some embodiments, TBG is the maleimide group:

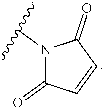 [SMILES: *N1C(=O)C=CC1=O]

In some embodiments, M is ¹¹¹In³⁺. In some embodiments, M is ⁶⁷Ga³⁺. In some embodiments, M is ^{199m}Tc⁴⁺. In some embodiments, M is ^{99m}Tc³⁺.

In some embodiments, the metal complex has the structure of Formula (X):

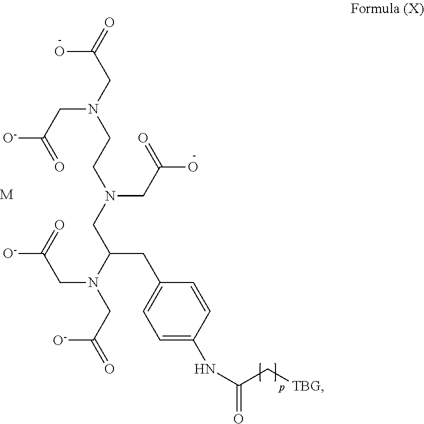

a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
p=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments M is ¹¹¹In³⁺. In some embodiments, M is ⁶⁷Ga³⁺. In some embodiments, M is ^{199m}Tc⁴⁺. In some embodiments, M is ^{99m}Tc³⁺.

In some embodiments, p is 1. In some embodiments, p is 2. In some embodiments, p is 3. In some embodiments, p is 4. In some embodiments, p is 5. In some embodiments, p is 6. In some embodiments, p is 7. In some embodiments, p is 8. In some embodiments, p is 9. In some embodiments, p is 10. In some embodiments, p is 11. In some embodiments p is 12.

In some embodiments, TBG is an optionally substituted maleimide group. In some embodiments, TBG is a maleimide group. In some embodiments, TBG is the maleimide group:

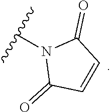 [SMILES: *N1C(=O)C=CC1=O]

In some embodiments, the metal complex has the structure of Formula (XI):

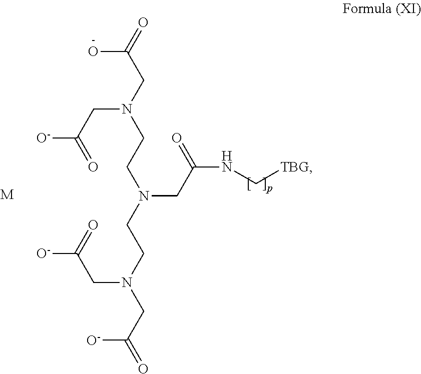

a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
p=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments M is ¹¹¹In³⁺. In some embodiments, M is ⁶⁷Ga³⁺. In some embodiments, M is ^{199m}Tc⁴⁺. In some embodiments, M is ^{99m}Tc³⁺. In some embodiments, p is 1. In some embodiments, p is 2. In some embodiments, p is 3. In some embodiments, p is 4. In some embodiments, p is 5. In some embodiments, p is 6. In some embodiments, p is 7. In some embodiments, p is 8. In some embodiments, p is 9. In some embodiments, p is 10. In some embodiments, p is 11. In some embodiments, p is 12.

In some embodiments, TBG is an optionally substituted maleimide group. In some embodiments, TBG is a maleimide group. In some embodiments, TBG is the maleimide group:

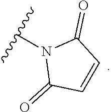 [SMILES: *N1C(=O)C=CC1=O]

In some embodiments, the metal complex has the structure of Formula (XII):

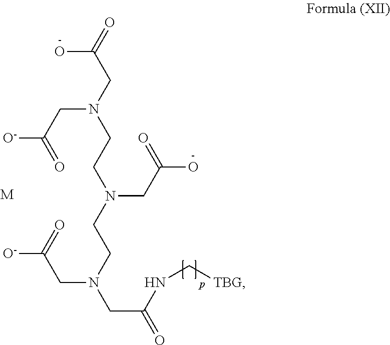

a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
p=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

In some embodiments M is ¹¹¹In³⁺. In some embodiments, M is ⁶⁷Ga³⁺. In some embodiments, M is ^{199m}Tc⁴⁺. In some embodiments, M is ^{99m}Tc³⁺. In some embodiments, p is 1. In some embodiments, p is 2. In some embodiments, p is 3. In some embodiments, p is 4. In some embodiments, p is 5. In some embodiments, p is 6. In some embodiments, p is 7. In some embodiments, p is 8. In some embodiments, p is 9. In some embodiments, p is 10. In some embodiments, p is 11. In some embodiments, p is 12.

In some embodiments, TBG is an optionally substituted maleimide group. In some embodiments, TBG is a maleimide group. In some embodiments, TBG is a maleimide group wherein TBG connects to the rest of the metal complex by maleimide's nitrogen and p is 2-6. In some embodiments, TBG is a maleimide group:

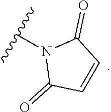 [SMILES: *N1C(=O)C=CC1=O]

In some embodiments, the metal complex is selected from

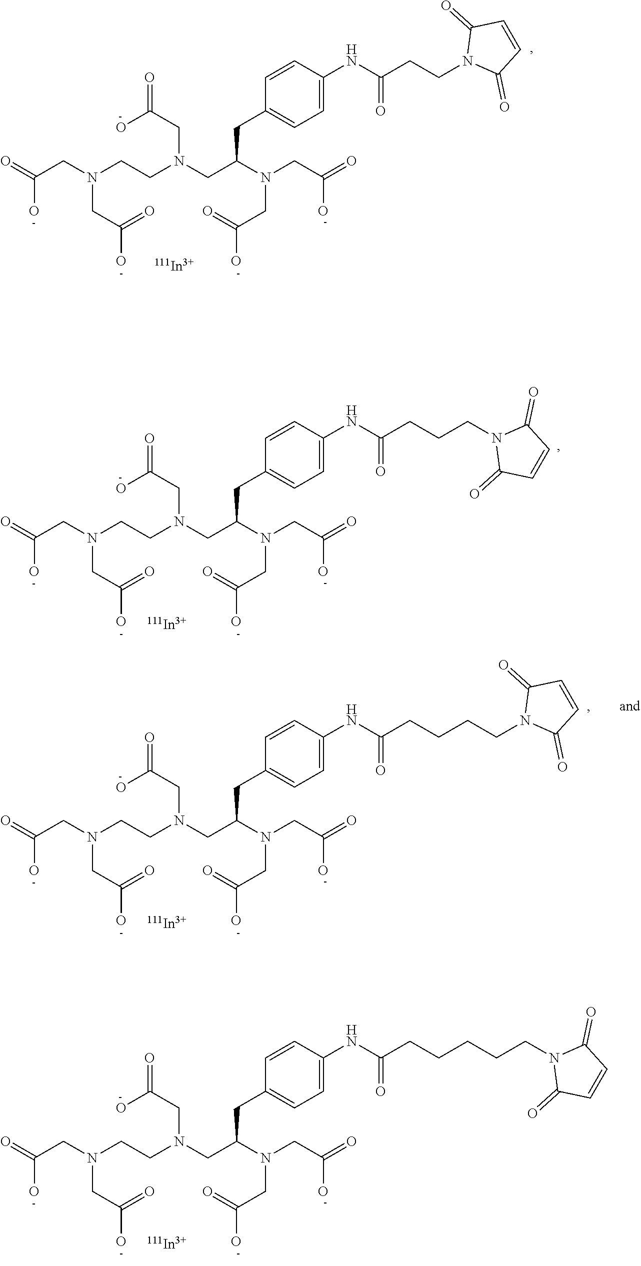

or a conjugate acid thereof, or a pharmaceutically acceptable salt or hydrate thereof.

In some embodiments, the metal complex is selected from

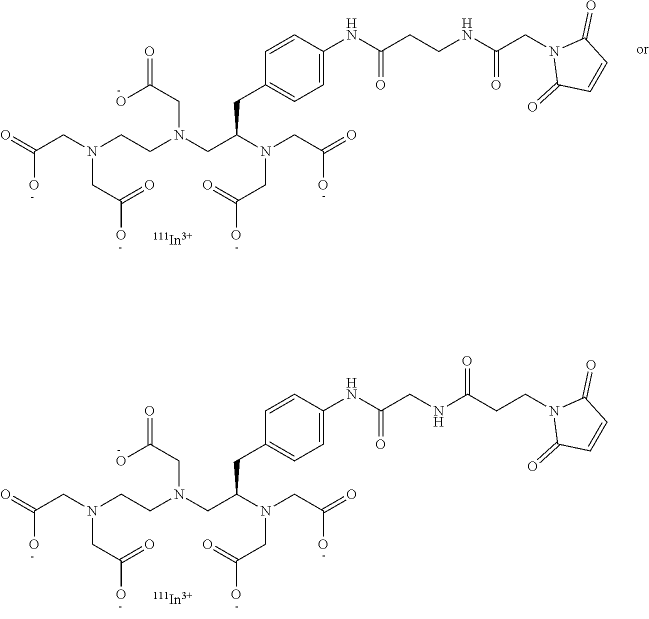

or a conjugate acid thereof, or a pharmaceutically acceptable salt or hydrate thereof.

In some embodiments, the metal complex is

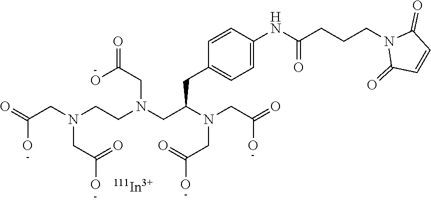

or a conjugate acid thereof, or a pharmaceutically acceptable salt or hydrate thereof.

In some embodiments, the metal complex is selected from

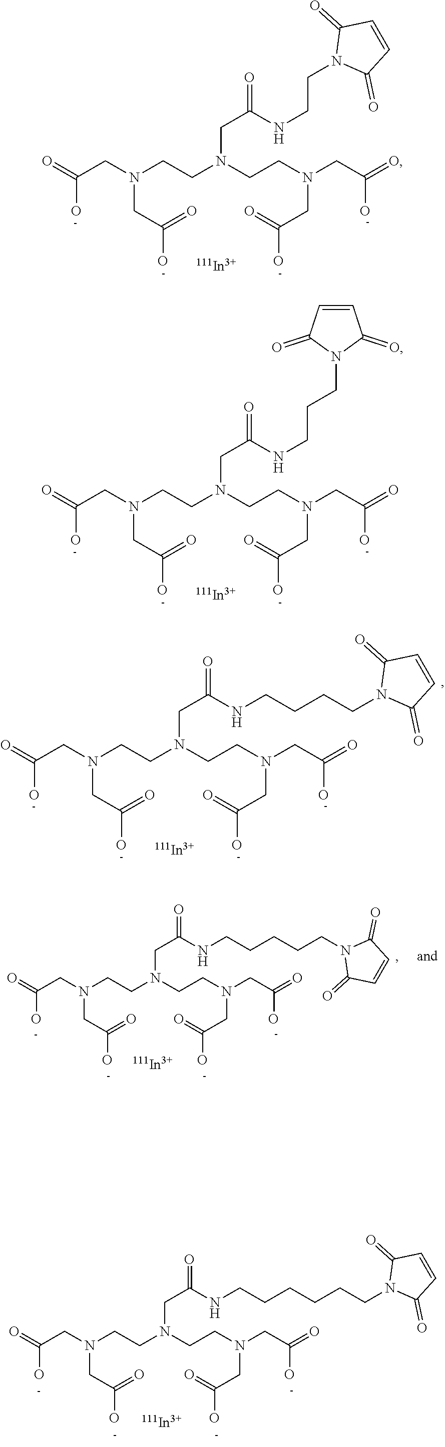

or a conjugate acid thereof, or a pharmaceutically acceptable salt or hydrate thereof.

In some embodiments, the metal complex is selected from

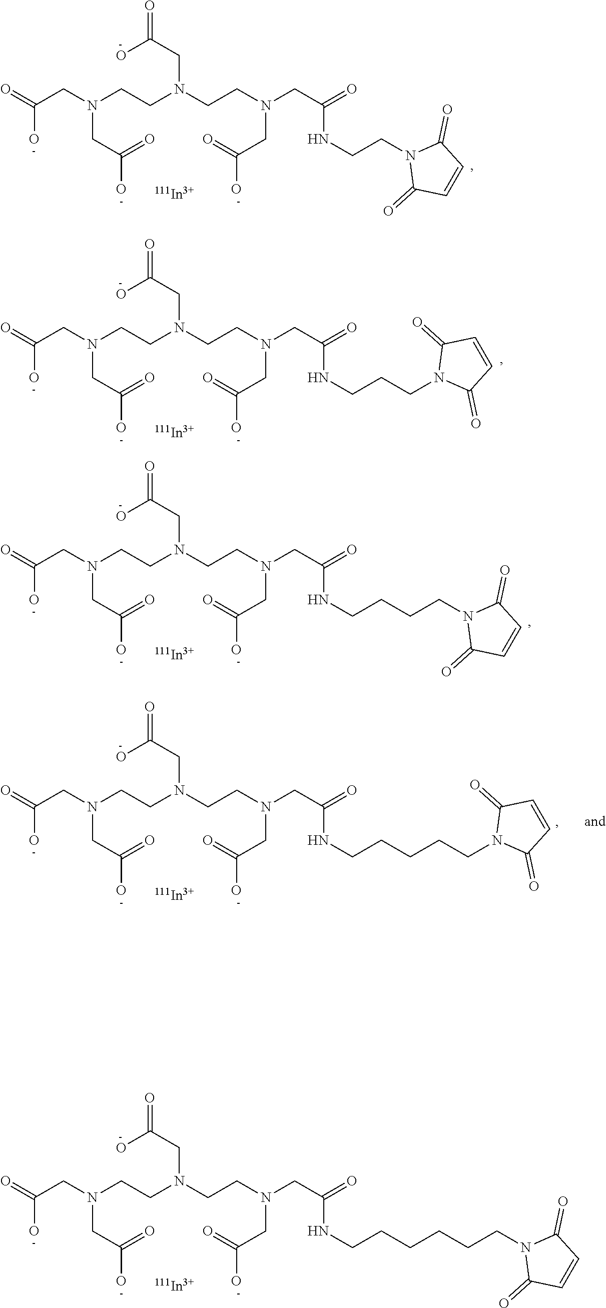

or a conjugate acid thereof, or a pharmaceutically acceptable salt or hydrate thereof.

In some embodiments, the pharmaceutically acceptable salt of a metal complex or conjugate acid thereof described herein, comprises a counter cation selected from one or two Na⁺, K⁺, or NH₄⁺; or one Ca²⁺ or Mg²⁺.

Albumin may be derivatized by any of metal complex disclosed herein (e.g., forming a metal complex-albumin conjugate). In some embodiments albumin is derivatized by a conjugate acid of the metal complex. Derivatizing albumin at different amino acid sites of the protein has a major influence on the pharmacokinetic behavior of albumin. The first drug-albumin conjugate that entered clinical trials was an ex vivo synthesized methotrexate-albumin conjugate in which the glutamate moiety of methotrexate was covalently bound to the epsilon-amino group of lysine residues of human serum albumin in which the ratio of methotrexate:albumin was approximately 1.0. The half-life of this methotrexate-albumin conjugate was estimated to be up to 3 weeks in humans (G. Hartung et al. (1999): Clin. Cancer. Res. 5:753-759). In contrast, the human half-life of the (6-maleimidocaproyl)hydrazone derivative of doxorubicin (DOXO-EMCH, renamed aldoxorubicin) which binds selectively and specifically to the cysteine-34 of endogenous albumin after intravenous administration, was considerably lower, t_{1/2} in man being 20-21 h (M. M. Mita et al. (2015): Invest. New Drugs 33:341-348). Considering that the thiol-binding prodrug, aldoxorubicin, has shown a favorable toxicity profile and improved efficacy over doxorubicin in first- and second-line soft tissue sarcoma (M. Seetharam et al. (2018): Future Oncology, Epub ahead of print published online: 5 Jun. 2018), a suitable imaging agent would therefore have to bind specifically and selectively to the cysteine-34 position of endogenous or exogenous albumin in order to diagnose the uptake of serum albumin modified at this amino acid in pathological sites, in particular in tumor or metastatic lesions. In some embodiments, the association of albumin with the imaging agent is covalent. In other embodiments, the association of albumin with the imaging agent is non-covalent.

Modern molecular imaging has been established for the diagnosis of diseases and for tracing carrier molecules and determining their uptake and distribution in pathological sites. Molecular imaging provides methods such as magnetic resonance tomography (MRI), positron emission tomography (PET), computer tomography (CT), and single-photon emission computed tomography (SPECT).

Radiolabeling of carrier molecules such as peptides or proteins with gamma-emitting metal radionuclides such as ¹¹¹indium, ⁶⁷gallium or ^{99m}technetium is a suitable method for subsequent radioimaging of a patient using SPECT. DTPA (diethylenetriaminepentaacetic acid) is the chelating agent of choice for complexation forming stable ¹¹¹indium complexes, ⁶⁷gallium complexes and ^{99m}technetium complexes with fast labeling kinetics.

For the purpose of this invention new radioactive ¹¹¹indium complexes and ⁶⁷gallium complexes were designed that comprise a bifunctional chelating agent consisting of a DTPA derivative as the chelating agent connected to a linker incorporating a thiol-binding group (TBG) and a metal radionuclide selected from ¹¹¹indium or ⁶⁷gallium.

A thiol-binding group (TBG) is selected from a maleimide group, haloacetamide group, haloacetate group, pyridyldithio group, disulfide group, vinylcarbonyl group, aziridine group, and/or acetylene group possesses and it binds selectively and/or covalently reacts to thiol (—SH) groups of cysteines on the surface of proteins in a physiological environment. In some embodiments, the thiol-binding group is the maleimide group:

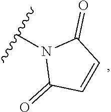 [SMILES: *N1C(=O)C=CC1=O]

that binds rapidly to cysteine-34 of endogenous or exogenous albumin, preferably to endogenous albumin in blood circulation after intravenous administration.

In some embodiments, a metal complex according to the present invention, abbreviated ¹¹¹In-C4-DTPA, has the following chemical structure as a conjugate acid or salt:

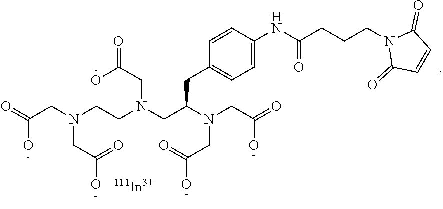

The binding of ¹¹¹In-C4-DTPA as well as the non-radioactive counterpart, In-C4-DTP A, to the cysteine-34 of endogenous and exogenous albumin and typical examples of SPECT/CT scans in tumor-bearing nude mice are illustrated in the figures.

The synthetic routes for preparing metal complexes according to the above embodiments are specified in the Examples and depicted in the 4 following synthetic schemes.

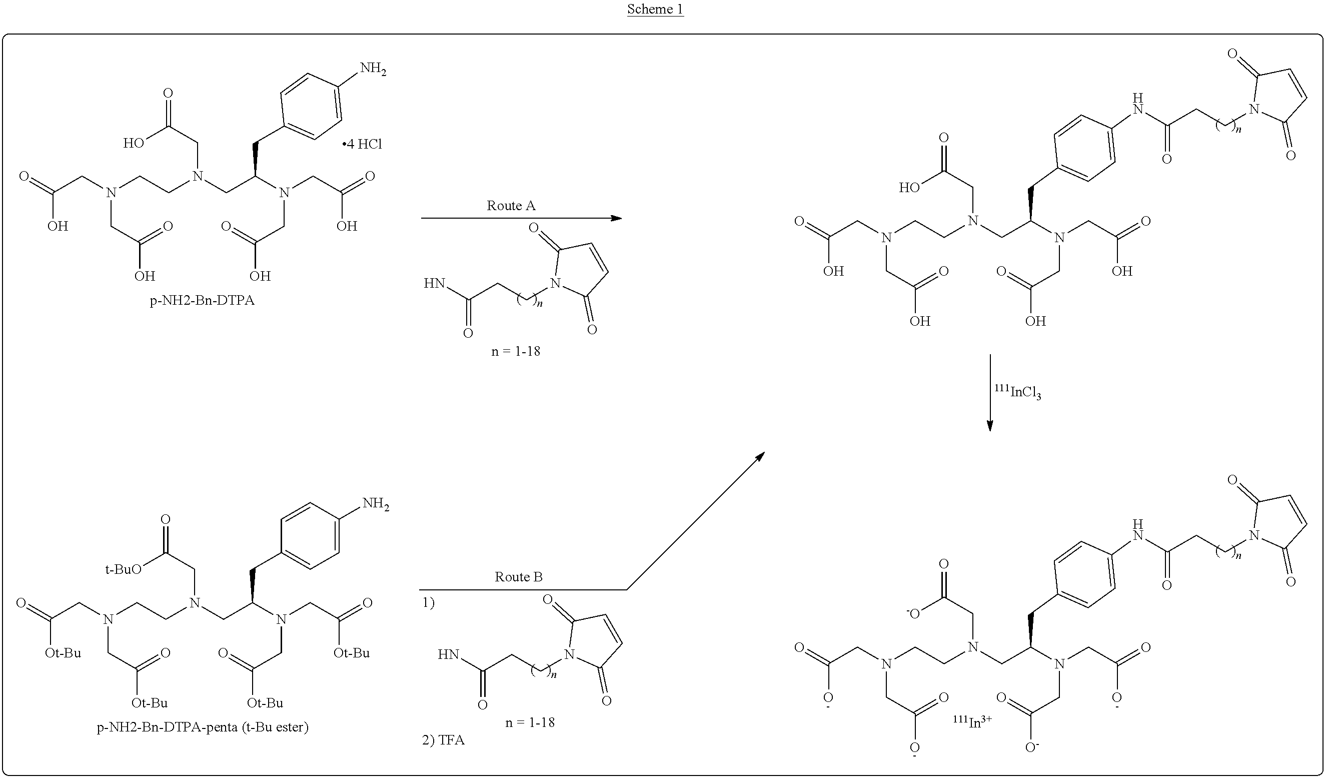

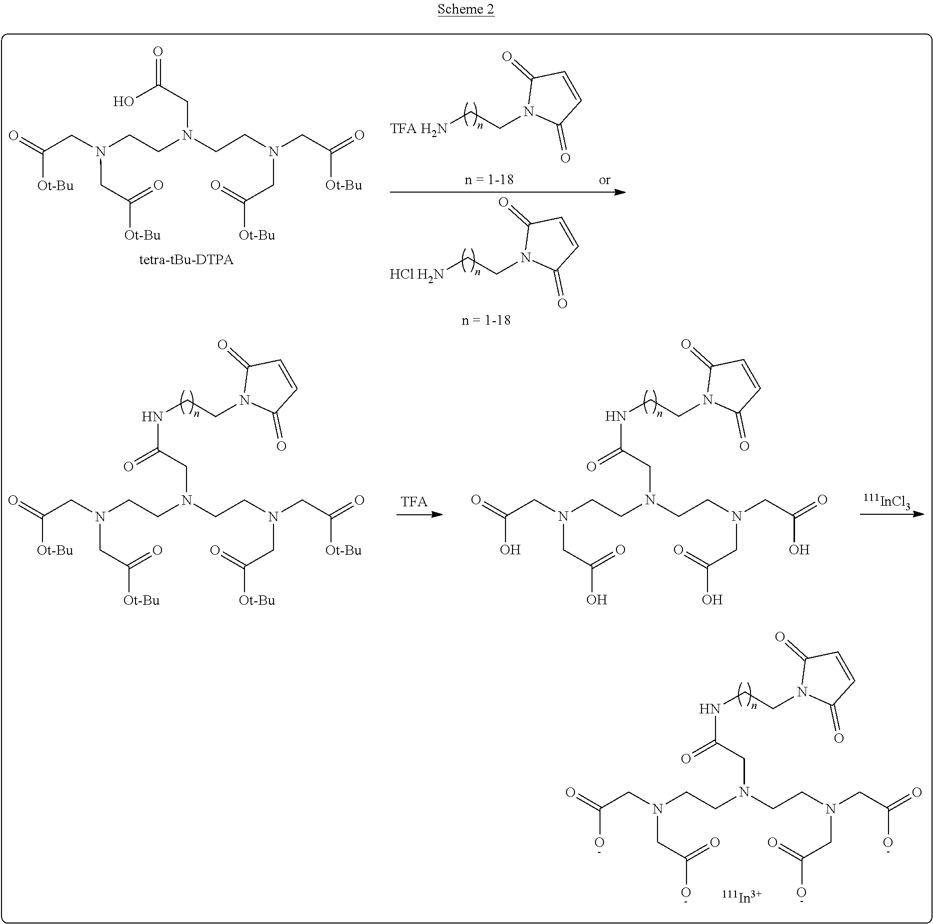

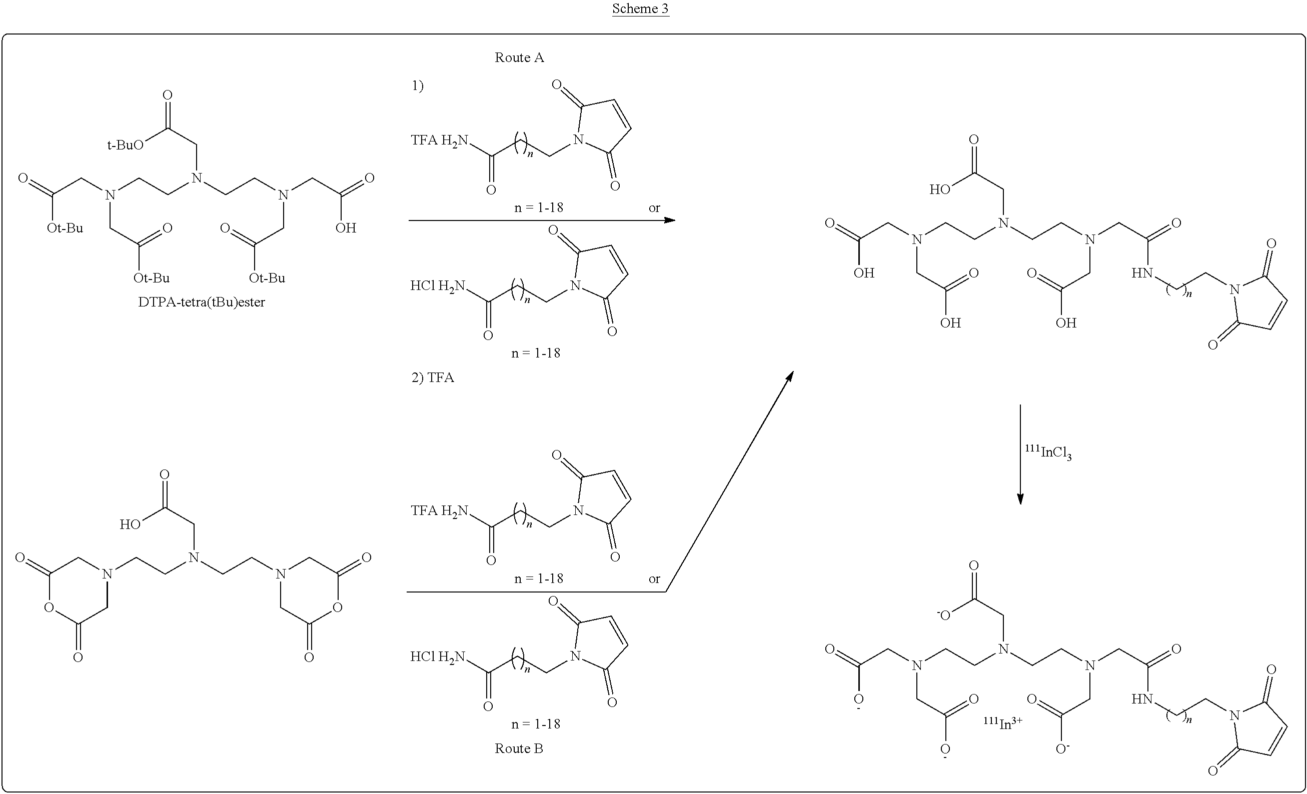

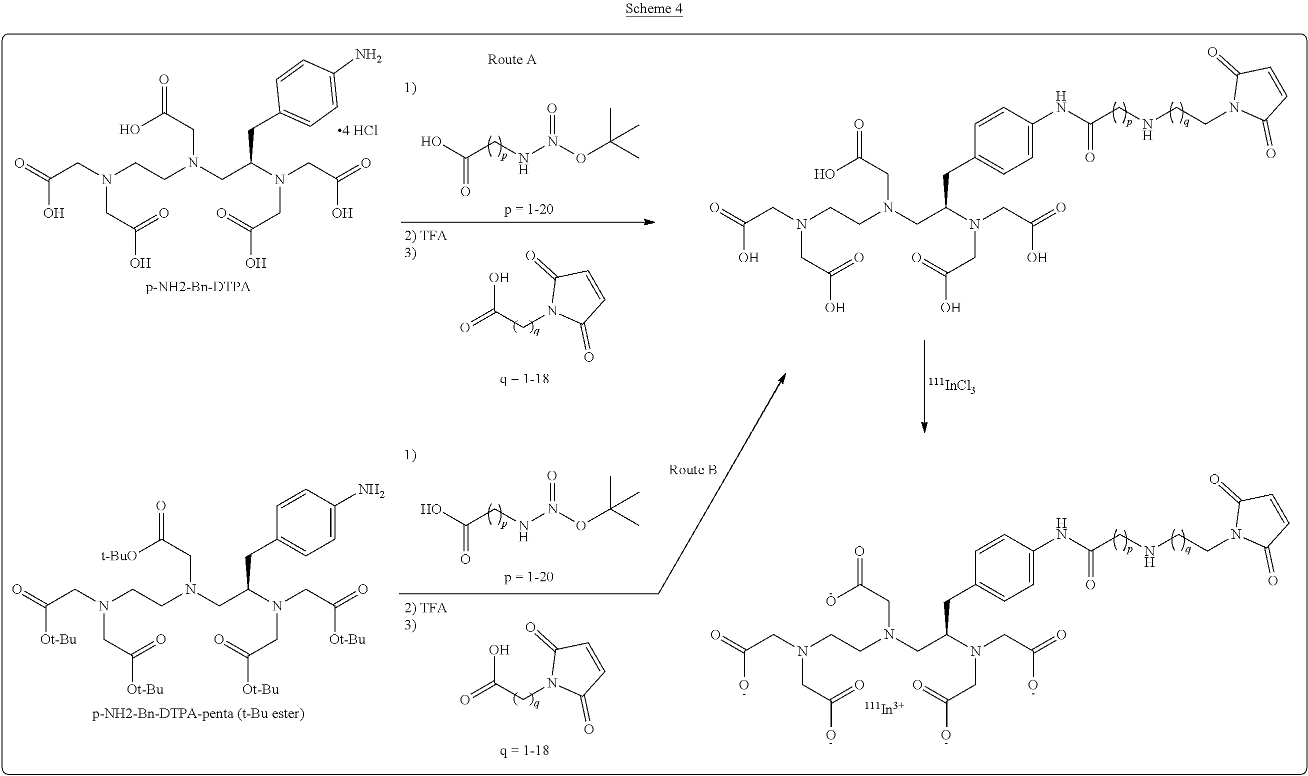

The synthetic procedure and characterization of certain bifunctional chelating agents and metal complexes according to the present invention is described in the Examples section.

### Pharmaceutical Compositions

Another aspect of the present disclosure provides a pharmaceutical composition comprising a metal complex of the disclosure together with a pharmaceutically acceptable carrier or excipient. Metal complexes or pharmaceutical compositions of the disclosure may be used in vitro or in vivo.

The total amount of a metal complex in a composition to be administered to a subject is one that is suitable for that subject. One of skill in the art would appreciate that different subjects may require different total amounts of the diagnostically effective substance. In some embodiments, the amount of the metal complex is a diagnostically effective amount. The skilled worker would be able to determine the amount of the metal complex in a composition needed for diagnostic imaging of a subject based on factors such as, for example, the age, weight, and physical condition of the subject. The concentration of the metal complex depends on its solubility in the intravenous administration solution and the volume of fluid that can be administered. For example, the concentration of the metal complex may be from about 0.001 mg/mL to about 8 mg/mL in the injectable composition.

The pharmaceutical compositions of the present invention may also contain diluents, fillers, salts, buffers, stabilizers, solubilizers, protectants and other materials well known in the art. The term “pharmaceutically acceptable” means a non-toxic material that does not interfere with the diagnostic effectiveness of the active ingredient(s). The characteristics of the carrier will depend on the route of administration.

In some embodiments, one or more excipients may be included in the composition. One of skill in the art would appreciate that the choice of any one excipient may influence the choice of any other excipient. For example, the choice of an excipient may preclude the use of one or more additional excipients because the combination of excipients would produce undesirable effects. One of skill in the art would be able to empirically determine which excipients, if any, to include in the compositions. Excipients may include, but are not limited to, co-solvents, solubilizing agents, buffers, pH adjusting agents, bulking agents, surfactants, encapsulating agents, tonicity-adjusting agents, stabilizing agents, protectants, and viscosity modifiers. In some embodiments, it may be beneficial to include a pharmaceutically acceptable carrier in the compositions.

In some embodiments, a solubilizing agent may be included in the pharmaceutical composition. Solubilizing agents may be useful for increasing the solubility of any of the components of the composition, including a compound or an excipient. The solubilizing agents described herein are not intended to constitute an exhaustive list, but are provided merely as exemplary solubilizing agents that may be used in the compositions. In certain embodiments, solubilizing agents include, but are not limited to, gentisic acid, myo-inositol, sodium citrate, citric acid, and combinations thereof, and any pharmaceutically acceptable salts and/or combinations thereof.

The pH of the compositions may be any pH that provides desirable properties for the formulation or composition. Desirable properties may include, for example, compound or metal complex stability, increased metal complex retention as compared to compositions at other pH values, and improved filtration efficiency. In some embodiments, the pH value of the compositions may be from about 3.0 to about 8.0, e.g., from about 3.0 to about 5.0. In particular embodiments, the pH value of the compositions may be 3.0±0.1, 3.5±0.1, 4.0±0.1, 4.5±0.1, 5.0±0.1.

In some embodiments, it may be beneficial to buffer the pH by including one or more buffers in the compositions. In certain embodiments, a buffer may have a pKa of, for example, about 3.0, about 3.5, about 4, about 4.5, about 5, about 5.5, about 6.0, or about 6.5. One of skill in the art would appreciate that an appropriate buffer may be chosen for inclusion in compositions based on its pKa and other properties. Buffers are well known in the art. Accordingly, the buffers described herein are not intended to constitute an exhaustive list, but are provided merely as exemplary buffers that may be used in the formulations or compositions of the invention. In certain embodiments, a buffer includes, but is not limited to Tris, Tris-HCl, potassium phosphate, sodium phosphate, sodium citrate, sodium ascorbate, combinations of sodium and potassium phosphate, Tris/Tris-HCl, sodium bicarbonate, arginine phosphate, arginine hydrochloride, histidine hydrochloride, cacodylate, succinate, 2-(N-morpholino)ethanesulfonic acid (MES), maleate, bis-tris, phosphate, carbonate, and any pharmaceutically acceptable salts and/or combinations thereof.

In some embodiments, a pH-adjusting agent may be included in the compositions. Modifying the pH of a composition may have beneficial effects on, for example, the stability or solubility of a metal complex, or may be useful in making a composition suitable for parenteral administration. pH-adjusting agents are well known in the art. Accordingly, the pH-adjusting agents described herein are not intended to constitute an exhaustive list, but are provided merely as exemplary pH-adjusting agents that may be used in the compositions. pH-adjusting agents may include, for example, acids and bases. In some embodiments, a pH-adjusting agent includes, but is not limited to, acetic acid, hydrochloric acid, phosphoric acid, sodium hydroxide, sodium carbonate, and combinations thereof.

In some embodiments, a bulking agent may be included in the compositions. Bulking agents are commonly used in lyophilized compositions to provide added volume to the composition and to aid visualization of the composition, especially in instances where the lyophilized pellet would otherwise be difficult to see. Bulking agents also may help prevent a blowout of the active component(s) of a pharmaceutical composition and/or to aid cryoprotection of the composition. Bulking agents are well known in the art. Accordingly, the bulking agents described herein are not intended to constitute an exhaustive list, but are provided merely as exemplary bulking agents that may be used in the compositions.

Exemplary bulking agents may include carbohydrates, monosaccharides, disaccharides, polysaccharides, sugar alcohols, amino acids, and sugar acids, and combinations thereof. Carbohydrate bulking agents include, but are not limited to, mono-, di-, or poly-carbohydrates, starches, aldoses, ketoses, amino sugars, glyceraldehyde, arabinose, lyxose, pentose, ribose, xylose, galactose, glucose, hexose, idose, mannose, talose, heptose, glucose, fructose, methyl a-D-glucopyranoside, maltose, lactone, sorbose, erythrose, threose, arabinose, allose, altrose, gulose, idose, talose, erythrulose, ribulose, xylulose, psicose, tagatose, glucosamine, galactosamine, arabinans, fructans, fucans, galactans, galacturonans, glucans, mannans, xylans, inulin, levan, fucoidan, carrageenan, galactocarolose, pectins, amylose, pullulan, glycogen, amylopectin, cellulose, pustulan, chitin, agarose, keratin, chondroitin, dermatan, hyaluronic acid, xanthin gum, sucrose, trehalose, dextran, and lactose. Sugar alcohol bulking agents include, but are not limited to, alditols, inositols, sorbitol, and mannitol. Sugar acid bulking agents include, but are not limited to, aldonic acids, uronic acids, aldaric acids, gluconic acid, isoascorbic acid, ascorbic acid, glucaric acid, glucuronic acid, gluconic acid, glucaric acid, galacturonic acid, mannuronic acid, neuraminic acid, pectic acids, and alginic acid. Amino acid bulking agents include, but are not limited to, glycine, histidine, and proline.

In some embodiments, a surfactant may be included in the compositions. Surfactants, in general, reduce the surface tension of a liquid composition. This may provide beneficial properties such as improved ease of filtration. Surfactants also may act as emulsifying agents and/or solubilizing agents. Surfactants are well known in the art. Accordingly, the surfactants described herein are not intended to constitute an exhaustive list, but are provided merely as exemplary surfactants that may be used in the formulations or compositions of the invention. Surfactants that may be included include, but are not limited to, sorbitan esters such as polysorbates (e.g., polysorbate 20 and polysorbate 80), lipopolysaccharides, polyethylene glycols (e.g., PEG 400 and PEG 3000), poloxamers (i.e., pluronics), ethylene oxides and polyethylene oxides (e.g., Triton X-100), saponins, phospholipids (e.g., lecithin), and combinations thereof.

In some embodiments, a tonicity-adjusting agent may be included in the compositions. The tonicity of a liquid composition is an important consideration when administering the composition to a subject, for example, by parenteral administration. Tonicity-adjusting agents, thus, may be used to help make a composition suitable for administration. Tonicity-adjusting agents are well known in the art. Accordingly, the tonicity-adjusting agents described herein are not intended to constitute an exhaustive list, but are provided merely as exemplary tonicity-adjusting agents that may be used in the compositions. Tonicity-adjusting agents may be ionic or non-ionic and include, but are not limited to, inorganic salts, amino acids, carbohydrates, sugars, sugar alcohols, and carbohydrates. Exemplary inorganic salts may include sodium chloride, potassium chloride, sodium sulfate, and potassium sulfate. An exemplary amino acid is glycine. Exemplary sugars may include sugar alcohols such as glycerol, propylene glycol, glucose, sucrose, lactose, and mannitol.

In some embodiments, a stabilizing agent may be included in the compositions. Stabilizing agents help increase the stability of a metal complex in the compositions. This may occur by, for example, reducing degradation or preventing aggregation of a metal complex. Without wishing to be bound by theory, mechanisms for enhancing stability may include sequestration of the metal complex from a solvent or inhibiting free radical oxidation of the therapeutically effective substance. Stabilizing agents are well known in the art. Accordingly, the stabilizing agents described herein are not intended to constitute an exhaustive list, but are provided merely as exemplary stabilizing agents that may be used in the compositions. Stabilizing agents may include, but are not limited to, emulsifiers and surfactants.

In some embodiments, a protectant may be included in the compositions. Protectants are agents that protect a diagnostically active ingredient (e.g., a diagnostically effective substance or compound, e.g. imaging agent) from an undesirable condition (e.g., instability caused by freezing or lyophilization, or oxidation). Protectants can include, for example, cryoprotectants, lyoprotectants, and antioxidants. For example, a cryoprotectant could be included in a reconstituted lyophilized formulation so that the formulation could be frozen before dilution for intravenous administration. Cryoprotectants are well known in the art. Accordingly, the cryoprotectants described herein are not intended to constitute an exhaustive list, but are provided merely as exemplary cryoprotectants that may be used in the compositions. Cryoprotectants include, but are not limited to, solvents, surfactants, encapsulating agents, stabilizing agents, viscosity modifiers, and combinations thereof. Cryoprotectants may include, for example, disaccharides (e.g., sucrose, lactose, maltose, and trehalose), polyols (e.g., glycerol, mannitol, sorbitol, and dulcitol), glycols (e.g., ethylene glycol, polyethylene glycol and propylene glycol).

Lyoprotectants are useful in stabilizing the components of a composition. For example, a diagnostically effective substance could be lyophilized with a lyoprotectant prior to reconstitution. Lyoprotectants are well known in the art. Accordingly, the lyoprotectants described herein are not intended to constitute an exhaustive list, but are provided merely as exemplary lyoprotectants that may be used in the compositions. Lyoprotectants include, but are not limited to, solvents, surfactants, encapsulating agents, stabilizing agents, viscosity modifiers, and combinations thereof. Exemplary lyoprotectants may be, for example, sugars and polyols. Trehalose, sucrose, dextran, and hydroxypropyl-beta-cyclodextrin are non-limiting examples of lyoprotectants.

Antioxidants are useful in preventing oxidation of the components of a composition. Oxidation may result in aggregation of an imaging agent or other detrimental effects to the purity of the imaging agent. Antioxidants are well known in the art. Accordingly, the antioxidants described herein are not intended to constitute an exhaustive list, but are provided merely as exemplary antioxidants that may be used in the compositions. Antioxidants may be, for example, gentisic acid, sodium ascorbate, citrate, thiols, metabisulfite, and combinations thereof.

In some embodiments, a viscosity modifying agent may be included in the composition. Viscosity modifiers change the viscosity of liquid compositions. This may be beneficial because viscosity plays an important role in the ease with which a liquid composition is filtered. A composition may be filtered prior to lyophilization and reconstitution, or after reconstitution. Viscosity modifiers are well known in the art. Accordingly, the viscosity modifiers described herein are not intended to constitute an exhaustive list, but are provided merely as exemplary viscosity modifiers that may be used in the compositions. Viscosity modifiers include solvents, solubilizing agents, surfactants, and encapsulating agents. Exemplary viscosity modifiers that may be included in compositions include, but are not limited to, N-acetyl-DL-tryptophan and N-acetyl-cysteine.

The compositions may be administered in a variety of conventional ways. Exemplary routes of administration that can be used include oral, parenteral, intravenous, intra-arterial, cutaneous, subcutaneous, intramuscular, topical, intracranial, intraorbital, ophthalmic, intravitreal, intraventricular, intracapsular, intraspinal, intracisternal, intraperitoneal, intranasal, aerosol, central nervous system (CNS) administration, or administration by suppository. In some embodiments, the compositions are suitable for parenteral administration. These compositions may be administered, for example, intraperitoneally, intravenously, or intrathecally. In some embodiments, the compositions are injected intravenously. In some embodiments, a reconstituted formulation can be prepared by reconstituting a lyophilized metal complex composition in a reconstitution liquid comprising e.g. an alcohol, DMSO, and/or polyethylene glycol and water and/or a salt buffer. Such reconstitution may comprise adding the reconstitution liquid and mixing, for example, by swirling or vortexing the mixture. The reconstituted formulation then can be made suitable for injection by mixing e.g., Lactated Ringer's solution, 5% glucose solution, isotonic saline or a suitable salt buffer with the formulation to create an injectable composition. One of skill in the art would appreciate that a method of administering a diagnostically effective substance formulation or composition would depend on factors such as the age, weight, and physical condition of the subject being treated, and the disease or condition being treated. The skilled worker would, thus, be able to select a method of administration optimal for a subject on a case-by-case basis.

In some embodiments, the invention provides metal complexes and compositions for use in the radioimaging of a cancer, a viral disease, autoimmune disease, acute or chronic inflammatory disease, and/or a disease caused by bacteria, fungi, or other micro-organisms.

In some embodiments, the compounds, compositions, or metal complex disclosed herein may be used in the manufacture of a medicament for the radioimaging of a disease selected from a cancer, a virus disease, autoimmune disease, acute or chronic inflammatory disease, and a disease caused by bacteria, fungi, or other micro-organisms.

In some embodiments, the cancer is a blood cancer or a solid tumor cancer. In some embodiments, the cancer is selected from carcinoma, sarcoma, leukemia, lymphoma, multiple myeloma, or melanoma.

In some embodiments, the cancer is adenocarcinoma, uveal melanoma, acute leukemia, acoustic neuroma, ampullary carcinoma, anal carcinoma, astrocytoma's, basalioma, pancreatic cancer, connective tissue tumor, bladder cancer, bronchial carcinoma, non-small cell bronchial carcinoma, breast cancer, Burkitt's lymphoma, corpus carcinoma, CUP syndrome, colon cancer, cancer of the small intestine, ovarian cancer, endometrial carcinoma, gallbladder cancer, gallbladder carcinomas, uterine cancer, cervical cancer, neck, nose and ear tumors, hematological neoplasia's, hairy cell leukemia, urethral cancer, skin cancer, gliomas, testicular cancer, Kaposi's sarcoma, laryngeal cancer, bone cancer, colorectal carcinoma, head/neck tumors, colon carcinoma, craniopharyngeoma, liver cancer, leukemia, lung cancer, non-small cell lung cancer, Hodgkin's lymphoma, non-Hodgkin's lymphoma, stomach cancer, colon cancer, medulloblastoma, melanoma, meningioma, kidney cancer, renal cell carcinomas, oligodendroglioma, esophageal carcinoma, osteolytic carcinomas and osteoplastic carcinomas, osteosarcoma, ovarian carcinoma, pancreatic carcinoma, penile cancer, prostate cancer, tongue cancer, ovary carcinoma or lymph gland cancer.

### Methods of Diagnosing/Imaging and Treatment

The metal complexes and compositions described herein are useful for a variety of clinical applications. Thus, another aspect of the disclosure provides methods of diagnosing, imaging and/or treating a subject.

In some embodiments, the metal complexes and compositions of this invention can be administered intravenously and covalently bind selectively and rapidly to endogenous albumin in the blood circulation for the purpose of radioimaging (e.g., with Single Photon Emission Computed Tomography (SPECT)).

In some embodiments, the invention provides a method for the imaging of a malignant disease comprising administering to a subject in need thereof a diagnostically effective amount of a metal complex or a pharmaceutical composition comprising a metal complex described herein.

The disclosure also provides a method of imaging a condition or disease in a subject, said condition or disease selected from a cancer, a viral disease, autoimmune disease, acute or chronic inflammatory disease, and a disease caused by bacteria, fungi, or other micro-organisms, comprising administering to the subject a metal complex or a pharmaceutical composition as described herein.

In some embodiments, the invention provides a method for diagnosing a disease selected from a cancer, a viral disease, autoimmune disease, acute or chronic inflammatory disease, and a disease caused by bacteria, fungi, or other micro-organisms, comprising administering to a subject in need thereof an effective amount of a metal complex or a pharmaceutical composition comprising a metal complex described herein and subsequent imaging. In some embodiments, the disease is cancer.

In some embodiments, the invention provides a method of diagnosing cancer in a subject, the method comprising: administering a detectable amount of a metal complex or a pharmaceutical composition comprising a metal complex as disclosed herein (including, e.g., a metal complex comprising a radiolabel) to the subject; imaging the subject after administering the metal complex or pharmaceutical composition comprising a metal complex to detect a signal from a radiolabel of the metal complex, wherein a presence of the radiolabel in a tissue indicates that the tissue is cancerous, thereby diagnosing cancer in the subject. In some embodiments, the invention provides a method of diagnosing cancer in a subject, the method comprising: administering a detectable amount of a metal complex or a pharmaceutical composition comprising a metal complex as disclosed herein (including, e.g., a metal complex comprising a radiolabel) to the subject; performing imaging on the subject after administering the metal complex or pharmaceutical composition comprising a metal complex to detect a signal from a radiolabel of the metal complex in a tissue in said subject, wherein a presence of the radiolabel in the tissue indicates that the tissue is cancerous, thereby diagnosing cancer in the subject. In some embodiments, the invention provides a method of diagnosing cancer in a subject, the method comprising: administering a detectable amount of a metal complex or a pharmaceutical composition comprising a metal complex as disclosed herein (including, e.g., a metal complex comprising a radiolabel) to the subject; performing imaging on the subject after administering the metal complex or pharmaceutical composition comprising a metal complex to detect a signal from a radiolabel of the metal complex in a tissue in said subject, wherein detecting a presence of the signal from the radiolabel in the tissue indicates that the tissue is cancerous, thereby diagnosing cancer in the subject.

In some embodiments, the invention provides a method of diagnosing cancer in a subject, the method comprising: administering a detectable amount of a metal complex or a pharmaceutical composition comprising a metal complex disclosed herein (including, e.g., a metal complex comprising a radiolabel) to the subject; imaging the subject after administering the radiolabeled metal complex or a pharmaceutical composition comprising the metal complex to detect a signal from a radiolabel of the metal complex, wherein a presence of a higher accumulation of the signal in a tissue in comparison to noncancerous tissue of the same type indicates that the tissue is cancerous, thereby diagnosing cancer in the subject. In some embodiments, the invention provides a method of diagnosing cancer in a subject, the method comprising: administering a detectable amount of a metal complex or a pharmaceutical composition comprising a metal complex disclosed herein (including, e.g., a metal complex comprising a radiolabel) to the subject; performing imaging on the subject after administering the radiolabeled metal complex or a pharmaceutical composition comprising the metal complex to detect a signal from a radiolabel of the metal complex, wherein detecting a presence of a higher amount of the signal from the radiolabel in a tissue of the subject in comparison to noncancerous tissue of the same type indicates that the tissue is cancerous, thereby diagnosing cancer in the subject.

In some embodiments, the invention provides a method of diagnosing cancer in a subject, the method comprising: administering to the subject a metal complex or a pharmaceutical composition comprising a metal complex disclosed herein (including, e.g., a metal complex comprising a radiolabel), wherein the metal complex binds to albumin to form a metal complex-albumin conjugate, wherein the metal complex-albumin conjugate accumulates in cancerous tissue; performing imaging on the subject after administering a metal complex or pharmaceutical composition comprising a metal complex disclosed herein to detect a signal from a radiolabel of the metal complex in a tissue in said subject, wherein detecting a presence of the signal from the radiolabel in the tissue indicates that the tissue is cancerous, thereby diagnosing cancer in the subject. In some embodiments, the invention provides a method of diagnosing cancer in a subject, the method comprising: administering to the subject a metal complex or a pharmaceutical composition comprising a metal complex disclosed herein (including, e.g., a metal complex comprising a radiolabel), wherein the metal complex binds to albumin to form a metal complex-albumin conjugate, wherein the metal complex-albumin conjugate accumulates in cancerous tissue; performing imaging on the subject after administering a metal complex or pharmaceutical composition comprising a metal complex disclosed herein to detect the metal complex (or the radiolabel of the metal complex) in a tissue in said subject, wherein detecting a presence of the metal complex (or the radiolabel of the metal complex) in the tissue indicates that the tissue is cancerous, thereby diagnosing cancer in the subject.

In some embodiments, the invention provides a method of treating cancer in a subject, the method comprising: administering to the subject a metal complex or a pharmaceutical composition comprising a metal complex disclosed herein (including, e.g., a metal complex comprising a radiolabel), wherein the metal complex binds to albumin to form a metal complex-albumin conjugate, wherein the metal complex-albumin conjugate accumulates in cancerous tissue; performing imaging on the subject after administering the metal complex or pharmaceutical composition comprising a metal complex to detect a signal from a radiolabel of the metal complex in a tissue in said subject, wherein detecting a presence of the signal from the radiolabel in the tissue indicates that the tissue is cancerous; diagnosing the subject with cancer in the tissue; and administering a therapeutically effective amount of a chemotherapeutic agent to the subject. In some embodiments, the invention provides a method of treating cancer in a subject, the method comprising: administering to the subject a metal complex or a pharmaceutical composition comprising a metal complex disclosed herein (including, e.g., a metal complex comprising a radiolabel), wherein the metal complex binds to albumin to form a metal complex-albumin conjugate, wherein the metal complex-albumin conjugate accumulates in cancerous tissue; performing imaging on the subject after administering the metal complex or pharmaceutical composition comprising a metal complex to detect the metal complex (or the radiolabel of the metal complex) in a tissue in said subject, wherein detecting a presence of the radiolabel in the tissue indicates that the tissue is cancerous; diagnosing the subject with cancer in the tissue; and administering a therapeutically effective amount of a chemotherapeutic agent to the subject.

In some embodiments, the invention provides a method of diagnosing and treating a subject with a cancer responsive to an albumin-binding chemotherapeutic agent, the method comprising: administering to the subject a metal complex or a pharmaceutical composition comprising a metal complex disclosed herein (including, e.g., a metal complex comprising a radiolabel), wherein the metal complex binds to albumin to form a metal complex-albumin conjugate, wherein the metal complex-albumin conjugate accumulates in cancerous tissue; performing imaging on the subject after administering the metal complex or pharmaceutical composition comprising a metal complex to detect a signal from a radiolabel of the metal complex in a tissue in said subject, wherein detecting a presence of the signal from the radiolabel in the tissue indicates that the tissue is cancerous; diagnosing the subject with cancer that is responsive to treatment with the albumin-binding chemotherapeutic agent; and administering a therapeutically effective amount of the albumin-binding chemotherapeutic agent to the subject. In some embodiments, the invention provides a method of diagnosing and treating a subject with a cancer responsive to an albumin-binding chemotherapeutic agent, the method comprising: administering to the subject a metal complex or a pharmaceutical composition comprising a metal complex disclosed herein (including, e.g., a metal complex comprising a radiolabel), wherein the metal complex binds to albumin to form a metal complex-albumin conjugate, wherein the metal complex-albumin conjugate accumulates in cancerous tissue; performing imaging on the subject after administering the metal complex or pharmaceutical composition comprising a metal complex to detect the presence of the metal complex (or the radiolabel from the metal complex) in a tissue in said subject, wherein detecting a presence of the metal complex (or the radiolabel from the metal complex) in the tissue indicates that the tissue is cancerous; diagnosing the subject with cancer that is responsive to treatment with the albumin-binding chemotherapeutic agent; and administering a therapeutically effective amount of the albumin-binding chemotherapeutic agent to the subject.

In some embodiments, the invention provides a method comprising: administering to a subject a metal complex or a pharmaceutical composition comprising a metal complex disclosed herein (including, e.g., a metal complex comprising a radiolabel), wherein the metal complex binds to albumin to form a metal complex-albumin conjugate, wherein the metal complex-albumin conjugate accumulates in cancerous tissue; performing imaging on the subject after administering the metal complex or pharmaceutical composition comprising a metal complex to detect a signal from a radiolabel of the metal complex in a tissue in said subject, wherein detecting a presence of the signal from the radiolabel in the tissue indicates that the tissue is cancerous; diagnosing the subject with cancer in the tissue; and administering a therapeutically effective amount of an albumin-binding chemotherapeutic agent to the subject. In some embodiments, the invention provides a method comprising: administering to a subject a metal complex or a pharmaceutical composition comprising a metal complex disclosed herein (including, e.g., a metal complex comprising a radiolabel), wherein the metal complex binds to albumin to form a metal complex-albumin conjugate, wherein the metal complex-albumin conjugate accumulates in cancerous tissue; performing imaging on the subject after administering the metal complex or pharmaceutical composition comprising a metal complex to detect the metal complex (or the radiolabel from the metal complex) in a tissue in said subject, wherein detecting a presence of the metal complex (or the radiolabel from the metal complex) in the tissue indicates that the tissue is cancerous; diagnosing the subject with cancer in the tissue; and administering a therapeutically effective amount of an albumin-binding chemotherapeutic agent to the subject.

In some embodiments, the invention provides a method comprising: administering to a subject a metal complex or a pharmaceutical composition comprising a metal complex disclosed herein (including, e.g., a metal complex comprising a radiolabel), wherein the metal complex binds to albumin to form a metal complex-albumin conjugate, wherein the metal complex-albumin conjugate accumulates in cancerous tissue; performing imaging on the subject after administering the metal complex or pharmaceutical composition comprising a metal complex to detect a signal from a radiolabel of the metal complex, wherein detection of a presence of the signal from the radiolabel in the tissue indicates that the tissue is cancerous; diagnosing the subject with cancer in the tissue; classifying the subject as being responsive to an albumin-binding chemotherapeutic agent; and administering a therapeutically effective amount of the albumin-binding chemotherapeutic agent to the subject. In some embodiments, the invention provides a method comprising: administering to a subject a metal complex or a pharmaceutical composition comprising a metal complex disclosed herein (including, e.g., a metal complex comprising a radiolabel), wherein the metal complex binds to albumin to form a metal complex-albumin conjugate, wherein the metal complex-albumin conjugate accumulates in cancerous tissue; performing imaging on the subject after administering the metal complex or pharmaceutical composition comprising a metal complex to detect the metal complex (or the radiolabel from the metal complex), wherein detection of the metal complex (or the radiolabel from the metal complex) in the tissue indicates that the tissue is cancerous; diagnosing the subject with cancer in the tissue; classifying the subject as being responsive to an albumin-binding chemotherapeutic agent; and administering a therapeutically effective amount of the albumin-binding chemotherapeutic agent to the subject.

In some embodiments, the invention provides a method of imaging a target site in a subject comprising: administering to the subject a metal complex or a pharmaceutical composition comprising a metal complex disclosed herein (including, e.g., a metal complex comprising a radiolabel), wherein the metal complex binds to albumin to form a metal complex-albumin conjugate, wherein the metal complex-albumin conjugate accumulates at the target site; and performing imaging on the target site in the subject after administering the metal complex or pharmaceutical composition comprising a metal complex.

In some embodiments, the invention provides a method for assessing the responsiveness of a subject having a cancer to an albumin-binding chemotherapeutic agent comprising: administering to a subject a metal complex or a pharmaceutical composition comprising a metal complex disclosed herein (including, e.g., a metal complex comprising a radiolabel), wherein the metal complex binds to albumin to form a metal complex-albumin conjugate, wherein the metal complex-albumin conjugate accumulates in cancerous tissue; performing imaging on the subject after administering the metal complex or pharmaceutical composition comprising a metal complex to detect a signal from a radiolabel of the metal complex, wherein detection of a presence of the signal from the radiolabel in the tissue indicates that the tissue is cancerous; diagnosing the subject with cancer in the tissue; and classifying the subject as having a cancer responsive to the albumin-binding chemotherapeutic agent. In some embodiments, the invention provides a method for assessing the responsiveness of a subject having a cancer to an albumin-binding chemotherapeutic agent comprising: administering to a subject a metal complex or a pharmaceutical composition comprising a metal complex disclosed herein (including, e.g., a metal complex comprising a radiolabel), wherein the metal complex binds to albumin to form a metal complex-albumin conjugate, wherein the metal complex-albumin conjugate accumulates in cancerous tissue; performing imaging on the subject after administering the metal complex or pharmaceutical composition comprising a metal complex to detect the metal complex (or the radiolabel from the metal complex), wherein detection of a presence of the metal complex (or the radiolabel from the metal complex) in the tissue indicates that the tissue is cancerous; diagnosing the subject with cancer in the tissue; and classifying the subject as having a cancer responsive to the albumin-binding chemotherapeutic agent.

In some embodiments, the invention provides a method for selectively accumulating a radiolabel of a metal complex disclosed herein, inside target cells of a subject, comprising administering the metal complex or a pharmaceutical composition comprising a metal complex described herein to the subject. In some embodiments the target cells are cancer cells.

In some embodiments, the invention provides a method for delivering a radiolabel to a target site of a subject, the method comprising: administering to the subject a metal complex or a pharmaceutical composition comprising a metal complex disclosed herein, wherein the metal complex comprises a radiolabel and wherein the metal complex binds to albumin to form a metal complex-albumin conjugate, wherein the metal complex-albumin conjugate accumulates at the target site.

In some embodiments, the imaging is performed at a time point between about 5 minutes and 96 hours after administration of the metal complex or pharmaceutical composition. In some embodiments, the imaging is performed at a time point between about 5 minutes and 10 hours after administration of the metal complex or pharmaceutical composition. In some embodiments, the imaging is performed at a time point between about 5 minutes and 5 hours after administration of the metal complex or pharmaceutical composition. In some embodiments, the imaging is performed at a time point between about 5 minutes and 4 hours after administration of the metal complex or pharmaceutical composition. In some embodiments, the imaging is performed at a time point between about 5 minutes and 3 hours after administration of the metal complex or pharmaceutical composition. In some embodiments, the imaging is performed at a time point between about 5 minutes and 2 hours after administration of the metal complex or pharmaceutical composition. In some embodiments, the imaging is performed at a time point between about 5 minutes and 1 hour after administration of the metal complex or pharmaceutical composition. In some embodiments, the imaging is performed at a time point between about 5 minutes and 30 minutes after administration of the metal complex or pharmaceutical composition. In some embodiments, the imaging is performed at a time point between about 10 and 20 hours after administration of the metal complex or pharmaceutical composition. In some embodiments, the imaging is performed at a time point between about 20 and 30 hours after administration of the metal complex or pharmaceutical composition. In some embodiments, the imaging is performed at a time point between about 30 and 40 hours after administration of the metal complex or pharmaceutical composition. In some embodiments, the imaging is performed at a time point between about 40 and 50 hours after administration of the metal complex or pharmaceutical composition. In some embodiments, the imaging is performed at a time point between about 50 and 60 hours after administration of the metal complex or pharmaceutical composition. In some embodiments, the imaging is performed at a time point between about 70 and 80 hours after administration of the metal complex or pharmaceutical composition. In some embodiments, the imaging is performed at a time point between about 80 and 90 hours after administration of the metal complex or pharmaceutical composition. In some embodiments, the imaging is performed at a time point greater than 90 hours after administration of the metal complex or pharmaceutical composition. In some embodiments the imaging is performed at a time point about 5, 10, 15, 20, 25, 30 35, 40, 45, 50 or 55 minutes after administration of the metal complex or pharmaceutical composition. In some embodiments, the imaging is performed at a time point about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95 or 96 hours after administration of the metal complex or pharmaceutical composition.

In some embodiments, the metal complex or pharmaceutical composition comprising a metal complex is administered to the subject as a metal complex-albumin conjugate formed ex vivo. In some embodiments, the metal complex is administered to the subject and the metal complex-albumin conjugate is formed in vivo.

In some embodiments, the metal complex or pharmaceutical composition comprising a metal complex is administered the subject as a metal complex-albumin conjugate formed ex vivo, wherein metal complex-albumin conjugate is formed by: conjugation of albumin to a moiety corresponding to the TBG of the metal complex; followed by chelation of M. Albumin may be autologous or heterologous to the subject.

In some embodiments, the metal complex or pharmaceutical composition comprising a metal complex is administered to the subject as a metal complex-albumin conjugate formed ex vivo, wherein metal complex-albumin conjugate is formed by: chelation of M to form the metal complex; followed by conjugation of albumin to the TBG of the metal complex to form the metal complex-albumin conjugate. Albumin may be autologous or heterologous to the subject. In some embodiments, the metal complex or pharmaceutical composition comprising a metal complex is administered to the subject and the metal complex-albumin conjugate is formed in vivo.

In some embodiments, the diagnostic methods described herein are for the radioimaging of a disease selected from a cancer, a virus disease, autoimmune disease, acute or chronic inflammatory disease, and a disease caused by bacteria, fungi, or other micro-organisms.

In some embodiments, the cancer is a blood cancer or a solid tumor cancer. In some embodiments, the cancer is selected from carcinoma, sarcoma, leukemia, lymphoma, multiple myeloma, or melanoma. In some embodiments, the cancer is adenocarcinoma, uveal melanoma, acute leukemia, acoustic neuroma, ampullary carcinoma, anal carcinoma, astrocytoma's, basalioma, pancreatic cancer, connective tissue tumor, bladder cancer, bronchial carcinoma, non-small cell bronchial carcinoma, breast cancer, Burkitt's lymphoma, corpus carcinoma, CUP syndrome, colon cancer, cancer of the small intestine, ovarian cancer, endometrial carcinoma, gallbladder cancer, gallbladder carcinomas, uterine cancer, cervical cancer, neck, nose and ear tumors, hematological neoplasia's, hairy cell leukemia, urethral cancer, skin cancer, gliomas, testicular cancer, Kaposi's sarcoma, laryngeal cancer, bone cancer, colorectal carcinoma, head/neck tumors, colon carcinoma, craniopharyngeoma, liver cancer, leukemia, lung cancer, non-small cell lung cancer, Hodgkin's lymphoma, non-Hodgkin's lymphoma, stomach cancer, colon cancer, medulloblastoma, melanoma, meningioma, kidney cancer, renal cell carcinomas, oligodendroglioma, esophageal carcinoma, osteolytic carcinomas and osteoplastic carcinomas, osteosarcoma, ovarian carcinoma, pancreatic carcinoma, penile cancer, prostate cancer, tongue cancer, ovary carcinoma or lymph gland cancer.

In some embodiments, the imaging is accomplished via positron emission tomography (PET) or single photon emission tomography (SPECT). In some embodiments, the imaging is accomplished additionally via magnetic resonance imaging (MRI) or computed tomography (CT).

In some embodiments, the subject is a mammal. In some embodiments, the subject is a bovine, a feline, a canine, a murine, an equine, or a human. In some embodiments, the subject is a human.

### Kits

In another aspect, the present disclosure provides a kit. In some embodiments, the kit comprises a metal complex as described herein. In some embodiments, the kit is for diagnosing cancer. In other embodiments, the kit is for diagnosing whether a subject suffering from cancer will be responsive to an albumin-binding chemotherapeutic agent. The kit is an assemblage of materials or components, including at least a metal complex as described herein or pharmaceutical composition comprising the metal complex.

In some embodiments, the kit is for an imaging technique to detect albumin in a subject. In other embodiments, the kit is for determining the extent of albumin uptake and distribution in pathological sites in a subject (e.g., in a tumor).

In some embodiments, the kit is for the diagnosis of cancer in a subject, comprising a metal complex or composition described herein; instructions to use the metal complex or composition for the diagnosis of cancer comprising instructions to administer the metal complex or composition to the subject; instructions to image the subject after administering the metal complex or composition to detect a signal from the label or from the radioactive isotope, wherein an abnormal accumulation of the signal indicates the presence of cancer in the subject.

In some embodiments, the kit is for diagnosing whether a subject suffering from cancer will be responsive to an albumin-binding chemotherapeutic agent, wherein the kit comprises a metal complex or composition described herein; instructions to use the metal complex or composition for diagnosing whether a subject suffering from cancer will be responsive to an albumin-binding chemotherapeutic agent comprising instructions to administer the metal complex or composition to the subject; instructions to image the subject after administering the metal complex or composition to detect a signal from the label or from the radioactive isotope, wherein an abnormal accumulation of the signal indicates the presence of cancer in the subject.

The exact nature of the components configured in the kit depends on its intended purpose. For example, some embodiments are configured for the purpose of diagnosing cancer. Other embodiments are configured to diagnose whether a subject suffering from cancer will be responsive to an albumin-binding chemotherapeutic agent. In some embodiments, the kit is configured particularly for the purpose of diagnosing mammalian subjects. In another embodiment, the kit is configured particularly for the purpose of diagnosing human subjects. In further embodiments, the kit is configured for veterinary applications, diagnosing subjects such as, but not limited to, farm animals, domestic animals, and laboratory animals.

Instructions for use may be included in the kit. “Instructions for use” typically include a tangible expression describing the technique to be employed in using the components of the kit to attain a desired outcome, such as to diagnose cancer in a subject or to diagnose whether a subject suffering from cancer will be responsive to an albumin-binding chemotherapeutic agent. Instructions may comprise, for example, instructions to administer the metal complex or composition to the subject; instructions to image the subject after administering the metal complex or composition to detect a signal from the label or from a radioactive isotope, wherein an accumulation of the signal indicates the presence of cancer in the subject or wherein an accumulation of the signal indicates that the subject will be response to an albumin-binding chemotherapeutic agent.

Optionally, the kit also contains other useful components, such as, diluents, buffers, pharmaceutically acceptable carriers, syringes, catheters, applicators, pipetting or measuring tools, or other useful components as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit. The packaging material is constructed by well-known methods, preferably to provide a sterile, contaminant-free environment. The packaging materials employed in the kit are those customarily utilized in diagnosing cancer and/or containing radioactive compositions. As used herein, the term “package” refers to a suitable solid matrix or material such as glass, plastic, paper, foil, and the like, capable of holding the individual kit components. The packaging material generally has an external label which indicates the contents and/or purpose of the kit and/or its components.

### Variations and Modifications

Variations, modifications, and other implementations of what is described herein will occur to those of ordinary skill without departing from the spirit and the scope of the invention. Accordingly, the invention is not to be limited to the preceding description or the following examples.

### Exemplification

With aspects of the invention now being generally described, these will be more readily understood by reference to the following examples, which are included merely for purposes of illustration of certain features and embodiments of the invention and are not intended to be limiting.

### Equivalents

Those skilled in the art will recognize, or be able to ascertain using no more than routine experimentation, numerous equivalents to the metal complexes, compositions, and methods of use thereof described herein. Such equivalents are considered to be within the scope of the invention.

### EXAMPLES

### Example 1

### Materials and Methods for Preparation of Metal Complexes

All reactions were carried out under N₂ inert atmosphere, unless otherwise stated. Commercially available reagents were used without further purification, unless otherwise stated. The anhydrous solvents were purchased in anhydrous form (dichloromethane, dimethylsulfoxide, N,N-dimethylformamide, tetrahydrofuran, etc.) and all other solvents used were reagent grade or HPLC grade.

Nuclear magnetic resonance spectra were recorded at ambient temperature (unless otherwise stated) on a 400 MHz spectrometer: Bruker Avance 400 Ultrashield (400 MHz for ¹H, 100 MHz for ¹³C). All values for proton chemical shifts are reported in parts per million (4) and are referenced to the deuterated proton in CDCh (δ 7.26) or deuterated protons DMSO-d₆ (δ 2.50) or deuterated protons D₂O (δ 4.79). All values for carbon chemical shifts are reported in parts per million ((δ) and are referenced to the carbon resonances in CDCh (δ 77.0) or DMSO-d₆ (δ 39.52). NMR data are represented as follows: chemical shift, multiplicity (s=singlet, d=doublet, t=triplet, q=quartet, quin=quintet, m=multiplet, br=broad, dd=doublet of doublet), coupling constant=J (Hz=Hertz), and integration.

Low-resolution mass spectra were collected on a Bruker Amazon SL (ESI) or Thermo Fisher LCQ advantage (ESI) spectrometer.

Glassware and stir bars were generally dried in an oven at 140° C. for at least 12 h and then cooled under N₂ atmosphere prior to use where applicable. Vials were fitted with crimp top septa under a positive pressure of N₂, unless otherwise noted. All other reactions were performed in round-bottom flasks sealed with rubber septa. Plastic syringes or glass pipets were used to transfer liquid reagents. Reactions were stirred magnetically using teflon-coated, magnetic stir bars. Analytical thin-layer chromatography (TLC) was performed using aluminum plates pre-coated with 0.25 mm of 230-400 mesh silica gel impregnated with a fluorescent indicator (254 nm). TLC plates were visualized by exposure to ultraviolet light and/or exposure to KMnO₄ staining solution. Organic solutions were concentrated under reduced pressure using a rotary evaporator KNF RC 600 and Heidolph Hei-VAP.

Flash column chromatography was performed with pre-packed FLASH silica gel columns and using Biotage Isolera™ One and Biotage Isolera™ SL (big scale) flash purification systems.

Trifluroacetic acid (TFA) content was determined using evaporative light scattering detector (ELSD LT-II, Shimadzu) and an Acclaim Trinity PI ion exchange column (3.0 μm, 3.0×150 mm). The quantification of TFA was performed in triplicate for each sample and calculated from a 7-point calibration curve.

The pH value of a solution was measured at room temperature using a pH meter WTW Inolab 7310 with SenTix® mic-D electrodes.

HPLC was performed using a Shimadzu Nexera XR HPLC system equipped with a SPD-M20A photodiode array detector.

Lyophilization was carried out using Martin Christ Alpha 2-4 LSCplus.

Centrifugation was carried out using Eppendorf centrifuge 5810 R, refrigerated, with Rotor A-4-81.

### HPLC Methods

HPLC method 1: HPLC method for reaction monitoring and purity. Column: Phenomenex Kinetex Polar C18 (150×4.6 mm, 2.6 μm, 100 Å), gradient: mobile phase A: water (0.1% TFA), mobile phase B: acetonitrile (0.1% TFA). Elution gradient of phase B: 0-3.5 min: 30%, 3.5-20 min: 30-80%, 20-22 min: 80-100%, 22-24 min: 100%, 24-27 min: 100-30%, 27-30 min: 30%, 30 minutes: method end, flow rate=1.0 mL/min.

HPLC method 2: HPLC method for reaction monitoring and purity. Column: Phenomenex Kinetex Polar C18 (150×4.6 mm, 2.6 μm, 100 Å), gradient mobile phase A: water (0.1% TFA), mobile phase B: acetonitrile (0.1% TFA). Elution gradient of phase B: 0-3.5 min: 5%, 3.5-20 min: 5-40%, 20-22 min: 40-65%, 22-24 min: 65%, 24-27 min: 65-5%, 27-30 min: 5%, 30 minutes: method end, flow rate=1.0 mL/min.

HPLC method 3: HPLC method for reaction monitoring and purity. Column: Phenomenex Kinetex Polar C18 (150×4.6 mm, 2.6 μm, 100 Å), gradient mobile phase A: water (0.1% TFA), mobile phase B: acetonitrile (0.1% TFA). Elution gradient of phase B: 0-5.5 min: 1%, 5.5-20 min: 1-40%, 20-22 min: 40-65%, 22-24 min: 65%, 24-27 min: 65-1%, 27-30 min: 1%, 30 minutes: method end, flow rate=1.0 mL/min.

HPLC Method 4: HPLC method for reaction monitoring and purity. Column: Phenomenex Kinetex Polar C18 (150×4.6 mm, 2.6 μm, 100 Å), gradient mobile phase A: water (0.1% TFA), mobile phase B: acetonitrile (0.1% TFA). Elution gradient of phase B: 0-1.5 min: 1%, 1.5-8 min: 1-65%, 8-10 min: 65-95%, 10-13 min: 95%, 13-14 min: 95-1%, 14-16 min: 1%, 15 minutes: method end, flow rate=1.0 mL/min.

HPLC method 5: HPLC method for reaction monitoring and purity. Column: Phenomenex reverse phase Kinetex Polar C18 column (150×4.6 mm, 2.6 μm), gradient mobile phase A: water (0.1% TFA), mobile phase B: acetonitrile (0.1% TFA). Elution gradient of phase B: 0-1.5 min: 5%, 1.5-8 min: 5-65%, 8-13 min: 65-95%, 13-15 min: 95-5%, 15 minutes: method end, flow rate=1.0 mL/min.

HPLC method 6: HPLC method for reaction monitoring and purity. Column: Phenomenex reverse phase Kinetex Polar C18 column (150×4.6 mm, 2.6 μm), gradient mobile phase A: water (0.1% TFA), mobile phase B: acetonitrile (0.1% TFA). Elution gradient of phase B: 0-1.5 min: 15%, 1.5-8 min: 15-70%, 8-10 min: 70-95%, 10-13 min: 95%, 13-15 min: 95-15%, flow rate=1.0 mL/min.

HPLC method 7: HPLC method for reaction monitoring and purity. Column: Phenomenex reverse phase Kinetex Polar C18 column (150×4.6 mm, 2.6 μm), gradient mobile phase A: water (0.1% TFA), mobile phase B: acetonitrile (0.1% TFA). Elution gradient of phase B: 0-1.5 min: 5%, 1.5-20 min: 5-65%, 20-22 min: 65-95%, 24-30 min: 95-5%, 30 minutes: method end, flow rate=1.0 mL/min.

HPLC method 8: HPLC method for radiochemical monitoring and purity. Column: Phenomenex reverse phase Kinetex Polar C18 column (150×4.6 mm, 2.6 μm), gradient mobile phase A: water (0.1% TFA), mobile phase B: acetonitrile (0.1% TFA). Elution gradient of phase B: 0-1.5 min: 5%, 1.5-20 min: 5-40%, 20-22 min: 40-65%, 22-24 min: 65%, 24-25 min: 65-5%, 25-30 min: 5%. 30 minutes: method end, flow rate=1.0 mL/min. Column oven: 37° C.

HPLC Method 9: HPLC method for serum albumin binding. Column: Phenomenex Aeris WP C18 (250×4.6 mm, 3.6 μm, widepore), gradient: mobile phase A: Water with 0.1% TFA, mobile phase B: Acetonitrile with 0.1% TFA. Elution gradient of phase B: 0-1.5 min: 5%, 1.5-20.0 min: 5-60%, 20-22.0 min: 60-85%, 22-24.0 min: 85-85%, 24-27.0 min: 85-5%, 30 minutes: method end, flow rate=1.0 mL/min. Column oven: 37° C.

### LCMS Method

LC-MS method: Column: Phenomenex Kinetex Polar C18 (150×2.1 mm, 2.6 μm, 100 Å), gradient: mobile phase A: 0.1% FA in H₂O: acetonitrile, mobile phase B: 0.1% FA in CH₃CN. Elution gradient of phase B: 0-1.0 min: 15%, 1.0-10.0 min: 15-100%, 10.0-11.5 min: 100%, 11.50-12.50 min: 15%, 12.50-15.0 min: 15%, 15.0 minutes: method end, 15 minutes: method end, flow rate=0.4 mL/min.

### TFA (Trifluoracetic Acid) Quantification

TFA content of the purified chelates was determined using an evaporative light scattering detector (ELSD LT-II, Shimadzu, gain set to 5, T=30° C.) and an Acclaim Trinity PI ion exchange column (3.0 μm, 3.0×150 mm). Mobile phase A: 90% acetonitrile/10% Millipore water. Mobile phase B: 0.2 M ammonium acetate pH 4.3. Elution gradient of phase B: 0-4.0 min: 25%, 4.0-10.0 min: 25-90%, 10.0-13.0 min: 90%, 13.0-15.0 min: 90-25%, 15.0-20.0 min: 25%, 20.0 minutes: method end. Flow rate=0.6 mL/min. Injection volume: 30 μL. The quantification of TFA was performed in triplicate for each sample (dissolved in 0.02 M HCl) and calculated from a 7-point calibration curve (calibration range 1.15 mM to 6.49 mM). A 6.5 mM TFA stock solution is injected at least 4 times to prime the column before running the batch.

### Radiochemical Labeling and Purity

Preparation of lyophilized formulation: C4-DTPA was dissolved in the lyophilization buffer at a final concentration of 0.09 mg/mL. The lyophilization buffer contained 2 mg/mL of gentisic acid, 10 mg/mL of inositol, 5.6 mg/mL of sodium citrate and 0.4 mg/mL of citric acid. The dissolved chelate was sterile-filtered using an Acrodisc Fluorodyne II syringe filter (0.2 μm). The sterile solutions were pipetted into lyophilization vials which were then partially sealed using a rubber stopper. The vials were subsequently placed into the freeze dryer for lyophilization. The vials were gradually frozen over a period of 9 hours to reach a final temperature of −40° C. Main drying was initiated at the end of the freezing cycle, main drying was performed between −40° C. and −20° C. over a total period of 52 hours (vacuum 0.08 mbar). At the end of the main drying cycle, final drying was initiated. Final drying was performed for 12 hours at 25° C. (vacuum 0.08 mbar). Upon completion of the final drying the vials were sealed under vacuum.

Radiolabeling and quality control: The radiolabeling of C4-DTPA was performed by adding 42.4±7.6 MBq (C4-DTPA) of ¹¹¹InCl₃ in HCl solution (MAP Medical, Finland) into vials containing lyophilized test compound. The vials were vortexed until a clear solution was reached and incubated for 30 minutes at +37° C. The radiolabeling was performed in triplicate.

The radiochemical purity of the radiolabeled product ¹¹¹In-C4-DTPA was measured with reverse phase C18 (Agilent 1260 Infinity II chromatography system, method 8). The sample was diluted 1:10 (v:v) with water and 10 μl was injected to the HPLC system.

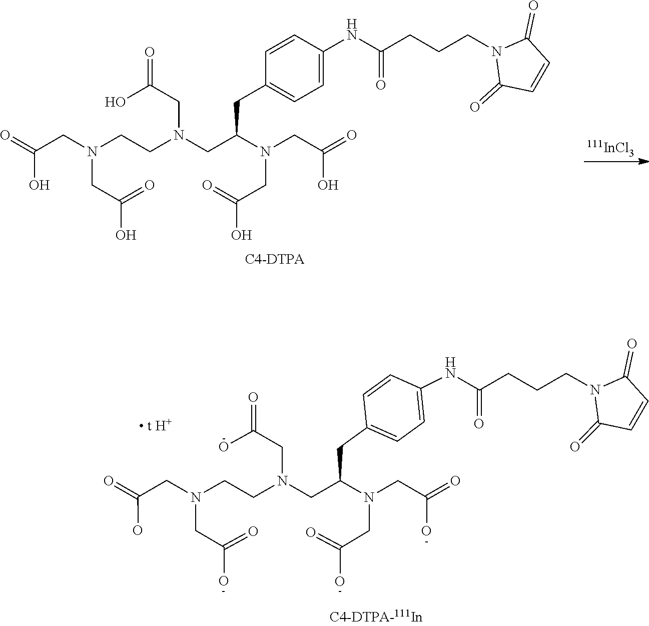 [SMILES: O=C(O)CN(CCN(CC(=O)O)C[C@@H](Cc1ccc(NC(=O)CCCN2C(=O)C=CC2=O)cc1)N(CC(=O)O)CC(=O)O)CC(=O)O ; "111InCl3" ; O=C(O)CN(CCN(CC(=O)O)C[C@@H](Cc1ccc(NC(=O)CCCN2C(=O)C=CC2=O)cc1)N(CC(=O)O)CC(=O)O)CC(=O)O]

Chromatography: Infinity II 1260 HPLC system (Agilent Inc., Santa Clara, Calif., USA) consisting of a G711A solvent delivery system, G7129A thermostated autosampler, G7114A UV detector, G1364C analytical fraction collector and PosiRAM radio-detector (LabLogic Systems Ltd.). The HPLC system was controlled with LAURA control and analysis software (LabLogic Systems Ltd., U.K.). Analytes were separated with a gradient run on a RP-HPLC column, Kinetex Polar C18 (4.6 mm×150 mm with a guard column). HPLC method 8 was used.

### Example 2

### Synthetic Examples

Compounds 3a-d shown in Scheme 5 were prepared as described below.

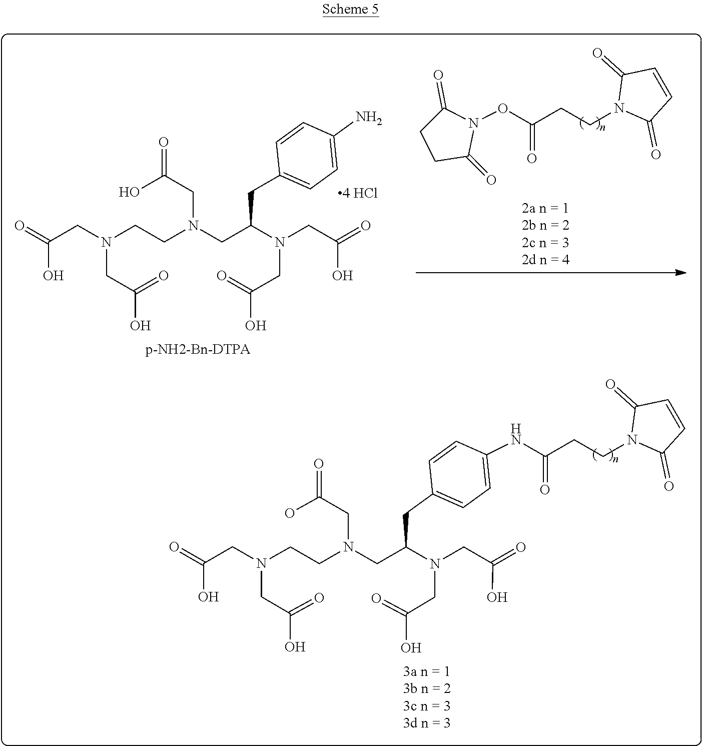

General Procedure for the Synthesis of Compounds 3a-3d

p-NH₂—Bn-DTPA*4 HCl 1 (1.0 eq.) was mixed with anhydrous acetonitrile (2 mL/100 mg) in a 12 mL Falcon tube. DIPEA (11 eq.) was added and the mixture was dissolved using ultrasonication. A solution of the maleimido A-hydroxysuccinimide ester 2a-2d (2 eq.) in dry MeCN (2 mL) was then added to the DTPA solution, and the mixture was stirred at room temperature, and the reaction progress was followed by HPLC. After 18 h, acetic acid (11 eq.) was added to neutralize the base and to stop the reaction. The product was precipitated from MTBE (8 mL), and the obtained precipitate was centrifuged (4 min, 4000 rpm) and purified by RP-FC. For RP-FC purification, the precipitate was dissolved in a mixture of 600 μL MeCN and 600 μL water. The resulting solution was diluted with water to a volume of 6 mL (10% MeCN). The solution was purified by RP flash chromatography on a Biotage Isolera™ One flash purification System, with a pre-packed SNAP Ultra C18 30 g column, using step gradients of water containing 0.1% TFA and acetonitrile containing 0.1% TFA. Product fractions were analyzed by HPLC (method 2, 220 nm), pure fractions combined and lyophilized to give a white fluffy solid 3a-3d.

Yield and Characterization Data for 3a-3d

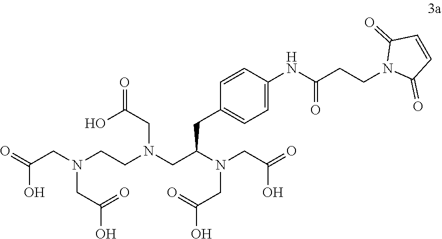 [SMILES: O=C(O)CN(CCN(CC(=O)O)C[C@@H](Cc1ccc(NC(=O)CCN2C(=O)C=CC2=O)cc1)N(CC(=O)O)CC(=O)O)CC(=O)O]

(R)-2,2′-((2-((2-(bis(carboxymethyl)amino)-3-(4-(3-(2,5-dioxo-2,5-dihydro-1H-pyrrol-1-yl)propanamido)phenyl)propyl)(carboxymethyl)amino)ethyl)azanediyl)diacetic acid 3a, was prepared as described above. Yield: 35 mg (35%) (TFA content: 0.75±0.03 mol. eq.). HPLC (method 2, 220 nm) ≥95%. LRMS-ESI (m/z) calcd. for: C₂₈H₃₆N₅O₁₃ [M+H]⁺: 650.23. Found: 650.08. ¹H NMR (400 MHz, D₂O) δ 7.35 (d, J=8.4 Hz, 2H), 7.27 (d, J=8.4 Hz, 2H), 6.84 (s, 2H), 3.99-3.71 (m, 12H), 3.68-3.58 (m, 1H), 3.47-3.10 (m, 7H), 2.77-2.62 (m, 3H). ¹³C NMR (101 MHz, D₂O, as a TFA salt, the signals of TFA were not included) 172.67, 172.59, 172.20, 171.28, 170.66, 135.78, 134.43, 133.14, 129.88, 122.32, 62.01, 55.52, 54.48, 54.16, 51.24, 49.88, 35.50, 34.42, 31.92. ¹⁹F NMR (376 MHz, D₂O) δ −75.59.

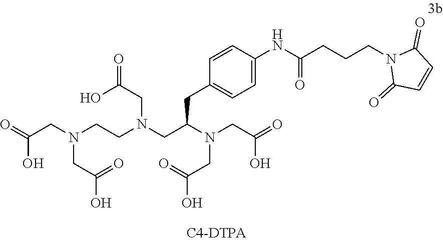 [SMILES: O=C(O)CN(CCN(CC(=O)O)C[C@@H](Cc1ccc(NC(=O)CCCN2C(=O)C=CC2=O)cc1)N(CC(=O)O)CC(=O)O)CC(=O)O]

(R)-2,2′-((2-((2-(bis(carboxymethyl)amino)-3-(4-(4-(2,5-dioxo-2,5-dihydro-1H-pyrrol-1-yl)butanamido)phenyl)propyl)(carboxymethyl)amino)ethyl)azanediyl)diacetic acid 3b, C4-DTPA, was prepared as described above. Yield: 114 mg (22%) (TFA content: 1.29±0.22 mol. eq.). HPLC (method 2, 220 nm) ≥95%. %. LRMS-ESI (m/z) calcd. for: C₂₉H₃₆N₅O₁₃ [M−H]⁻: 662.23. Found: 662.33. ¹H NMR (400 MHz, D₂O) δ 7.34 (d, J=8.1 Hz, 2H), 7.21 (d, J=8.2 Hz, 2H), 6.71 (s, 2H), 4.00-3.65 (m, 10H), 3.64-3.54 (m, 1H), 3.51 (t, J=6.5 Hz, 2H), 3.45-3.00 (m, 7H), 2.65 (dd, J=13.9, 9.2 Hz, 1H), 2.36 (t, J=7.0 Hz, 2H), 1.92 (p, J=6.8 Hz, 2H). ¹³C NMR (101 MHz, D₂O, as a TFA salt, the signals of TFA were not included) δ 173.92, 173.06, 172.77, 170.93, 170.56, 136.05, 134.27, 132.76, 129.80, 121.92, 62.00, 55.26, 54.63, 53.99, 52.62, 51.10, 49.95, 36.95, 33.47, 31.94, 23.33. ¹⁹F NMR (376 MHz, D₂O) δ −75.65.

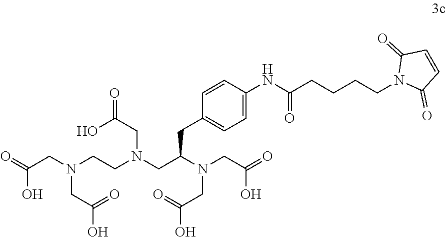 [SMILES: O=C(O)CN(CCN(CC(=O)O)C[C@@H](Cc1ccc(NC(=O)CCCCN2C(=O)C=CC2=O)cc1)N(CC(=O)O)CC(=O)O)CC(=O)O]

(R)-2,2′-((2-((2-(bis(carboxymethyl)amino)-3-(4-(5-(2,5-dioxo-2,5-dihydro-1H-pyrrol-1-yl)pentanamido)phenyl)propyl)(carboxymethyl)amino)ethyl)azanediyl)diacetic acid 3c was synthesized as described above. Yield: 47.0 mg (22%) (TFA content: 1.05±0.01 mol. eq.). HPLC (method 5, 220 nm) >95%. LRMS-ESI (m/z) calcd. for C₃₀K₃₉N₅O₁₃ [M+H]⁺: 678.26. Found: 678.23. TFA content (per mol of compound): 1.05 mol eq. ¹H NMR (400 MHz, D₂O) δ 7.36 (d, J=8.4 Hz, 2H), 7.21 (d, J=8.4 Hz, 2H), 6.75 (s, 2H), 4.00-3.67 (m, 10H), 3.60-3.53 (m, 1H), 3.45 (t, J=6.2 Hz, 2H), 3.38-3.02 (m, 7H), 2.63 (dd, J=13.6, 9.5 Hz, 1H), 2.35 (t, J=6.5 Hz, 2H), 1.64-1.49 (m, 4H); ¹³C NMR (101 MHz, D₂O, as a TFA salt, the signals of TFA were not included) δ 174.97, 173.18, 172.85, 170.82, 170.61, 136.08, 134.20, 132.82, 129.82, 122.04, 61.99, 55.15, 54.50, 54.03, 52.52, 50.98, 49.98, 37.13, 35.72, 31.90, 27.00, 22.39.

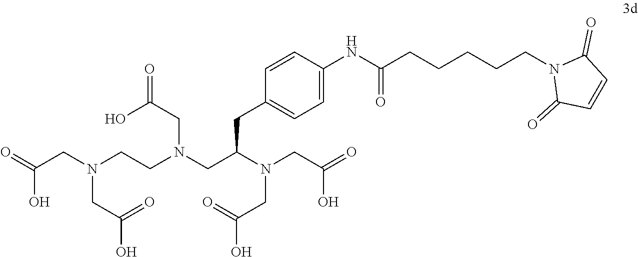 [SMILES: O=C(O)CN(CCN(CC(=O)O)C[C@@H](Cc1ccc(NC(=O)CCCCCN2C(=O)C=CC2=O)cc1)N(CC(=O)O)CC(=O)O)CC(=O)O]

(R)-2,2′-((2-((2-(bis(carboxymethyl)amino)-3-(4-(6-(2,5-dioxo-2,5-dihydro-1H-pyrrol-1-yl)hexanamido)phenyl)propyl)(carboxymethyl)amino)ethyl)azanediyl)diacetic acid 3d was synthesized as described above. Yield: 23 mg (21%) (TFA content: 1.17±0.10 mol. eq.). HPLC (method 2, 220 nm) ≥95%. LRMS-ESI (m/z) calcd. for: C₃₁H₄₂N₅O₁₃ [M+H]⁺: 692.28. Found: 692.26. ¹H NMR (400 MHz, DMSO-d₆) δ 9.83 (s, 1H), 7.52 (d, J=8.5 Hz, 2H), 7.17 (d, J=8.5 Hz, 2H), 7.00 (s, 2H), 4.52-4.35 (m, 2H), 3.59-3.31 (m, 14H), 3.17 (d, J=11.0 Hz, 1H), 3.05-2.97 (m, 2H), 2.93 (dd, J=13.5, 5.1 Hz, 1H), 2.48-2.44 (m, 1H), 2.26 (t, J=7.4 Hz, 2H), 1.62-1.53 (m, 2H), 1.54-1.46 (m, 2H), 1.24 (p, J=7.7, 7.2 Hz, 2H). ¹³C NMR (101 MHz, DMSO d₆, as a TFA salt, the signals of TFA were not included) δ 173.97, 172.43, 171.13, 171.01, 167.77, 137.81, 134.48, 132.23, 129.33, 119.17, 59.03, 55.40, 53.89, 53.28, 52.07, 51.46, 48.72, 37.00, 36.16, 33.21, 27.82, 25.85, 24.63.

Compounds 6a-d and 7a-d shown in Scheme 6 were prepared as described below.

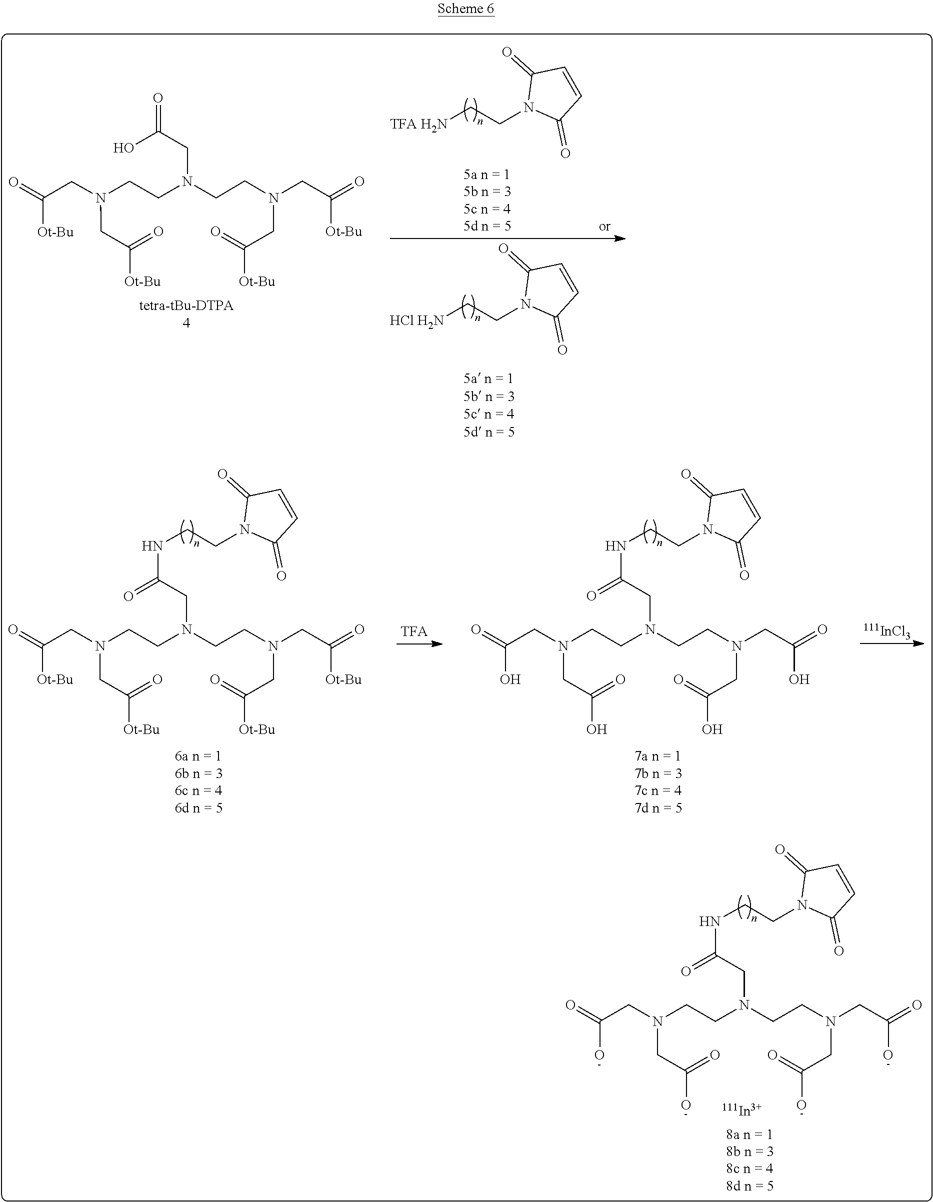

### General Procedure for the Synthesis of Compounds 6 and 7

Step 1: To a cold (−15° C.) solution of tetra-tBu-DTPA 4 (1.00 eq) in THF (1000 μL/150 mg) was added DIPEA (2.00 eq) and isobutyl chloroformate (1.10 eq.). After 10 min, a THF suspension (3000 μL) of the amino maleimide derivative 5a-d or 5a′-d' (1.00 eq.) was added dropwise. The mixture was stirred for 30 min at −15° C. and then allowed to warm to room temperature and stirred for 12 h. The reaction mixture was concentrated under reduced pressure and the residue was purified by flash chromatography on a Biotage Isolera™ One flash purification system, with a pre-packed SNAP Ultra 25 g column, using a gradient from 0% to 10% methanol in chloroform to give the title compound 6a-d as a brown viscous oil.

Step 2: A mixture of trifluoroacetic acid (220 eq.) and anisole (7.75 eq.) was added to a flask containing the product from step 1, 6a-d (1.00 eq.). The mixture was stirred at 25° C. for 24 h. The reaction was poured dropwise in a falcon tube containing 30 mL diethyl ether. A white precipitate formed immediately. The tube was left to stand in the freezer at −20° C. for 1 h. The precipitate was centrifuged (4000 rpm, 5 min), washed with diethyl ether (5 mL) and dried under high vacuum for 20 h to afford the title compound 7a-d as a white microcrystalline solid.

### Yield and Characterization Data for Compounds 6a-d and 7a-d

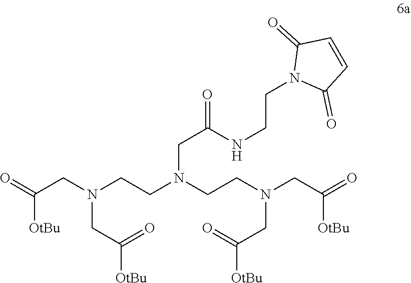 [SMILES: CC(C)(C)OC(=O)CN(CCN(CCN(CC(=O)OC(C)(C)C)CC(=O)OC(C)(C)C)CC(=O)NCCN1C(=O)C=CC1=O)CC(=O)OC(C)(C)C]

Tetra-tert-butyl 2,2′,2″,2′″-((((2-((2-(2,5-dioxo-2,5-dihydro-1H-pyrrol-1-yl)ethyl)amino)-2-oxoethyl)azanediyl)bis(ethane-2,1-diyl))bis(azanetriyl))tetraacetate 6a was synthesized as described above. Yield: 147 mg (83%), HPLC (method 1, 220 nm) >95%. LRMS-ESI (m/z) calcd. for C₃₆H₆₂N₅O₁₁ [M+H]⁺: 740.44. Found: 740.53.

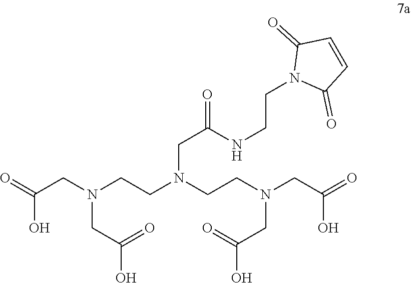 [SMILES: O=C(O)CN(CCN(CCN(CC(=O)O)CC(=O)O)CC(=O)NCCN1C(=O)C=CC1=O)CC(=O)O]

2,2′,2″,2′″-((((2-((2-(2,5-dioxo-2,5-dihydro-1H-pyrrol-1-yl)ethyl)amino)-2-oxoethyl)azanediyl)bis(ethane-2,1-diyl))bis(azanetriyl))tetraacetic acid 7a was synthesized as described above. Yield: 73 mg (89%) (TFA content: 1.13±0.20 mol. eq.). HPLC (method 3, 220 nm) >97%. LRMS-ESI (m/z) calcd. for C₂₀H₃₀N₅O₁₁ [M+H]⁺: 516.19. Found: 516.20. ¹H NMR (400 MHz, Deuterium Oxide) δ 6.86 (s, 2H), 4.09 (s, 8H), 3.74-3.62 (m, 2H), 3.54 (d, J=3.5 Hz, 2H), 3.49 (t, J=6.4 Hz, 4H), 3.44-3.35 (m, 2H), 3.18 (t, 7=6.4 Hz, 4H). ¹³C NMR (101 MHz, D₂O, as a TFA salt, the signals of TFA were not included) δ 173.02, 171.10, 169.92, 134.44, 55.63, 55.38, 52.46, 50.12, 38.06, 36.97.

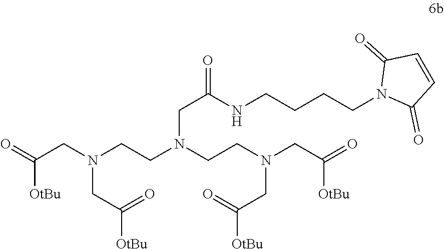 [SMILES: CC(C)(C)OC(=O)CN(CCN(CCN(CC(=O)OC(C)(C)C)CC(=O)OC(C)(C)C)CC(=O)NCCCCN1C(=O)C=CC1=O)CC(=O)OC(C)(C)C]

The amine 5b or 5b′ was synthesized according to a previously described procedure (Horstmann et al., Bioorganic Chemistry, 57:155-161 (2014)). Tetra-tert-butyl 2,2′,2″,2′″-((((2-((4-(2,5-dioxo-2,5-dihydro-1H-pyrrol-1-yl)butyl)amino)-2-oxoethyl)azanediyl)bis(ethane-2,1-diyl))bis(azanetriyl))tetraacetate 6b was synthesized as described above. Yield: 99 mg (80%), HPLC (method 1, 220 nm)=95%. LRMS-ESI (m/z) calcd. for C₃₈H₆₆N₅O₁₁ [M+H]⁺: 768.48. Found: 768.58.

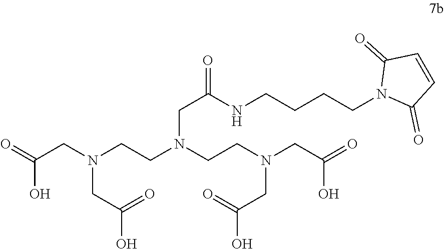 [SMILES: O=C(O)CN(CCN(CCN(CC(=O)O)CC(=O)O)CC(=O)NCCCCN1C(=O)C=CC1=O)CC(=O)O]

2,2′,2″,2′″-((((2-((4-(2,5-dioxo-2,5-dihydro-1H-pyrrol-1-yl)butyl)amino)-2-oxoethyl)azanediyl)bis(ethane-2,1-diyl))bis(azanetriyl))tetraacetic acid 7b was synthesized as described above. Yield: 78 mg (99%) (TFA content: 1.07±0.20 mol. eq.). HPLC (method 2, 220 nm) >98%. LRMS-ESI (m/z) calcd. for C₂₂H₃₄N₅O₁₁ [M+H]⁺: 544.22. Found: 544.21. ¹H NMR (400 MHz, D₂O) δ 6.80 (s, 2H), 3.98 (s, 8H), 3.55-3.40 (m, 8H), 3.24-3.08 (m, 6H), 1.67-1.41 (m, 4H). ¹³C NMR (101 MHz, Deuterium Oxide) δ 173.35, 170.85, 170.02, 134.30, 56.08, 55.88, 52.35, 49.84, 38.74, 37.18, 25.55, 25.04.

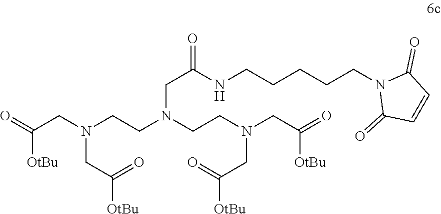 [SMILES: CC(C)(C)OC(=O)CN(CCN(CCN(CC(=O)OC(C)(C)C)CC(=O)OC(C)(C)C)CC(=O)NCCCCCN1C(=O)C=CC1=O)CC(=O)OC(C)(C)C]

Tetra-tert-butyl 2,2′,2″,2′″-((((2-((5-(2,5-dioxo-2,5-dihydro-1H-pyrrol-1-yl)pentyl)amino)-2-oxoethyl)azanediyl)bis(ethane-2,1-diyl))bis(azanetriyl))tetraacetate 6c was synthesized as described above. Yield: 120 mg (47%), HPLC (method 1, 220 nm) ≥95%. LRMS-ESI (m/z) calcd. for: C₃₉H₃₉N₅O₁₁ [M+H]⁺: 782.49. Found: 782.60. ¹H NMR (400 MHz, CDCl₃) δ 8.11 (s, 1H), 6.66 (s, 2H), 3.49 (t, J=7.2 Hz, 2H), 3.40 (s, 8H), 3.21 (q, J=6.9 Hz, 2H), 3.09 (s, 2H), 2.76 (s, 4H), 2.60 (s, 4H), 1.63-1.50 (m, 4H), 1.43 (s, 36H), 1.35-1.22 (m, 2H). ¹³C NMR (101 MHz, CDCl₃) δ 170.91, 170.71, 170.66, 134.15, 81.14, 58.77, 55.97, 53.87, 52.21, 39.10, 37.88, 29.36, 28.40, 28.28, 24.36.

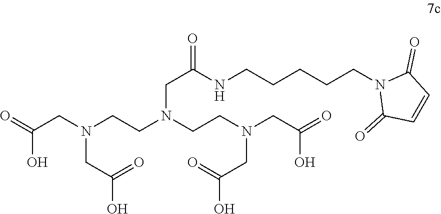 [SMILES: O=C(O)CN(CCN(CCN(CC(=O)O)CC(=O)O)CC(=O)NCCCCCN1C(=O)C=CC1=O)CC(=O)O]

2,2′,2″,2′″-((((2-((5-(2,5-dioxo-2,5-dihydro-1H-pyrrol-1-yl)-pentyl)-amino)-2-oxoethyl)azanediyl)bis(ethane-2,1-diyl))bis(azanetriyl))tetraacetic acid 7c was synthesized as described above. Yield: 65.0 mg (89%) (calculated as TFA salt. TFA content: 0.81±0.16 mol. eq.) HPLC (method 2, 220 nm) ≥95%. LRMS-ESI (m/z) calcd. for: C₂₃H₃₆N₅O₁₁ [M+H]⁺: 558.24. Found: 558.24. ¹H NMR (400 MHz, DMSO-d₆) δ 8.38 (s, 1H), 7.00 (s, 2H), 3.92 (s, 2H), 3.47 (s, 8H), 3.38 (t, J=7.1 Hz, 2H), 3.20 (s, 4H), 3.13-3.04 (m, 2H), 2.97 (s, 4H), 1.53-1.46 (m, 2H), 1.47-1.37 (m, 2H), 1.27-1.15 (m, 2H). ¹³C NMR (101 MHz, DMSO-d₆, as a TFA salt, the signals of TFA were not included) δ 173.30, 173.23, 171.57, 134.93, 54.96, 54.81, 52.61, 49.69, 38.96, 37.41, 28.78, 28.11, 23.

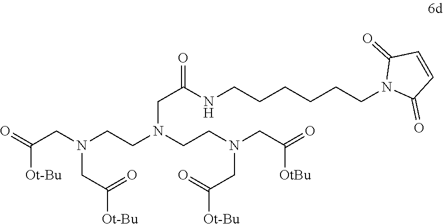 [SMILES: CC(C)(C)OC(=O)CN(CCN(CCN(CC(=O)OC(C)(C)C)CC(=O)OC(C)(C)C)CC(=O)NCCCCCCN1C(=O)C=CC1=O)CC(=O)OC(C)(C)C]

The amine 5d or 5d′ was synthesized according to a previously described procedure (Horstmann et al., Bioorganic Chemistry, 57:155-161 (2014)). Tetra-tert-butyl 2,2′,2″,2′″-((((2-((6-(2,5-dioxo-2,5-dihydro-1H-pyrrol-1-yl)hexyl)amino)-2-oxoethyl)azanediyl)bis(ethane-2,1-diyl))bis(azanetriyl))tetraacetate 6d was synthesized as described above. Yield: 21 mg (16%), HPLC analysis >94% (method 1, 220 nm), LRMS-ESI (m/z) calcd. for: C₄₀H₇₀N₅O₁₁ [M+H]⁺: 796.50. Found: 796.67, ¹H NMR (300 MHz, CDCl₃) δ 8.05 (brs, 1H), 6.65 (s, 2H), 3.47 (t, 2H, J=13 Hz), 3.38 (s, 8H), 3.21-3.16 (q, 2H, J=6.8 Hz), 3.08 (brs, 2H), 2.77-2.73 (m, 4H), 2.61-2.57 (m, 4H), 1.57-1.22 (m, 50H). ¹³C NMR (101 MHz, CDCl₃) δ 171.84, 170.90, 170.61, 134.12, 81.10, 58.78, 55.94, 53.87, 52.20, 39.14, 37.88, 29.70, 28.57, 28.45, 28.26, 28.05, 26.65, 26.54.

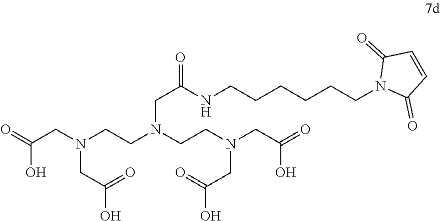 [SMILES: O=C(O)CN(CCN(CCN(CC(=O)O)CC(=O)O)CC(=O)NCCCCCCN1C(=O)C=CC1=O)CC(=O)O]

2,2′,2″,2′″-((((2-((6-(2,5-dioxo-2,5-dihydro-1H-pyrrol-1-yl)hexyl)amino)-2-oxoethyl)azanediyl)bis(ethane-2,1-diyl))bis(azanetriyl))tetraacetic acid 7d was synthesized as described above. Yield: 5.0 mg, 33%, (calculated as TFA salt. TFA content: 0.92±0.17 mol. eq.) HPLC analysis: purity 97.69% (method 1, 220 nm), LRMS-ESI (m/z) calcd. for: C₂₄H₃₈N₅O₁₁ [M+H]⁺: 572.25. Found: 572.29, ¹H NMR (300 MHz, CDCl₃) δ 6.83 (s, 2H), 4.01 (s, 8H), 3.59 (s, 2H), 3.52-3.47 (m, 6H), 3.23-3.17 (m, 6H), 1.60-1.47 (m, 4H), 1.36-1.18 (m, 4H). ¹³C NMR (101 MHz, CDCl₃) δ 173.41, 170.42, 169.54, 134.24, 55.60, 51.93, 50.58, 39.30, 37.44, 28.07, 27.43, 25.48, 25.40.

Compounds 10b-d were synthesized as shown Scheme 7 and according to the general procedure as described below.

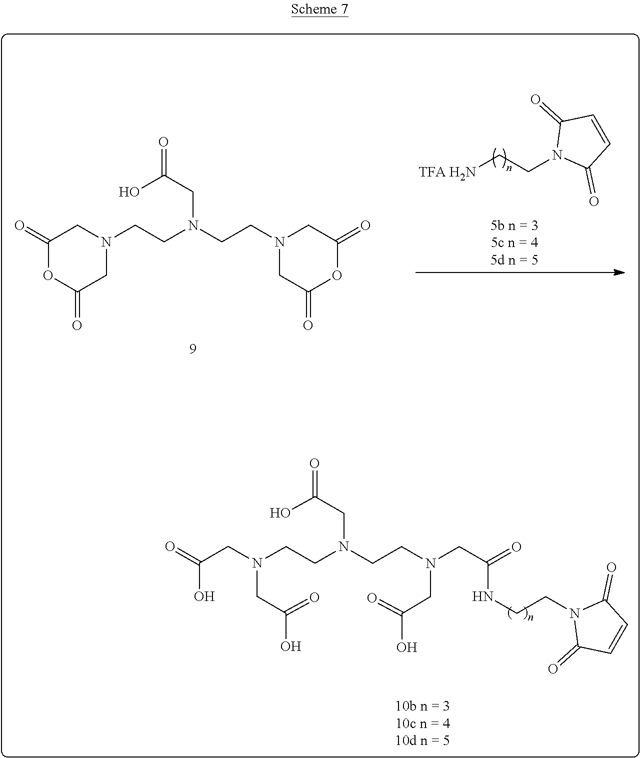

General Procedure for the Synthesis of Compounds 10b-10d

To a suspension of DTPA-dianhydride 9 (1 eq., 560 μmol, 200 mg) in anhydrous DMF (3 mL) in a 15 mL Falcon tube the linker trifluoroacetate 5b-5d (0.6 eq.) in anhydrous DMF (1 mL) was added in three portions over the course of 2 h (333 μL/h), the suspension was vortexed for a few seconds and the reaction was left stirring for 3 h. Water (4 mL) was added to the suspension. The obtained solution was directly injected into a reversed-phase C18 cartridge and purified with flash chromatography. The purification was carried out using a step gradient, from 100% water (0.1% TFA) to 100% acetonitrile (0.1% TFA) over 10 column volumes. Fractions containing the product were combined and lyophilized to achieve the target compound as a fluffy white solid.

Yield and Characterization Data for 10b-10d

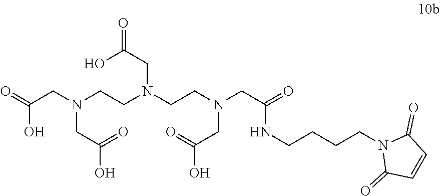 [SMILES: O=C(O)CN(CCN(CC(=O)O)CC(=O)O)CCN(CC(=O)O)CC(=O)NCCCCN1C(=O)C=CC1=O]

2-({2-[bis(carboxymethyl)amino]ethyl}({2-[(carboxymethyl)({[4-(2,5-dioxo-2,5-dihydro-1H-pyrrol-1-yl)butyl]carbamoyl}methyl)amino]ethyl})amino)acetic acid 10b was prepared as described above. Yield: 44 mg (35% yield referring to the linker) (calculated as TFA salt. TFA content: 1.83±0.23 mol. Eq.), HPLC (method 2, 220 nm) 98%. LRMS-ESI (m/z) calcd. for C₂₂H₃₃N₅O₁₁ [M+H]⁺: 544.53. Found: 544.27. ¹H NMR (400 MHz, D₂O) δ 6.82 (s, 2H), 4.11 (s, 4H), 4.04 (s, 2H), 4.03 (s, 2H), 3.72 (s, 2H), 3.54-3.45 (m, 6H), 3.27-3.20 (m, 6H), 1.62-1.46 (m, 4H); ¹³C NMR (101 MHz, D₂O, as a TFA salt, the signals of TFA were not included) δ 173.30, 172.91, 169.91, 166.51, 134.28, 56.27, 55.60, 55.54, 53.77, 52.56, 50.03, 49.93, 38.90, 37.08, 25.43, 24.96.

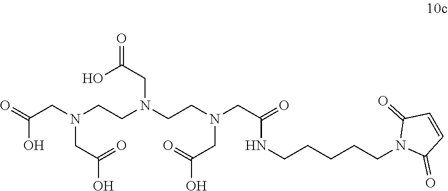 [SMILES: O=C(O)CN(CCN(CC(=O)O)CC(=O)O)CCN(CC(=O)O)CC(=O)NCCCCCN1C(=O)C=CC1=O]

2-{[2-({2-[bis(carboxymethyl)amino]ethyl}(carboxymethyl)amino)ethyl]({[5-(2,5-dioxo-2,5-dihydro-1H-pyrrol-1-yl)pentyl]carbamoyl}methyl)amino}acetic acid 10c was prepared as described above. Yield: 87 mg (36% yield referring to the linker) (calculated as TFA salt. TFA content: 1.46±0.25 mol. Eq.), HPLC (method 2, 220 nm) 98%. LRMS-ESI m/z) calcd. for C₂₃H₃₅N₅O₁₁ [M+H]⁺: 558.56. Found: 558.26. ¹H NMR (400 MHz, D₂O) δ 6.82 (s, 2H), 4.08 (s, 4H), 4.02 (s, 2H), 4.00 (s, 2H), 3.73 (s, 2H), 3.53-3.45 (m, 6H), 3.26-3.21 (m, 6H), 1.60-1.49 (m, 4H), 1.30-1.22 (m, 2H); ¹³C NMR (101 MHz, D₂O, as a TFA salt, the signals of TFA were not included) δ 173.38, 172.85, 170.12, 170.05, 166.56, 134.26, 56.31, 55.72, 55.66, 53.83, 52.47, 50.11, 49.98, 39.31, 37.37, 27. 65, 27.21, 23.22.

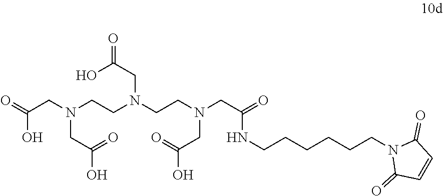 [SMILES: O=C(O)CN(CCN(CC(=O)O)CC(=O)O)CCN(CC(=O)O)CC(=O)NCCCCCCN1C(=O)C=CC1=O]

2-{[2-({2-[bis(carboxymethyl)amino]ethyl}(carboxymethyl)amino)ethyl]({[6-(2,5-dioxo-2,5-dihydro-1H-pyrrol-1-yl)hexyl]carbamoyl}methyl)amino}acetic acid 10d was prepared as described above. Yield: 66 mg (26% referring to the linker) (calculated as TFA salt. TFA content: 1.73±0.25 mol. Eq.), HPLC (method 2, 220 nm) 98%. LRMS-ESI (m/z) calcd. for C₂₄H₃₇N₅O₁₁ [M+H]⁺: 572.58. Found: 572.27. ¹H NMR (400 MHz, D₂O) δ 6.83 (s, 2H), 4.05 (s, 4H), 4.01 (s, 2H), 3.97 (s, 2H), 3.73 (s, 2H), 3.53-3.44 (m, 6H), 3.27-3.21 (m, 6H), 1.59-1.47 (m, 4H), 1.36-1.24 (m, 4H); ¹³C NMR (101 MHz, D₂O, as a TFA salt, the signals of TFA were not included) δ 173.41, 172.92, 169.89, 169.87, 166.34, 134.24, 56.28, 55.59, 55.56, 53.77, 52.58, 50.00, 49.93, 39.41, 37.44, 27.96, 27.43, 25.40, 25.39.

Compounds and metal complexes as shown in Scheme 7 were prepared as described below according to route A or route B.

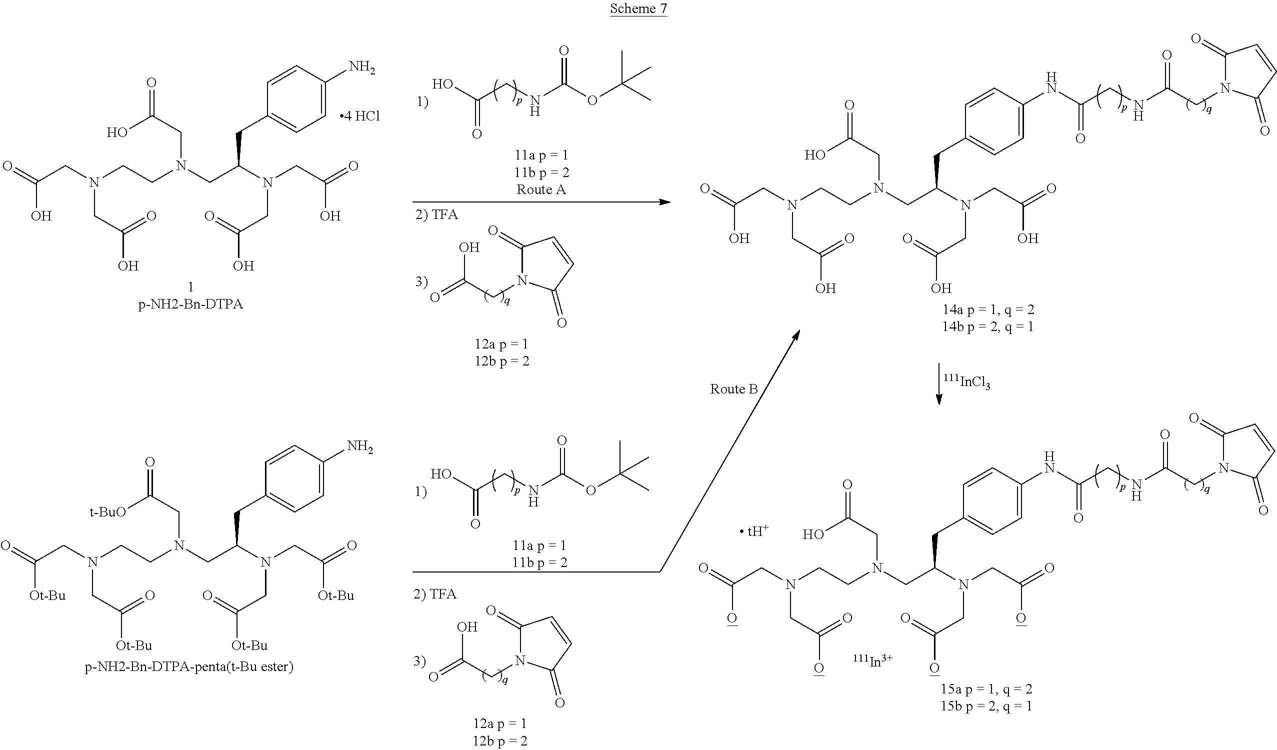

The compounds 17, 18, and 14b were synthesized as described according to route A of Scheme 7 above, as shown and described below in Scheme 8.

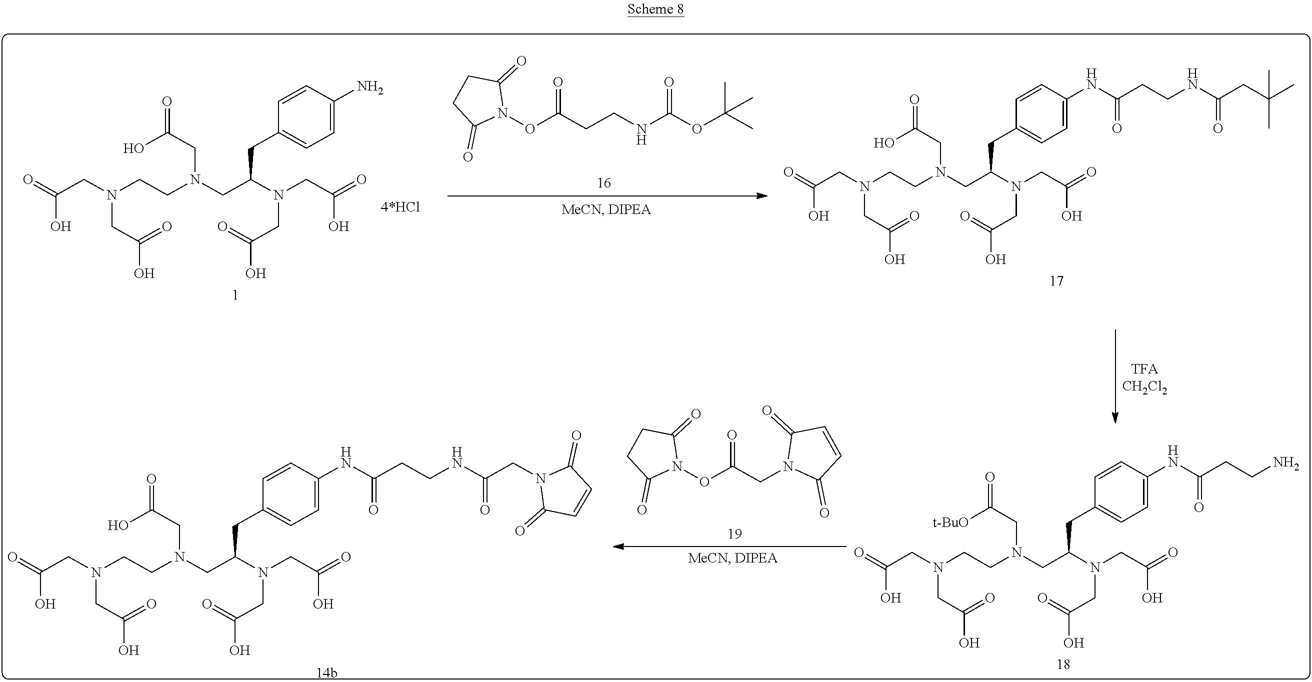

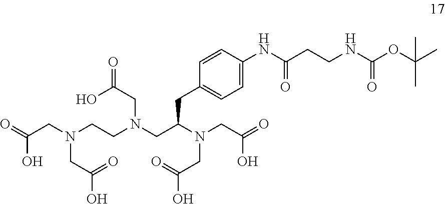 [SMILES: CC(C)(C)OC(=O)NCCC(=O)Nc1ccc(C[C@H](CN(CCN(CC(=O)O)CC(=O)O)CC(=O)O)N(CC(=O)O)CC(=O)O)cc1]

(R)-2,2′-((2-((2-(bis(carboxymethyl)amino)-3-(4-(3-((tert-butoxycarbonyl)amino)propanamido)phenyl)propyl)(carboxymethyl)amino)ethyl)azanediyl)diacetic acid 17 was synthesized as follows. To a solution of/?-NH₂—Bn-DTPA*4 HCl 1 (1.00 eq., 0.46 mmol, 300 mg) in anhydrous MeCN (6 mL) under N₂ atmosphere were added DIEA (11.0 eq., 5.06 mmol, 881 μL) and 2,5-dioxopyrrolidin-1-yl 3-((tert-butoxycarbonyl)amino)propanoate 16 (2.00 eq., 0.93 mmol, 266 mg, as a solution in anhydrous MeCN, 2.00 mL). The reaction mixture was stirred at room temperature, and the conversion of the reaction towards the final product 17 was confirmed by LCMS and HPLC (PDA 220 nm). After ˜18 h, the reaction was quenched by addition of acetic acid (290 μL, 5.06 mmol), and the solvents were partially removed under high vacuum at room temperature. The crude product 17 was re-dissolved in MeCN (2 mL), the solution was cooled to 4° C. and was added to cold MTBE (25 mL). A precipitate was formed and was centrifuged and the supernatant decanted. This process was repeated twice. For FC purification, the resulting precipitate was re-dissolved in water (3 mL) and then purified with a Biotage Isolera One flash purification System with a pre-packed SNAP ULTRA C-18 12 g cartridge, Biotage® HP-Sphere™ C18, 25 μm spherical silica (gradient system from 100% water, 0.1% TFA to 100% MeCN, 0.1% TFA). The product-containing fractions were combined, frozen in liquid nitrogen, and lyophilized for 48 h to afford the target compound 17 as an off-white solid. Yield: 91.0 mg (29%), HPLC (method 6, 220 nm) >95%. LRMS-ESI (m/z) calcd. for C₂₉H₄₃N₅O₁₁ [M+H]⁺: 670.29. Found: 670.25. ¹H NMR (400 MHz, D₂O) δ 7.41-7.39 (m, 2H), 7.31-7.23 (m, 2H), 3.93-3.70 (m, 10H), 3.68-3.57 (m, 1H), 3.44-3.08 (m, 9H), 2.74-2.67 (m, 1H), 2.52 (t, J=6.3 Hz, 2H), 1.33 (s, 9H); ¹³C NMR (101 MHz, D₂O) δ 172.98, 172.53, 172.40, 171.35, 170.50, 135.80, 132.97, 129.81, 122.12, 62.06, 55.70, 55.58, 54.54, 54.09, 52.67, 51.45, 51.35, 49.76, 49.64, 35.47, 31.91, 29.54, 27.54.

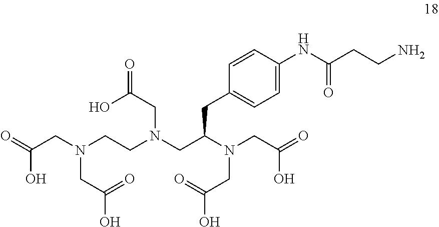 [SMILES: NCCC(=O)Nc1ccc(C[C@H](CN(CCN(CC(=O)O)CC(=O)O)CC(=O)O)N(CC(=O)O)CC(=O)O)cc1]

(R)-2,2′-((2-((3-(4-(3-aminopropanamido)phenyl)-2-(bis(carboxymethyl)amino)propyl)(carboxymethyl)amino)ethyl)azanediyl)diacetic acid 18 was synthesized as follows. To a solution of (R)-2,2′-((2-((2-(bis(carboxymethyl)amino)-3-(4-(3-((/c/7-butoxycarbonyl)amino)propanamido)phenyl)propyl)(carboxymethyl)amino)ethyl) azanediyl)diacetic acid 17 (1.00 eq., 0.13 mmol, 91.0 mg) in anhydrous dichloromethane (2 mL) at 4° C. were added TFA (400 eq., 52.0 mmol, 4 mL) dropwise. The reaction mixture was stirred at room temperature for 4 h. After this time, completion of the reaction was confirmed by LCMS and HPLC (PDA 220 nm). The solvents were removed under high vacuum, and the resulting residue was triturated in a mixture of MeCN, MeOH and MTBE at 4° C. The precipitate was centrifuged, separated from the supernatant and washed twice with cold MTBE (15 mL). The resulting solid was dried under high vacuum for 48 h to afford the target compound 18 as an off-white solid. Yield: 63.2 mg (82%), HPLC (method 7, 220 nm) >91%. LRMS-ESI (m/z) calcd. for C₂₄H₃₅N₅O₁₁ [M+H]⁺: 570.24. Found: 570.16. ¹H NMR (400 MHz, D₂O) δ 7.42 (d, J=8.5 Hz, 2H), 7.28 (d, J=8.5 Hz, 2H), 3.94-3.61 (m, 11H), 3.50-3.35 (m, 2H), 3.31 (t, 7=6.5 Hz, 2H), 3.27-3.03 (m, 5H), 2.83 (t, J=6.6 Hz, 2H), 2.77-2.71 (m, 1H); ¹³C NMR (101 MHz, D₂O) δ 172.08, 171.90, 170.71, 170.17, 135.84, 132.77, 129.88, 122.13, 62.20, 56.12, 54.23, 54.09, 51.83, 49.28, 35.48, 32.50, 31.83.

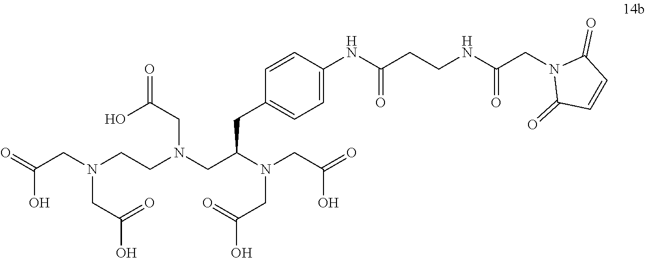 [SMILES: O=C(O)CN(CCN(CC(=O)O)C[C@@H](Cc1ccc(NC(=O)CCNC(=O)CN2C(=O)C=CC2=O)cc1)N(CC(=O)O)CC(=O)O)CC(=O)O]

(R)-2,2′-((2-((2-(bis(carboxymethyl)amino)-3-(4-(3-(2-(2,5-dioxo-2,5-dihydro-1H-pyrrol-1-yl)acetamido)propanamido)phenyl)propyl)(carboxymethyl)amino)ethyl)azanediyl) diacetic acid 14b was synthesized as follows. To a solution of (R)-2,2′-((2-((3-(4-(3-aminopropanamido)phenyl)-2-(bis(carboxymethyl)amino)propyl)(carboxymethyl)amino)ethyl) azanediyl)diacetic acid 18 (1.00 eq., 0.08 mmol, 47.6 mg) in anhydrous MeCN/DMF (2.0:1.5 mL) under N₂ atmosphere were added DIEA (11.0 eq., 0.88 mmol, 153 μL) and maleimidoacetic acid/V-hydroxysuccinimide ester 19 (2.00 eq., 0.17 mmol, 42.0 mg, as a solution in anhydrous MeCN, 2.00 mL). The reaction mixture was stirred at room temperature overnight. After this time, the reaction was quenched by the addition of acetic acid (50 μL, 0.88 mmol), and the solvents were removed under high vacuum at room temperature. The resulting residue was washed twice with MTBE (20 mL), redissolved in water (3 mL) and then purified with a Biotage Isolera One flash purification System with a pre-packed SNAP ULTRA C-18 12 g cartridge, Biotage® HP-Sphere™ C18, 25 μm spherical silica (gradient system from 100% water, 0.1% TFA to 100% MeCN, 0.1% TFA). The product-containing fractions were combined, frozen in liquid nitrogen, and lyophilized for 48 h to afford the target compound 14b as an off-white solid. Yield: 6.0 mg (10%) (calculated as TFA salt. TFA content: 0.47±0.14 mol. eq.). HPLC (method 5, 220 nm) 90%. LRMS-ESI (m/z) calcd. for C₃₀H₃₈N₅O₁₁ [M+H]⁺: 707.25. Found: 707.26. ¹H NMR (400 MHz, D₂O): ¹H NMR (400 MHz, D₂O): δ 7.41 (d, J=8.4 Hz, 2H), 7.31 (d, J=8.4 Hz, 2H), 6.90 (s, 2H), 4.23 (s, 2H), 3.95-3.74 (m, 11H), 3.58 (t, J=6.3 Hz, 2H), 3.52-3.11 (m, 7H), 2.80-2.77 (m, 1H), 2.62 (t, J=6.3 Hz, 2H).

The compounds 20, 21 and 14a in Scheme 9 below were synthesized as described below according to route B of Scheme 7.

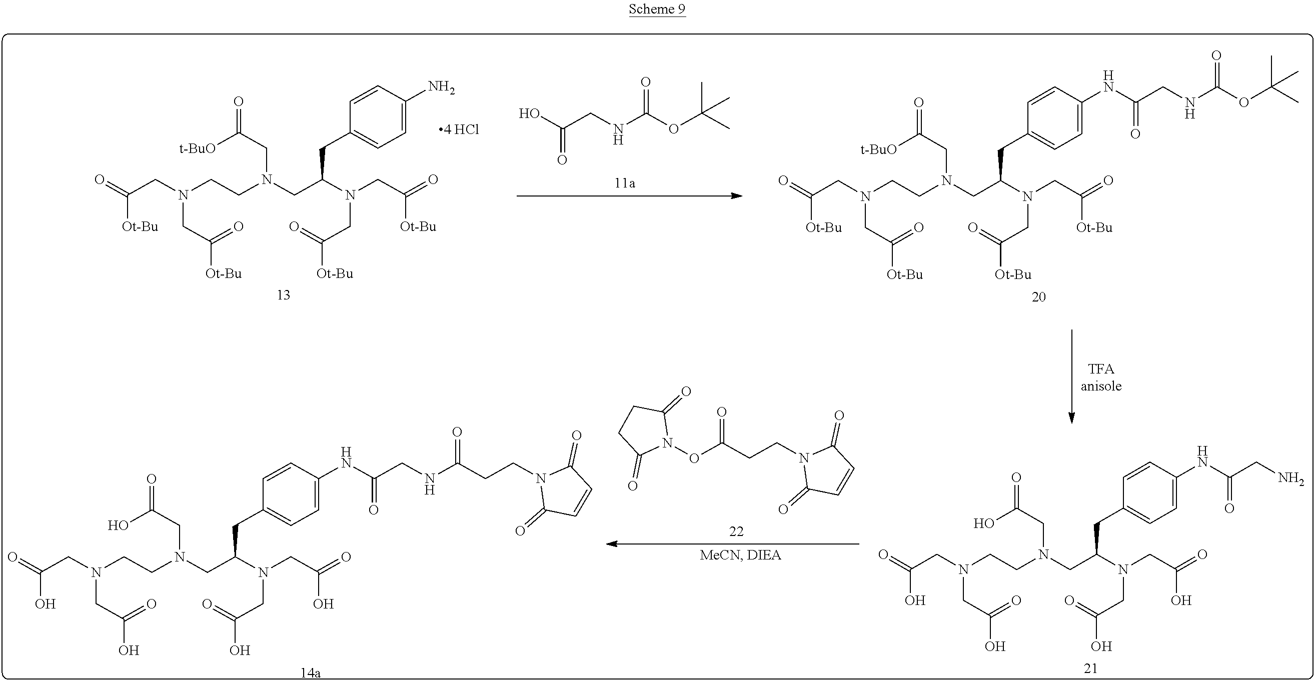

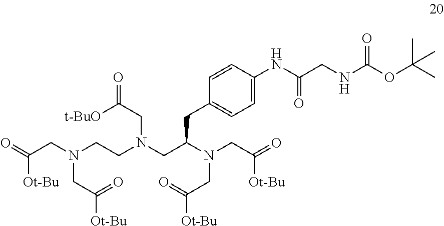 [SMILES: CC(C)(C)OC(=O)CN(CCN(CC(=O)OC(C)(C)C)C[C@@H](Cc1ccc(NC(=O)CNC(=O)OC(C)(C)C)cc1)N(CC(=O)OC(C)(C)C)CC(=O)OC(C)(C)C)CC(=O)OC(C)(C)C]

Di-tert-butyl 2,2′-((2-((2-(bis(2-(tert-butoxy)-2-oxoethyl)amino)-3-(4-(2-((tert-butoxycarbonyl)amino)acetamido)phenyl)propyl)(2-(tert-butoxy)-2-oxoethyl)amino)ethyl)azanediyl)(R)-diacetate 20 was synthesized as follows. HATU (1.30 eq., 0.21 mmol, 79.8 mg) and HOAt (1.30 eq., 0.21 mmol, 28.6 mg) were added to a solution of Boc-Gly-OH 11a (1.30 eq., 0.21 mmol, 36.8 mg) in DMF (3.00 mL). The reaction mixture was stirred at room temperature for 5 min, and then a solution of p-NH₂-Bn-DTPA-penta (7-Bu ester) 13 (1.00 eq., 0.16 mmol, 125 mg) in DMF (2 mL) was added. The reaction mixture was stirred at r.t. for 5 min and 4-methylmorpholine (2.00 eq., 0.32 mmol, 35.0 μL) was added. The mixture was stirred for 4 h at room temperature and then added to a cold saturated solution of NaCl (25 mL). After centrifugation, the precipitate was isolated by decantation, washed with cold water (25 mL) and dried under high vacuum overnight. The crude 20 was purified on a Biotage Isolera One flash purification System, with a pre-packed SNAP ULTRA 25 g cartridge, with Biotage® HP-Sphere™ spherical silica (linear gradient from 100% CHCl₃ to 90/10 CHCl₃/methanol). Solvents were removed first with a rotatory evaporator at 35° C. and then at high vacuum for 48 h to afford the target compound 20 as an off-white solid. Yield: 138 mg (92%), HPLC (method 6, 220 nm) >95%. LRMS-ESI (m/z) calcd. for C₄₈H₈₁N₅O₁₁ [M+H]⁺: 936.59. Found: 936.81.

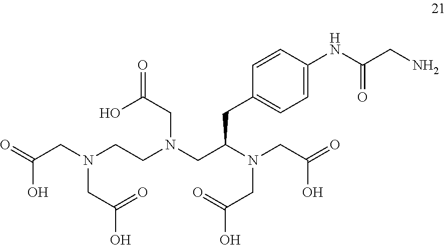 [SMILES: NCC(=O)Nc1ccc(C[C@H](CN(CCN(CC(=O)O)CC(=O)O)CC(=O)O)N(CC(=O)O)CC(=O)O)cc1]

(R)-2,2′-((2-((3-(4-(2-aminoacetamido)phenyl)-2-(bis(carboxymethyl)amino)propyl)(carboxymethyl)amino)ethyl)azanediyl)diacetic acid 21 was synthesized as follows. To a solution of di-tert-butyl 2,2′-((2-((2-(bis(2-(tert-butoxy)-2-oxoethyl)amino)-3-(4-(2-((tert-butoxycarbonyl)amino)acetamido)phenyl)propyl)(2-(/c/7-butoxy)-2-oxoethyl)amino)ethyl)azanediyl)(R)-diacetate (1.00 eq., 0.15 mmol, 138 mg) in anhydrous anisole (3.00 mL) at 4° C. was added TFA (350 eq., 52.2 mmol, 4.00 mL) dropwise. The reaction mixture was stirred at room temperature for 24 h. After this time, completion of the reaction was confirmed by LC-MS. Solvents were removed under high vacuum and the product was triturated in a mixture of MeCN, MeOH and MTBE at 4° C. The resulting precipitate was centrifuged, separated from the supernatant and washed twice with cold MTBE (15 mL). The resulting solid was redissolved in water (5 mL) and then purified using a Biotage Isolera One flash purification System equipped with a pre-packed SNAP ULTRA C-18 30 g cartridge, Biotage® HP-Sphere™ C18, 25 μm spherical silica (gradient system from 100% water, 0.1% TFA to 85% water, 0.1% TFA: 15% MeCN, 0.1% TFA). The product-containing fractions were combined, frozen in liquid nitrogen, and lyophilized for 48 h to afford the title compound 21 as an off-white solid. Yield: 65.8 mg (80%), HPLC (method 5, 220 nm) >95%. LRMS-ESI (m/z) calcd. for C₂₃H₃₃N₅O₁₁ [M+H]+: 556.23. Found: 556.22. ¹H NMR (400 MHz, D₂O) δ 7.45 (d, J=8.3 Hz, 2H), 7.29 (d, J=8.3 Hz, 2H), 4.00 (s, 2H), 3.92-3.70 (m, 10H), 3.69-3.58 (m, 1H), 3.49-3.05 (m, 7H), 2.72 (dd, J=13.8, 9.3 Hz, 1H); ¹³C NMR (101 MHz, D₂O) δ 172.34, 171.58, 170.43, 165.38, 135.52, 133.08, 129.98, 121.75, 62.07, 55.82, 54.42, 54.13, 52.84, 51.52, 49.64, 40.94, 31.93.

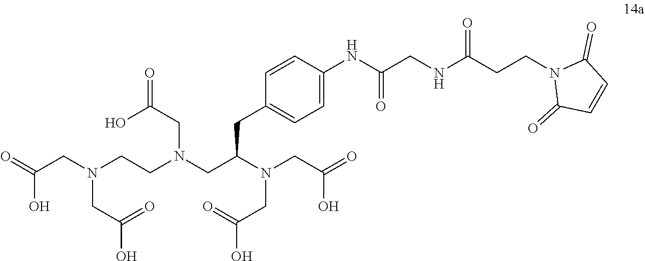 [SMILES: O=C(O)CN(CCN(CC(=O)O)C[C@@H](Cc1ccc(NC(=O)CNC(=O)CCN2C(=O)C=CC2=O)cc1)N(CC(=O)O)CC(=O)O)CC(=O)O]

(R)-2,2′-((2-((2-(bis(carboxymethyl)amino)-3-(4-(2-(3-(2,5-dioxo-2,5-dihydro-1H-pyrrol-1-yl)propanamido)acetamido)phenyl)propyl)(carboxymethyl)amino)ethyl)azanediyl) diacetic acid 14a was synthesized as follows. To a solution of (R)-2,2′-((2-((3-(4-(2-aminoacetamido)phenyl)-2-(bis(carboxymethyl)amino)propyl)(carboxymethyl)amino)ethyl) azanediyl)diacetic acid (1.00 eq, 0.11 mmol, 60.0 mg) in anhydrous MeCN:DMF (2.00:1.00 mL) under N₂ atmosphere were added DIEA (11.0 eq, 1.21 mmol, 211 μL) and N-succinimidyl-3-maleimidopropionate 22 (2.00 eq., 0.22 mmol, 57.5 mg, as a solution in anhydrous MeCN:DMF, 2.00:0.50 mL). The reaction mixture was stirred at room temperature overnight. After this time, the reaction was quenched by the addition of acetic acid (1.21 mmol, 69.0 μL) and solvents were removed under high vacuum at room temperature. The resulting residue was washed twice with 20.0 mL MTBE. The resulting precipitate was redissolved in water (3.00 mL) and then purified with a Biotage Isolera One flash purification System with a pre-packed SNAP ULTRA C-18 12 g cartridge, Biotage® HP-Sphere™ C18, 25 μm spherical silica (gradient system from 100% water/0.1% TFA to 100% MeCN/0.1% TFA). The product-containing fractions were combined, frozen in liquid nitrogen, and lyophilized for 48 h to afford the target compound 14a as an off-white solid. Yield: 18.0 mg (24%) (calculated as TFA salt. TFA content: 0.63±0.14 mol. eq.) HPLC (220 nm, method 5) >96%. LRMS-ESI (m/z) calcd. for C₃₀H₃₈N₆O₁₄ [M+H]⁺: 707.25. Found: 707.28. ¹H NMR and ¹³C NMR: ¹H NMR (400 MHz, D₂O): ¹H NMR (400 MHz, D₂O) δ 7.41 (d, J=8.5 Hz, 2H), 7.30 (d, J=8.5 Hz, 2H), 6.82 (s, 2H), 3.99 (s, 2H), 3.94-3.88 (m, 6H), 3.87-3.76 (m, 6H), 3.69-3.58 (m, 1H), 3.47-3.11 (m, 7H), 2.81-2.71 (m, 1H), 2.63 (t, J=8.0 Hz, 2H); ¹³C NMR (101 MHz, D₂O) δ 174.23, 172.65, 172.61, 171.23, 170.59, 169.76, 135.59, 134.39, 133.17, 129.88, 122.49, 62.05, 55.48, 54.50, 54.09, 52.71, 51.26, 49.87, 43.08, 34.19, 31.92.

### Example 3

### Serum Albumin Binding of ¹¹¹In-C4-DTPA and Complex Stability

The radiolabeling of C4-DTPA was performed by adding 57.7±0.7 MBq (C4-DTPA) of ¹¹¹InCl₃ in HCl solution (MAP Medical, Finland) into vials containing lyophilized test compound. The vials were vortexed until a clear solution was achieved and incubated for 30 minutes at +37° C.

The radiochemical purity of the radiolabeled product ¹¹¹In-C4-DTPA was measured with reverse phase Aeris Widepore C18 column (Agilent 1260 Infinity II chromatography system, HPLC method 9). The sample was diluted 1:30 (V:V) with water and 5 μl were injected into the Radio-HPLC system. Binding studies with the ¹¹¹In-C4-DTPA complex in serum: NMRI murine serum and human serum were removed from −80° C. storage and allowed to reach room temperature. The thawed serums were spun down at 13.6 kRPM for 60 seconds, the supernatant was filtered through a filter needle (5 μm) and subsequently through a 0.45 μm CA membrane.

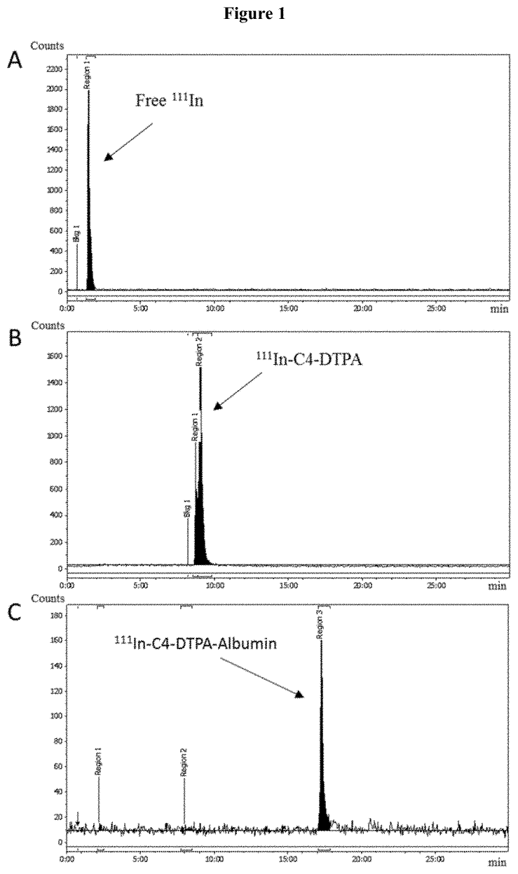

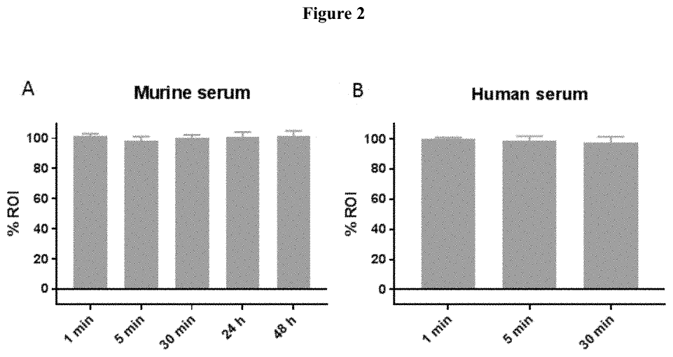

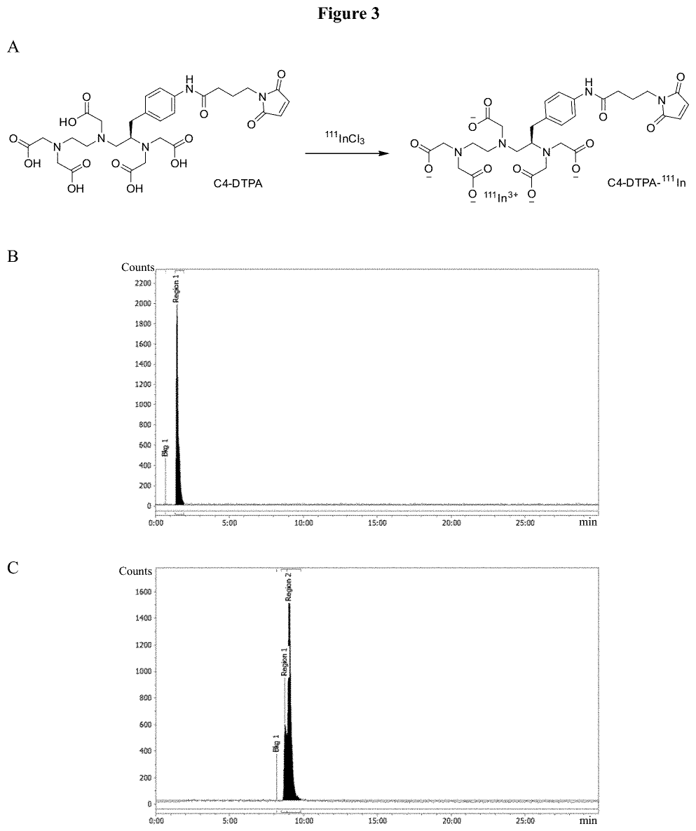

The serum was incubated at 37° C. for 30 minutes prior to addition of ¹¹¹In-C4-DTPA. After 30 minutes pre-incubation of the serum, the ¹¹¹In-C4-DTPA solution was added to the serum in 1:30 (V:V) ratio. Samples were taken after 1 minute, 5 minutes and 30 minutes and analyzed by Radio-HPLC, 5 μl were injected for each time point. All binding experiments were carried out in triplicate (n=3). The stability of the ¹¹¹In-C4-DTPA-albumin conjugate in the mouse serum was in addition monitored over 48 hours at 37° C. The concentration of free indium, unbound ¹¹¹In-C4-DTPA and ¹¹¹In-C4-DTPA albumin conjugate were determined in each measurement as demonstrated by HPLC radiographs in FIG. 1. Albumin binding was determined by comparing AUC for each measurement to that of ¹¹¹In-C4-DTPA in water, rate of binding and complex stability is presented in FIG. 2.

### Example 4

### Pharmacokinetic Study in Naïve NMRI Mice

Radiolabeling: The radiolabeling of C4-DTPA was performed as described above with the exception of using 220 MBq of ¹¹¹In in the radiolabeling and 1:40 dilution with water for quality control sample.

Dosing: Total of 20 NMRI mice were dosed intravenously with ¹¹¹In-C4-DTPA (6.2±0.1 MBq). The total injection volume was 100 μl which was prepared by mixing 35 μl of radiolabeled metal complex and 65 μl of sterile saline (B. Braun).

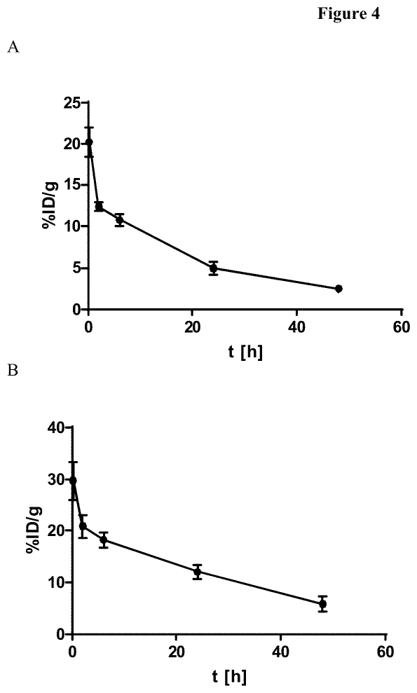

End-point sampling: Samples were collected 10 min, 1 h, 6 h, 24 h and 48 h after intravenous dosing (n=4). At the specified time point the mice were terminated with overdose of CO₂ and opening of chest cavity. A blood sample was drawn via cardiac puncture. Small volume of blood was taken into separate tube and the remaining blood sample was stored on ice until plasma separation. Plasma was separated by centrifugation, 2000 G for 10 minutes at +4° C. Radioactivity was measured with a gamma counter (Wizard II, Perkin Elmer). Data was presented in FIG. 4 as % of injected dose/g of tissue (% ID/g).

### Example 5

### In Vivo SPECT/CT Imaging

Tumor implantation: Female immunodeficient NMRI nude mice were provided by Charles River, Freiburg, Germany. Animals received bilateral subcutaneous tumor implants with PDX model LXFL 529 (NSCLC) or OVXF 899 (ovarian cancer) in the flanks while under isoflurane anesthesia. Animals were kept in cages, the temperature inside the cages was maintained at 25±1° C. with a relative humidity of 45-65% and an air change rate in the cage of 60-fold per hour. They were kept under a 14-hour light/10-hour dark, artificial light cycle. Food and water were provided ad libitum. The imaging studies started when the individual tumors were palpable and had reached a volume of 100-400 mm³. The body weight and tumor size of s.c. PDX tumor bearing NMRI mice was measured daily (n=8 per tumor model). Tumor diameter was measured with calipers in two axis and tumor volume is calculated with formulaV=(L×W×W)/2, where V is tumor volume, W is tumor width, L is tumor length.

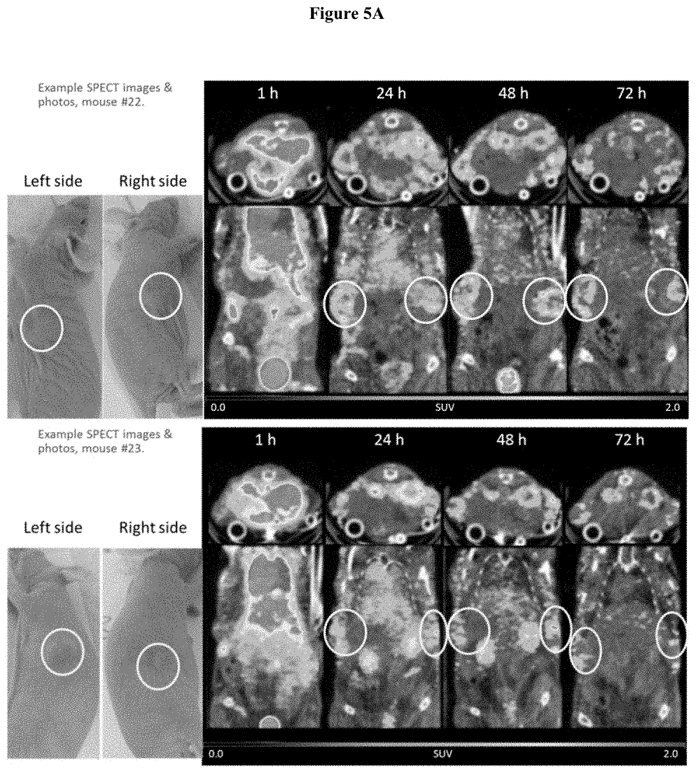

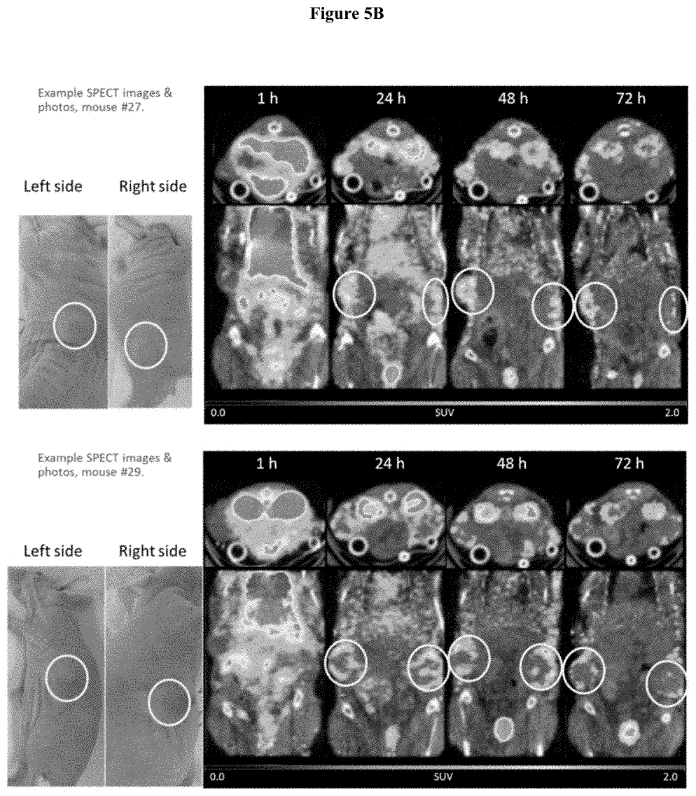

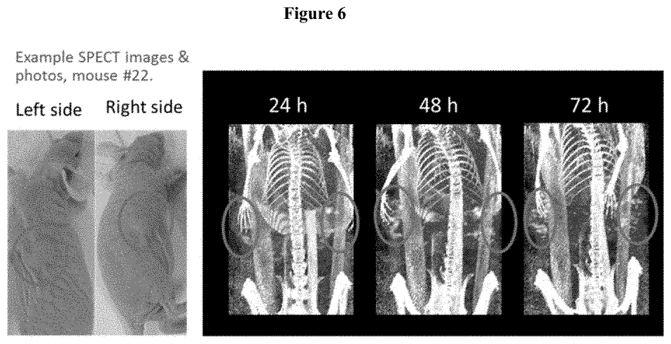

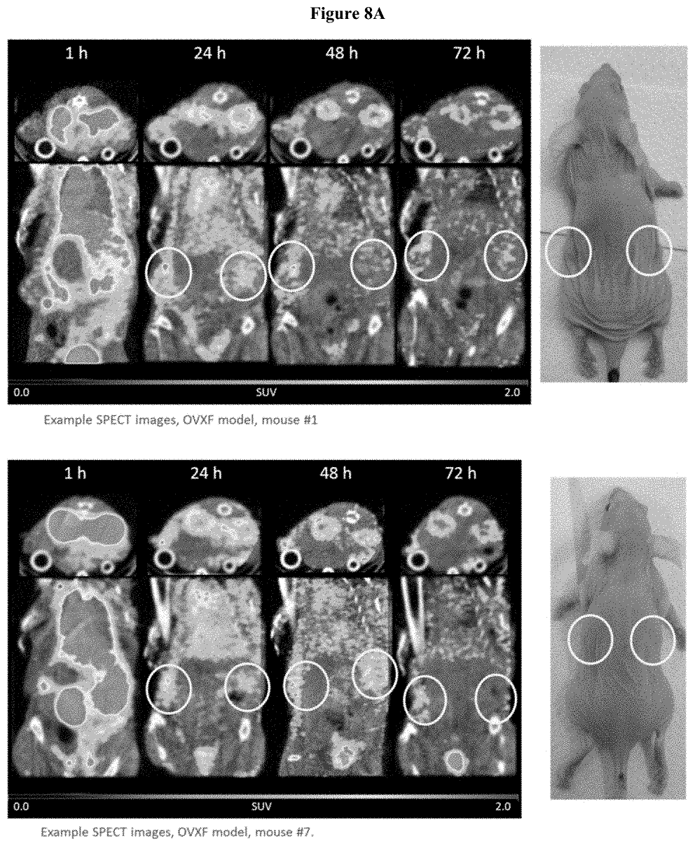

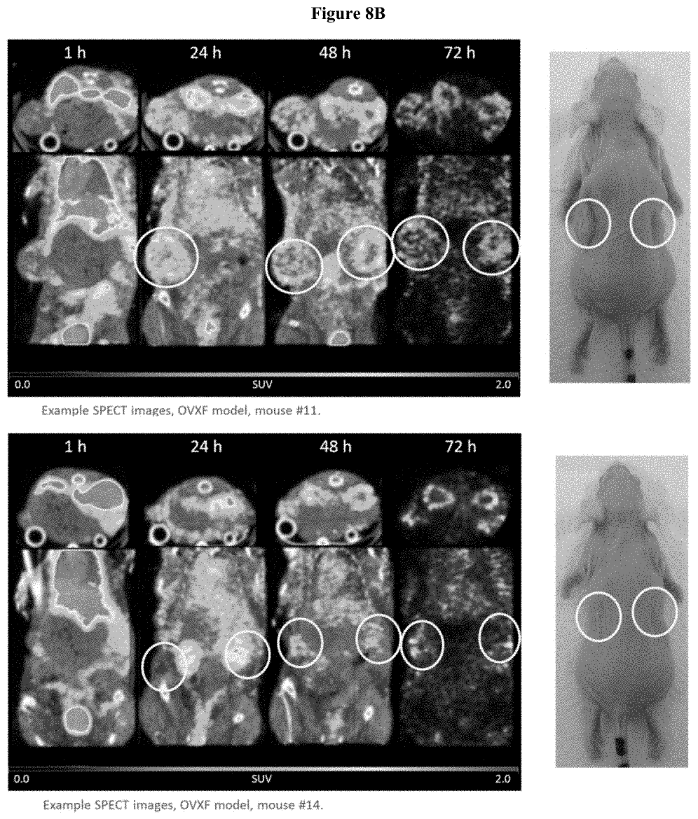

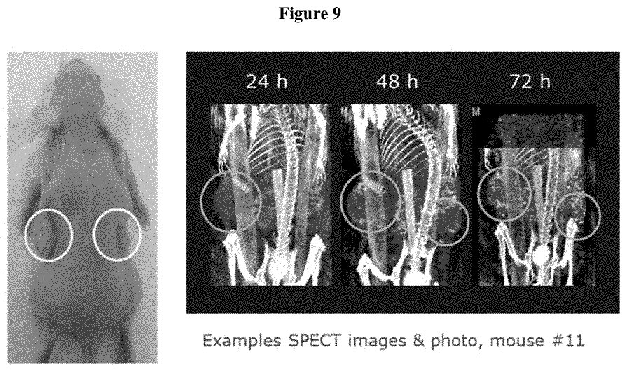

SPECT/CT Imaging: After animals have reached average tumor volume between 100 mm³-300 mm³, the mice were anesthetized and subjected to i.v. injection of ¹¹¹In-C4-DTPA (ca. 20 MBq). SPECT/CT imaging was performed with a small animal SPECT/CT (NanoSPECT/CT Plus, Mediso) at 0-1 h, 24 h, 48 h and 72 h post-dosing. 3D images of the animals combined with CT were produced to visualize biodistribution of labeled agent. Imaging protocol consists of planar tomography image which was used as a reference to choose imaging area (tumors are in the center of field of view). After choosing the imaging area, helical CT was performed (180 projections, 55 kVp, 750 ms exposure time). Finally, helical SPECT scan was performed from the same coordinates using 90 s/time frame. High resolution multipin-hole apertures were used to enhance resolution. After SPECT imaging Hi SPECT reconstruction was used for the SPECT images. Image analysis was performed using PMOD software v3.7. See FIGS. 5A, 5B, 6, 8A, 8B and 9.

End-point sampling: After the 72 h imaging time point the mice were terminated with an overdose of CO₂ and opening of the chest cavity. A blood sample was drawn via cardiac puncture. Small volumes of blood were collected into separate tubes and the remaining blood samples stored on ice until plasma separation. Plasma was separated by centrifugation, 2000 G for 10 minutes at +4° C.

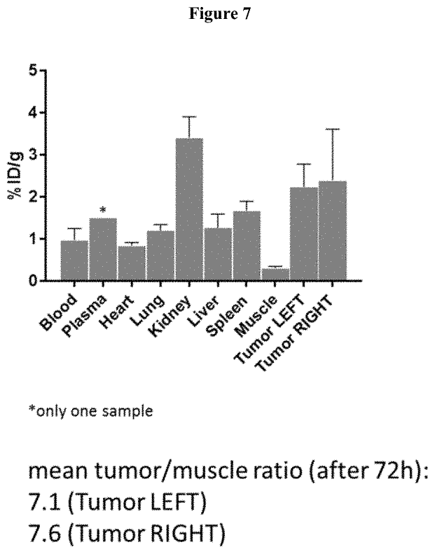

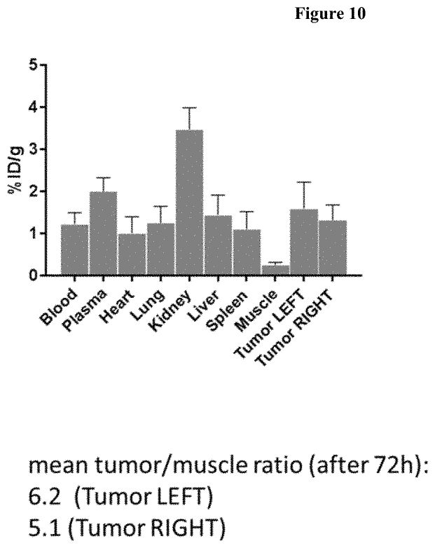

Samples from lungs, heart, liver, spleen, kidneys, tumors and muscle (femoris) were collected into pre-weighed tubes. After sample collection the tubes were weighed again and the radioactivity was measured with a gamma counter (Wizard II, Perkin Elmer). Data is presented as % of injected dose/g of tissue (% ID/g). See FIGS. 7 and 10.

Particular embodiments of the invention are set forth in the following numbered paragraphs:

1. A metal complex having the structure of Formula (I) or (II) or (III):

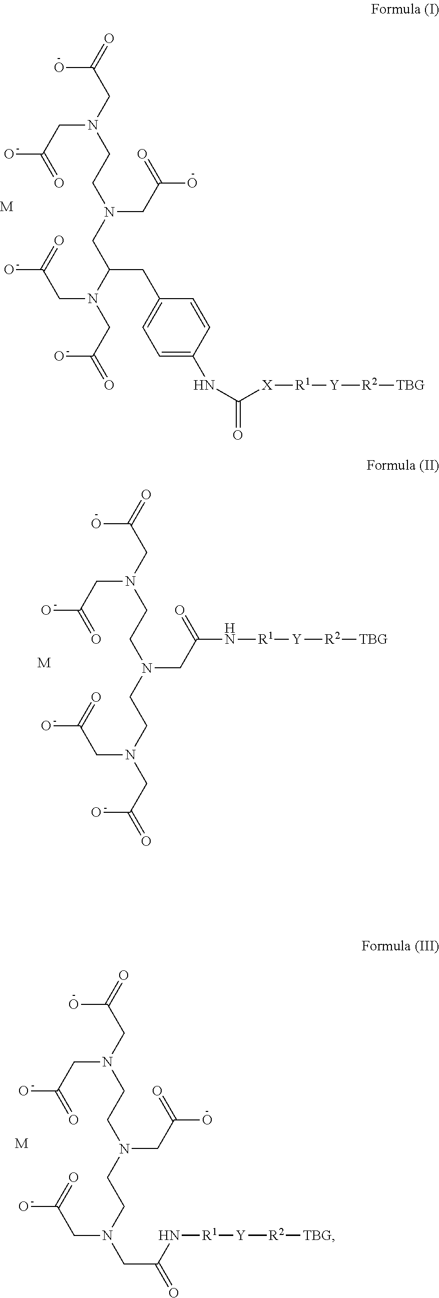

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
X is absent or selected from —NH—, and —O—;
R¹ is absent or an optionally substituted C₁—C ix alkyl wherein optionally up to six carbon atoms in said C₁-C₁₈ alkyl are each independently replaced with —OCH₂CH₂—;
Y is absent or selected from —O—C(O)—, —C(O)—O—, —NH—C(O)—, —C(O)—NH—, —NH—C(O)—NH—, —O—C(O)—O—, —NH—C(O)—O—, and —O—C(O)—NH—;
R² is an optionally substituted C₁-C₁₈ alkyl wherein optionally up to six carbon atoms in said C₁-C₁₈ alkyl are each independently replaced with —OCH₂CH₂—; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.
   2. The metal complex of paragraph 1, having the structure of Formula (I):

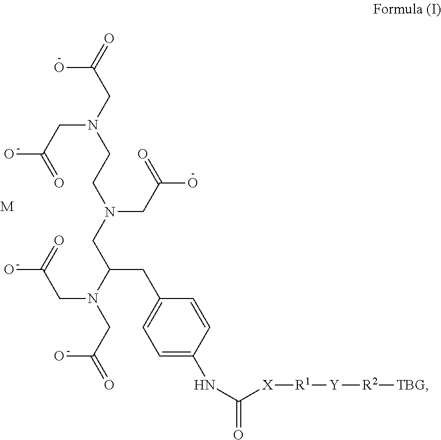

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
X is absent or selected from —NH—, and —O—;
R¹ is absent or an optionally substituted C₁-C₁₈ alkyl wherein optionally up to six carbon atoms in said C₁-C₁₈ alkyl are each independently replaced with —OCH₂CH₂—;
Y is absent or selected from —O—C(O)—, —C(O)—O—, —NH—C(O)—, —C(O)—NH—, —NH—C(O)—NH—, —O—C(O)—O—, —NH—C(O)—O—, and —O—C(O)—NH—;
R² is an optionally substituted C₁-C₁₈ alkyl wherein optionally up to six carbon atoms in said C₁-C₁₈ alkyl are each independently replaced with —OCH₂CH₂—; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.
   3. The metal complex of paragraph 1, having the structure of Formula (II):

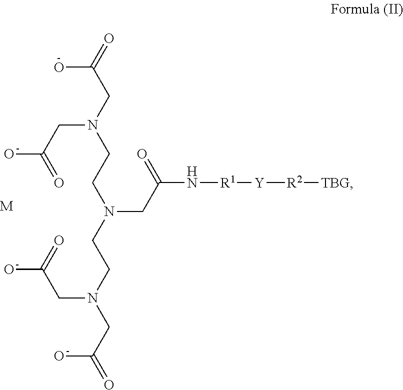

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
R¹ is absent or an optionally substituted C₁-C₁₈ alkyl wherein optionally up to six carbon atoms in said C₁-C₁₈ alkyl are each independently replaced with —OCH₂CH₂—;
Y is absent or selected from —O—C(O)—, —C(O)—O—, —NH—C(O)—, —C(O)—NH—, —NH—C(O)—NH—, —O—C(O)—O—, —NH—C(O)—O—, and —O—C(O)—NH—;
R² is an optionally substituted C₁-C₁₈ alkyl wherein optionally up to six carbon atoms in said C₁-C₁₈ alkyl are each independently replaced with —OCH₂CH₂—; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.
   4. A metal complex of paragraph 1, having the structure of Formula (III):

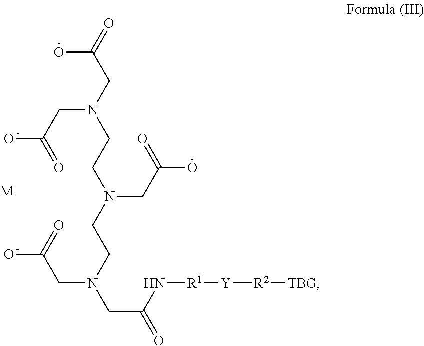

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
R¹ is absent or an optionally substituted C₁-C₁₈ alkyl wherein optionally up to six carbon atoms in said C₁-C₁₈ alkyl are each independently replaced with —OCH₂CH₂—;
Y is absent or selected from —O—C(O)—, —C(O)—O—, —NH—C(O)—, —C(O)—NH—, —NH—C(O)—NH—, —O—C(O)—O—, —NH—C(O)—O—, and —O—C(O)—NH—;
R² is an optionally substituted C₁-C₁₈ alkyl wherein optionally up to six carbon atoms in said C₁-C₁₈ alkyl are each independently replaced with —OCH₂CH₂—; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.
   5. The metal complex according to paragraph 1 having the structure of Formula (IV), (V) or (VI):

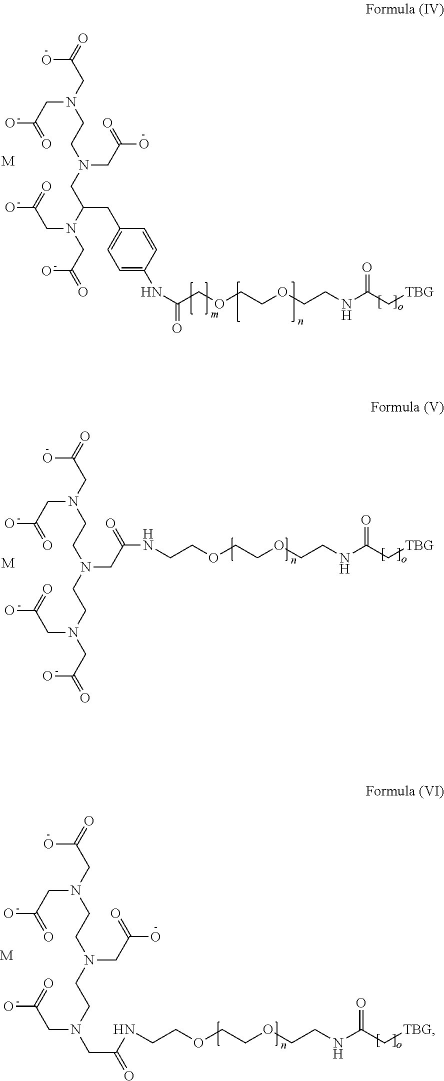

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
m=1 or 2;
n=1-5;
o=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.
   6. The metal complex according to paragraph 5 having the structure of Formula (IV):

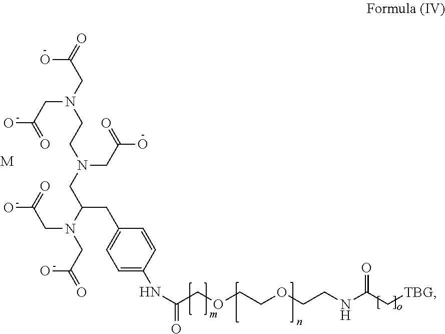

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
m=1 or 2;
n=1-5;
o=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.
   7. The metal complex according to paragraph 5 having the structure of Formula (V):

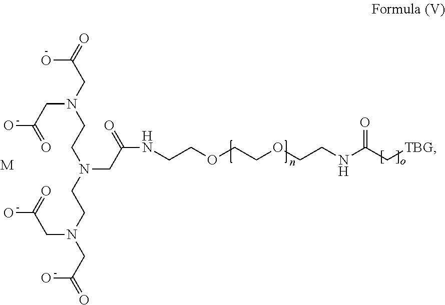

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
n=1-5;
o=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.
   8. The metal complex according to paragraph 5 having the structure of Formula (VI):

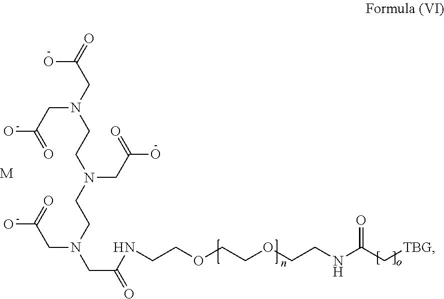

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
n=1-5;
o=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.
   9. The metal complex according to paragraph 1 having the structure of Formula (VII), (VIII) or (IX):

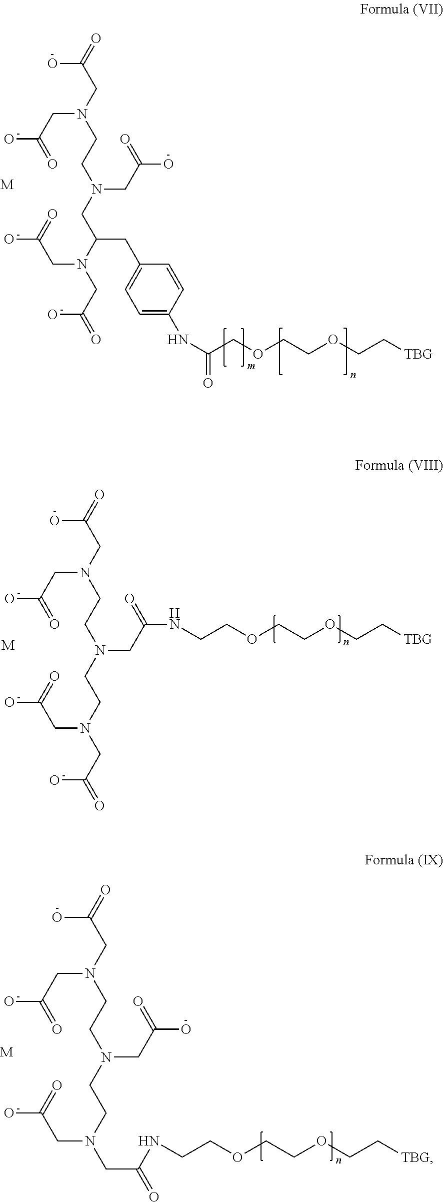

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
m=1 or 2;
n=1-5; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.
   10. The metal complex according to paragraph 9 having the structure of Formula (VII):

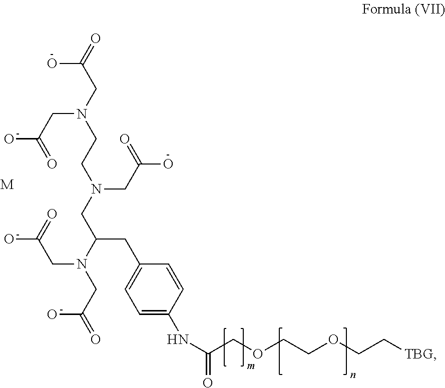

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
m=1 or 2;
n=1-5; and TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.
   11. The metal complex according to paragraph 9 having the structure of Formula (VIII):

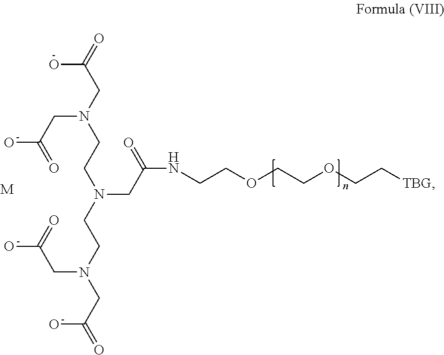

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
n=1-5; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.
   12. The metal complex according to paragraph 9 having the structure of Formula (IX):

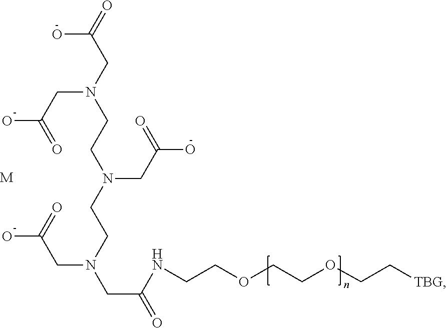

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
n=1-5; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.
   13. The metal complex according to paragraph 1 having the structure of Formula (X), (XI) or (XII):

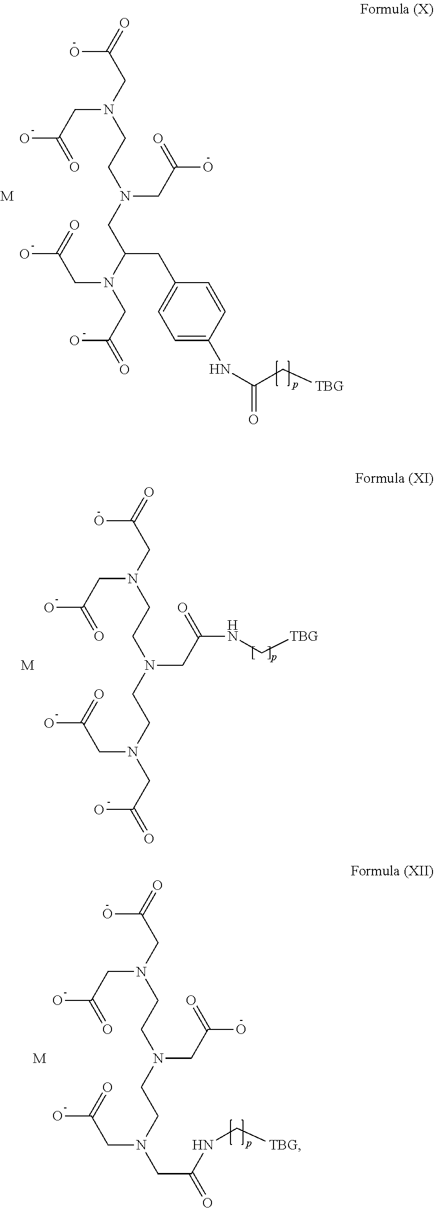

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
p=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.
   14. The metal complex according to paragraph 13 having the structure of Formula (X):

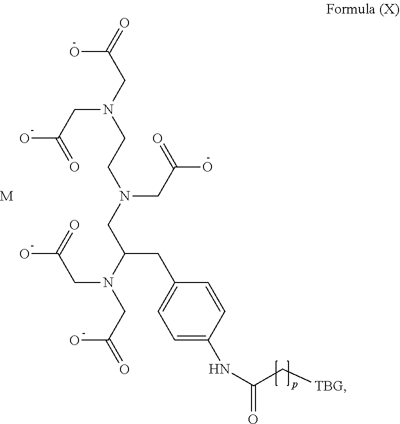

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
p=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene.
   15. The metal complex according to paragraph 13 having the structure of Formula (XI):

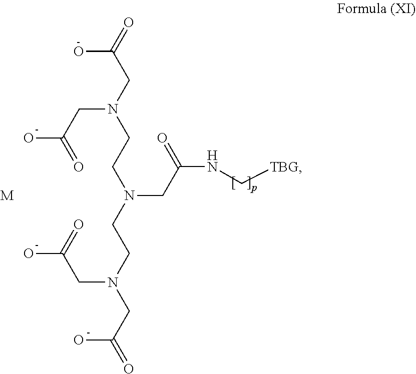

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
p=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.
   16. The metal complex according to paragraph 13 having the structure of Formula (XII):

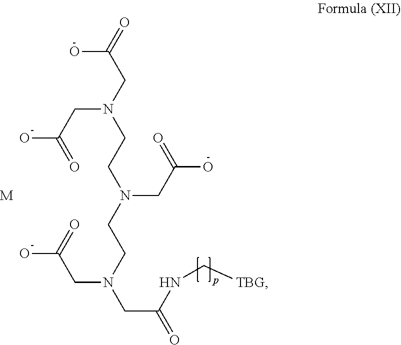

or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
p=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.
   17. The metal complex according to any one of paragraphs 1-16, a conjugate acid thereof, or a pharmaceutically acceptable salt or hydrate thereof, wherein TBG is an optionally substituted maleimide group.
   18. The metal complex according to any one of paragraphs 1-17, a conjugate acid thereof, or a pharmaceutically acceptable salt or hydrate thereof, wherein TBG is a maleimide group,

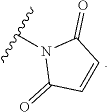 [SMILES: *N1C(=O)C=CC1=O]

19. The metal complex according to any one of paragraphs 1-18, a conjugate acid thereof, or a pharmaceutically acceptable salt or hydrate thereof, wherein M is ¹¹¹In³⁺.
20. The metal complex according to paragraph 1, wherein the metal complex is selected from:

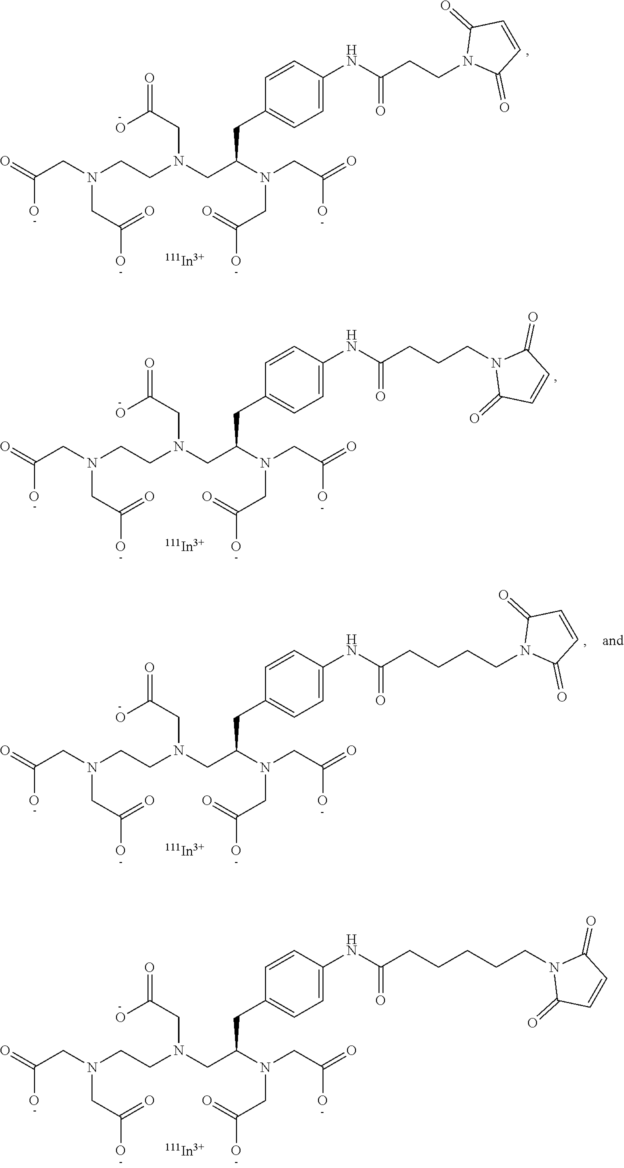

or a conjugate acid thereof, or a pharmaceutically acceptable salt or hydrate thereof.
21. The metal complex according to paragraph 1, wherein the metal complex is selected from:

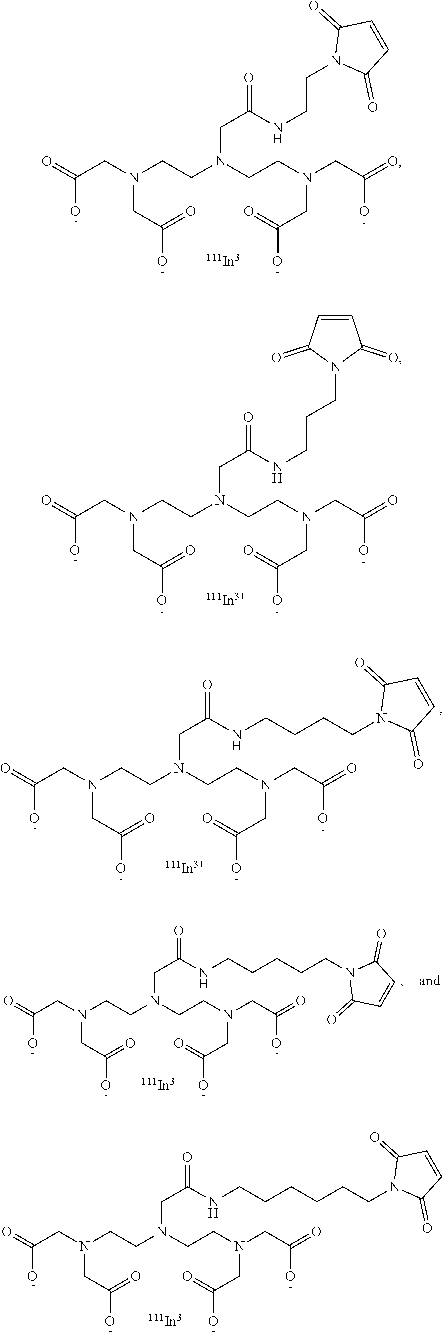

or a conjugate acid thereof, or a pharmaceutically acceptable salt or hydrate thereof.
22. The metal complex according to paragraph 1, wherein the metal complex is selected from:

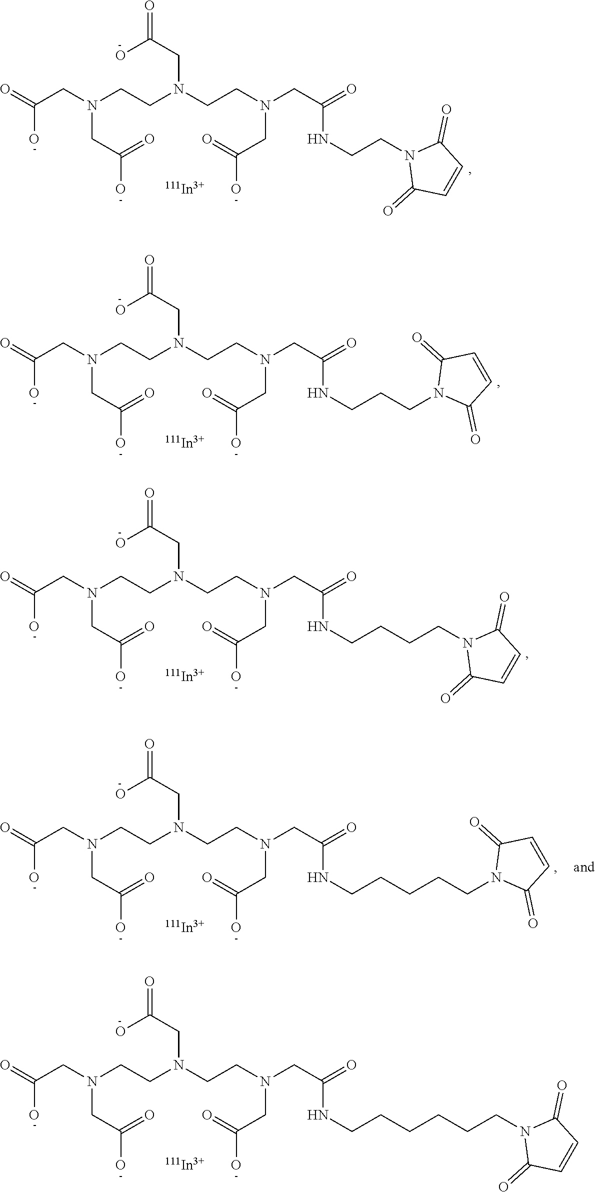

or a conjugate acid thereof, or a pharmaceutically acceptable salt or hydrate thereof.
23. The metal complex according to paragraph 1, wherein the metal complex is selected

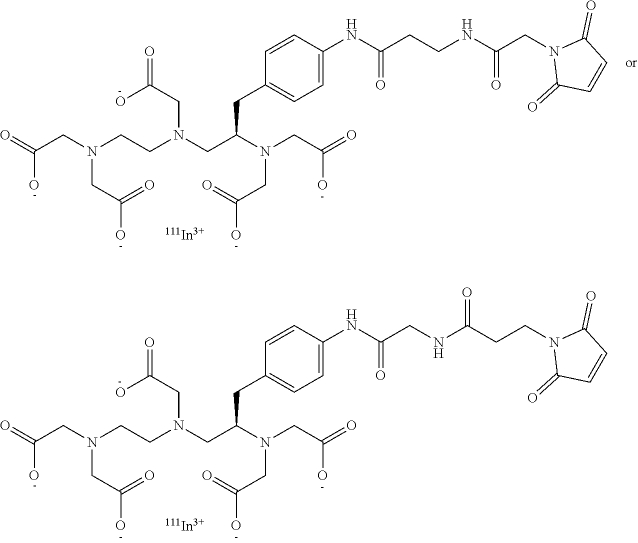

or a conjugate acid thereof, or a pharmaceutically acceptable salt or hydrate thereof.
24. The metal complex according to any one of paragraphs 1-23, wherein the counter cation of the pharmaceutically acceptable salt is selected from: one or two Na⁺, K⁺, or NH₄⁺; or one Ca²⁺ or Mg²⁺.
25. A pharmaceutical composition comprising the metal complex of any of one of paragraphs 1-24, a conjugate acid thereof, or a pharmaceutically acceptable salt or hydrate thereof, optionally comprising one or more pharmaceutical acceptable carrier.
26. The pharmaceutical composition according to paragraph 25, wherein the metal complex covalently binds to the thiol group of cysteine-34 of endogenous or exogenous albumin.
27. A method for diagnosing a disease selected from a cancer, a viral disease, autoimmune disease, acute or chronic inflammatory disease, and a disease caused by bacteria, fungi, or other micro-organisms, comprising administering to a subject in need thereof a diagnostically effective amount of a metal complex according to any of paragraphs 1-24 or a pharmaceutical composition according to paragraph 25 or 26, and subsequent SPECT imaging (single-photon emission computed tomography).
28. The method of paragraph 27, wherein the disease is cancer.
29. A method of diagnosing cancer in a subject, the method comprising:

administering a detectable amount of the metal complex of any one of paragraphs 1-24 or a pharmaceutical composition of paragraph 25 or 26, to the subject;

imaging the subject after administering the metal complex or pharmaceutical composition to detect a signal from a radiolabel of the metal complex, wherein a presence of the radiolabel in a tissue indicates that the tissue is cancerous, thereby diagnosing cancer in the subject.

30. A method of diagnosing cancer in a subject, the method comprising:

administering a detectable amount of the metal complex of any one of paragraphs 1-24 or a pharmaceutical composition of paragraph 25 or 26, to the subject;

imaging the subject after administering the metal complex or pharmaceutical composition, to detect a signal from a radiolabel of the metal complex, wherein a presence of a higher accumulation of the signal in a tissue in comparison to noncancerous tissue of the same type indicates that the tissue is cancerous, thereby diagnosing cancer in the subject.

31. A method of diagnosing cancer in a subject, the method comprising:

administering to the subject the metal complex of any one of paragraphs 1-24 or a pharmaceutical composition of paragraph 25 or 26, wherein the metal complex binds to albumin to form a metal complex-albumin conjugate, wherein the metal complex-albumin conjugate accumulates in cancerous tissue;

imaging the subject after administering the metal complex or pharmaceutical composition, to detect a signal from a radiolabel of the metal complex, wherein a presence of the radiolabel in the tissue indicates that the tissue is cancerous, thereby diagnosing cancer in the subject.

32. A method of treating cancer in a subject, the method comprising:

administering to the subject the metal complex of any one of paragraphs 1-24 or a pharmaceutical composition of paragraph 25 or 26, wherein the metal complex binds to albumin forming a metal complex-albumin conjugate, wherein the metal complex-albumin conjugate accumulates in cancerous tissue;

imaging the subject after administering the metal complex or pharmaceutical composition, to detect a signal from a radiolabel of the metal complex, wherein a presence of the radiolabel in the tissue indicates that the tissue is cancerous;

diagnosing the subject with cancer in the tissue; and administering a therapeutically effective amount of a chemotherapeutic agent to the subject.

33. A method of diagnosing and treating a subject with a cancer responsive to an albumin-binding chemotherapeutic agent, the method comprising:

administering to the subject the metal complex of any one of paragraphs 1-24 or a pharmaceutical composition of paragraph 25 or 26, wherein the metal complex binds to albumin forming a metal complex-albumin conjugate, wherein the metal complex-albumin conjugate accumulates in cancerous tissue;

imaging the subject after administering the metal complex or pharmaceutical composition, to detect a signal from a radiolabel of the metal complex, wherein a presence of the radiolabel in the tissue indicates that the tissue is cancerous;

diagnosing the subject with cancer that is responsive to treatment with the albumin-binding chemotherapeutic agent; and

administering a therapeutically effective amount of the albumin-binding chemotherapeutic agent to the subject.

34. A method comprising:

administering to a subject the metal complex of any one of paragraphs 1-24 or a pharmaceutical composition of paragraph 25 or 26, wherein the metal complex binds to albumin forming a metal complex-albumin conjugate, wherein the metal complex-albumin conjugate accumulates in cancerous tissue;

imaging the subject after administering the metal complex or pharmaceutical composition, to detect a signal from a radiolabel of the metal complex, wherein a presence of the radiolabel in the tissue indicates that the tissue is cancerous;

diagnosing the subject with cancer in the tissue; and administering a therapeutically effective amount of an albumin-binding chemotherapeutic agent to the subject.

35. A method comprising:

administering to a subject the metal complex of any one of paragraphs 1-24 or a pharmaceutical composition of paragraph 25 or 26, wherein the metal complex binds to albumin forming a metal complex-albumin conjugate, wherein the metal complex-albumin conjugate accumulates in cancerous tissue;

imaging the subject after administering the metal complex or pharmaceutical composition, to detect a signal from a radiolabel of the metal complex, wherein a presence of the radiolabel in the tissue indicates that the tissue is cancerous;

diagnosing the subject with cancer in the tissue;

classifying the subject as being responsive to an albumin-binding chemotherapeutic agent; and

administering a therapeutically effective amount of the albumin-binding chemotherapeutic agent to the subject.

36. A method for assessing the responsiveness of a cancer in a subject to an albumin-binding chemotherapeutic agent comprising:

administering to a subject the metal complex of any one of paragraphs 1-24 or a pharmaceutical composition of paragraph 25 or 26, wherein the metal complex binds to albumin to form a metal complex-albumin conjugate, wherein the metal complex-albumin conjugate accumulates in cancerous tissue;

imaging the subject after administering the metal complex or pharmaceutical composition, to detect a signal from a radiolabel of the metal complex, wherein a presence of the radiolabel in the tissue indicates that the tissue is cancerous;

diagnosing the subject with cancer in the tissue; and

classifying the subject as having a cancer responsive to the albumin-binding chemotherapeutic agent.

37. The method of any one of paragraphs 27-36, wherein the metal complex is administered as a metal complex-albumin conjugate formed ex vivo.
38. The method of paragraph 37, wherein metal complex-albumin conjugate is formed by conjugation of albumin to a moiety corresponding to the TBG of the metal complex; followed by chelation of M.
39. The method of paragraph 37, wherein metal complex-albumin conjugate is formed by chelation of M to form the metal complex; followed by conjugation of albumin to the TBG of the metal complex to form the metal complex-albumin conjugate.
40. The method of any one of paragraphs 27-38, wherein the cancer is selected from adenocarcinoma, uveal melanoma, acute leukemia, acoustic neuroma, ampullary carcinoma, anal carcinoma, astrocytoma, basalioma, pancreatic cancer, connective tissue tumor, bladder cancer, bronchial carcinoma, non-small cell bronchial carcinoma, breast cancer, Burkitt's lymphoma, corpus carcinoma, CUP syndrome, colon cancer, cancer of the small intestine, ovarian cancer, endometrial carcinoma, gallbladder cancer, gallbladder carcinomas, uterine cancer, cervical cancer, neck, nose and ear tumors, hematological neoplasia, hairy cell leukemia, urethral cancer, skin cancer, gliomas, testicular cancer, Kaposi's sarcoma, laryngeal cancer, bone cancer, colorectal carcinoma, head/neck tumors, colon carcinoma, craniopharyngeoma, liver cancer, leukemia, lung cancer, non-small cell lung cancer, Hodgkin's lymphoma, non-Hodgkin's lymphoma, stomach cancer, colon cancer, medulloblastoma, melanoma, meningioma, kidney cancer, renal cell carcinomas, oligodendroglioma, esophageal carcinoma, osteolytic carcinomas and osteoplastic carcinomas, osteosarcoma, ovarian carcinoma, pancreatic carcinoma, penile cancer, prostate cancer, tongue cancer, ovary carcinoma, and lymph gland cancer.
41. A kit for diagnosing whether a subject suffering from cancer will be responsive to an albumin-binding chemotherapeutic agent, comprising the metal complex of any one of paragraphs 1-24.
42. Use of a metal complex according to any one of paragraphs 1-24 for the manufacture of a medicament for diagnosing cancer in a subject.
43. Use of a metal complex according to any one of paragraphs 1-24 for the manufacture of a medicament for diagnosing a subject with a cancer responsive to an albumin-binding chemotherapeutic agent.
44. Use of a metal complex according to any one of paragraphs 1-24 for the manufacture of a medicament for assessing the responsiveness of a subject to an albumin-binding chemotherapeutic agent.
45. Use of a metal complex according to any one of paragraphs 1-24 for the manufacture of a medicament for assessing the susceptibility of a cancer in a subject to an albumin-binding chemotherapeutic agent.
46. The use according to any one of paragraphs 44-47, wherein the cancer is selected from adenocarcinoma, uveal melanoma, acute leukemia, acoustic neuroma, ampullary carcinoma, anal carcinoma, astrocytoma, basalioma, pancreatic cancer, connective tissue tumor, bladder cancer, bronchial carcinoma, non-small cell bronchial carcinoma, breast cancer, Burkitt's lymphoma, corpus carcinoma, CUP syndrome, colon cancer, cancer of the small intestine, ovarian cancer, endometrial carcinoma, gallbladder cancer, gallbladder carcinomas, uterine cancer, cervical cancer, neck, nose and ear tumors, hematological neoplasia, hairy cell leukemia, urethral cancer, skin cancer, gliomas, testicular cancer, Kaposi's sarcoma, laryngeal cancer, bone cancer, colorectal carcinoma, head/neck tumors, colon carcinoma, craniopharyngeoma, liver cancer, leukemia, lung cancer, non-small cell lung cancer, Hodgkin's lymphoma, non-Hodgkin's lymphoma, stomach cancer, colon cancer, medulloblastoma, melanoma, meningioma, kidney cancer, renal cell carcinomas, oligodendroglioma, esophageal carcinoma, osteolytic carcinomas and osteoplastic carcinomas, osteosarcoma, ovarian carcinoma, pancreatic carcinoma, penile cancer, prostate cancer, tongue cancer, ovary carcinoma, and lymph gland cancer.
47. A metal complex according to any one of paragraphs 1-24 for use in diagnosing cancer in a subject.
48. A metal complex according to any one of paragraphs 1-24 for use in diagnosing a subject with a cancer responsive to an albumin-binding chemotherapeutic agent.
49. A metal complex according to any one of paragraphs 1-24 for use in assessing the responsiveness of a subject to an albumin-binding chemotherapeutic agent.
50. A metal complex according to any one of paragraphs 1-24 for use in assessing the susceptibility of a cancer in a subject to an albumin-binding chemotherapeutic agent.

A metal complex according to any one of paragraphs 1-24 for use in assessing the ability of an albumin-binding chemotherapeutic agent to treat a cancer in a subject.

1. A metal complex of Formula (I) or (II) or (III): 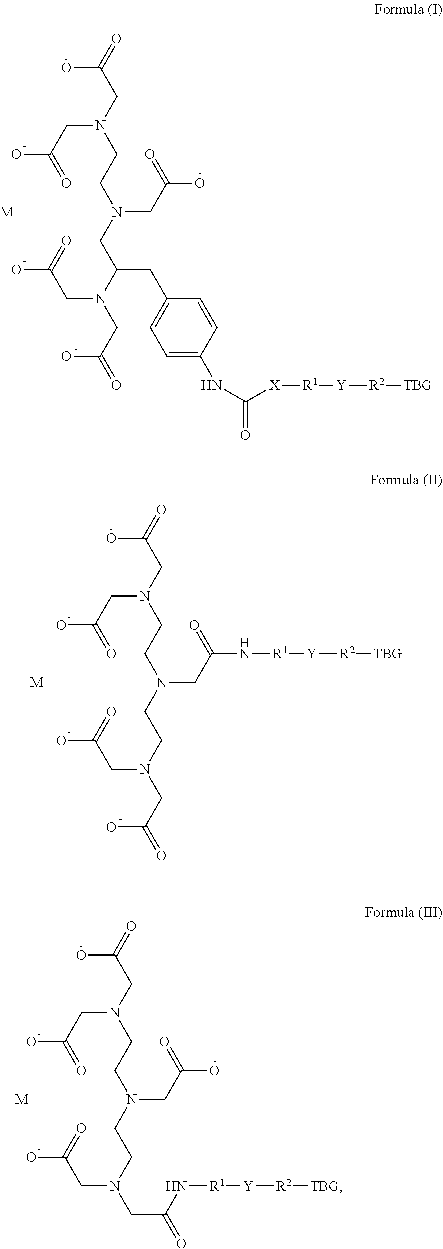
or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
X is absent or selected from —NH—, and —O—;
R¹ is absent or an optionally substituted C₁-C₁₈ alkyl wherein optionally up to six carbon atoms in said C₁-C₁₈ alkyl are each independently replaced with —OCH₂CH₂—;
Y is absent or selected from —O—C(O)—, —C(O)—O—, —NH—C(O)—, —C(O)—NH—, —NH—C(O)—NH—, —O—C(O)—O—, —NH—C(O)—O—, and —O—C(O)—NH—;
R² is an optionally substituted C₁-C₁₈ alkyl wherein optionally up to six carbon atoms in said C₁-C₁₈ alkyl are each independently replaced with —OCH₂CH₂—; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

2. The metal complex of claim 1, wherein said metal complex has the structure of Formula (I): 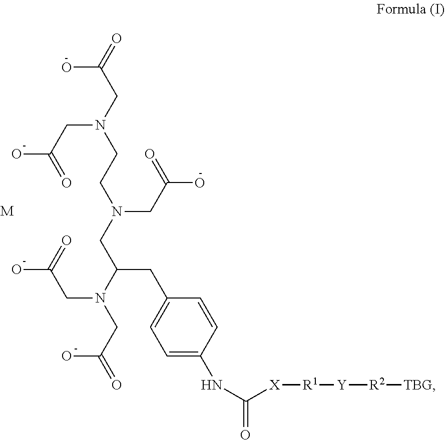
or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
X is absent or selected from —NH—, and —O—;
R¹ is absent or an optionally substituted C₁-C₁₈ alkyl wherein optionally up to six carbon atoms in said C₁-C₁₈ alkyl are each independently replaced with —OCH₂CH₂—;
Y is absent or selected from —O—C(O)—, —C(O)—O—, —NH—C(O)—, —C(O)—NH—, —NH—C(O)—NH—, —O—C(O)—O—, —NH—C(O)—O—, and —O—C(O)—NH—;
R² is an optionally substituted C₁-C₁₈ alkyl wherein optionally up to six carbon atoms in said C₁-C₁₈ alkyl are each independently replaced with —OCH₂CH₂—; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

3. The metal complex of claim 1, wherein said metal complex has the structure of Formula (II): 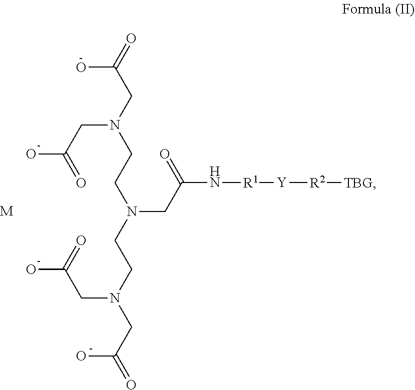
or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
R¹ is absent or an optionally substituted C₁-C₁₈ alkyl wherein optionally up to six carbon atoms in said C₁-C₁₈ alkyl are each independently replaced with —OCH₂CH₂—;
Y is absent or selected from —O—C(O)—, —C(O)—O—, —NH—C(O)—, —C(O)—NH—, —NH—C(O)—NH—, —O—C(O)—O—, —NH—C(O)—O—, and —O—C(O)—NH—;
R² is an optionally substituted C₁-C₁₈ alkyl wherein optionally up to six carbon atoms in said C₁-C₁₈ alkyl are each independently replaced with —OCH₂CH₂—; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

4. A metal complex of claim 1, wherein said metal complex has the structure of Formula (III): 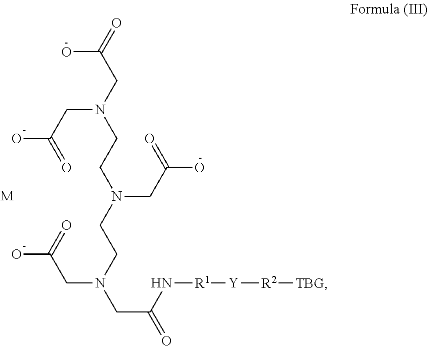
or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
R¹ is absent or an optionally substituted C₁-C₁₈ alkyl wherein optionally up to six carbon atoms in said C₁-C₁₈ alkyl are each independently replaced with —OCH₂CH₂—;
Y is absent or selected from —O—C(O)—, —C(O)—O—, —NH—C(O)—, —C(O)—NH—, —NH—C(O)—NH—, —O—C(O)—O—, —NH—C(O)—O—, and —O—C(O)—NH—;
R² is an optionally substituted C₁-C₁₈ alkyl wherein optionally up to six carbon atoms in said C₁-C₁₈ alkyl are each independently replaced with —OCH₂CH₂—; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

5. The metal complex according to claim 1, wherein said metal complex has the structure of Formula (IV), (V) or (VI): 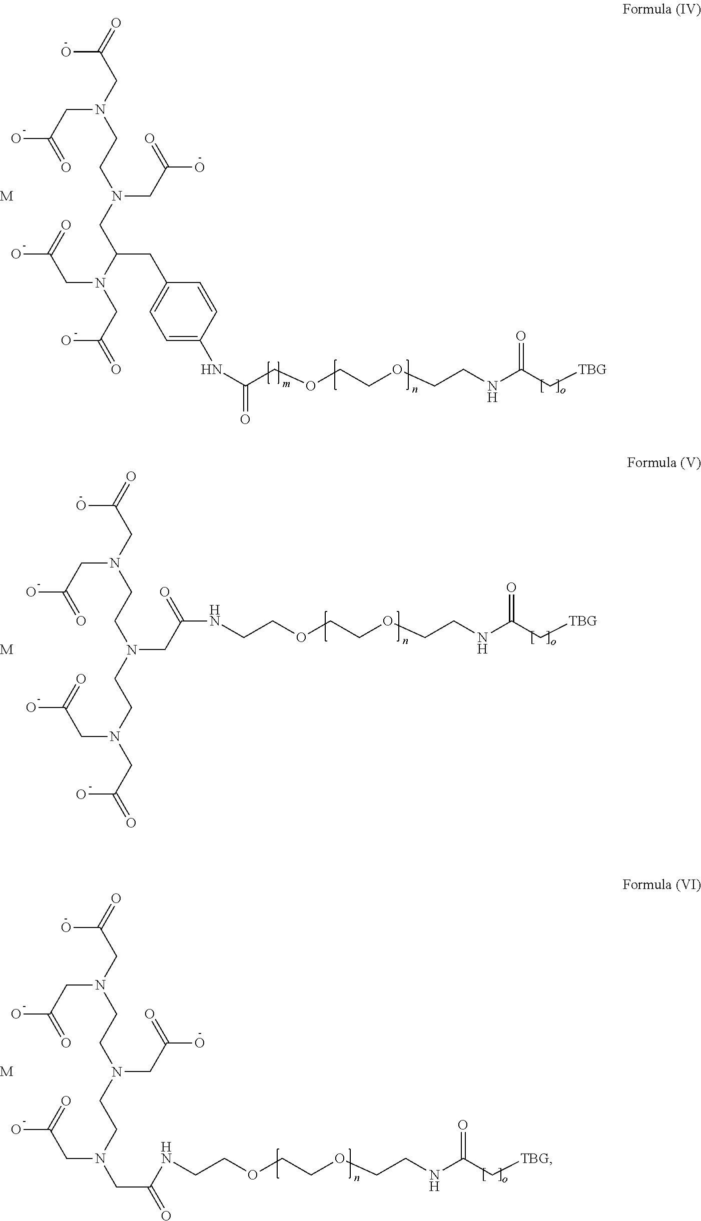
or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
m=1 or 2;
n=1-5;
o=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

6. The metal complex according to claim 5, wherein said metal complex has the structure of Formula (IV): 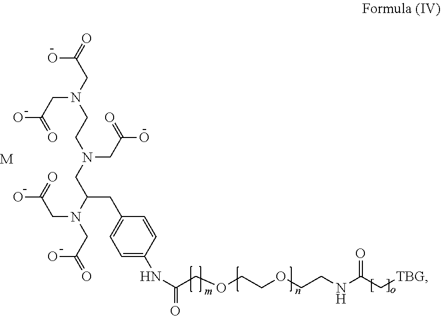
or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
m=1 or 2;
n=1-5;
o=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

7. The metal complex according to claim 5, wherein said metal complex has the structure of Formula (V): 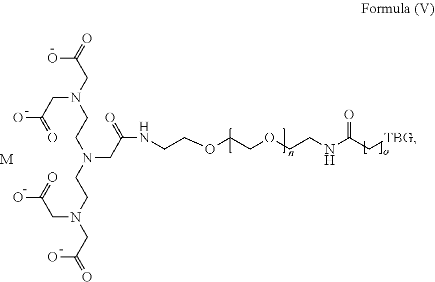
or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
n=1-5;
o=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

8. The metal complex according to claim 5, wherein said metal complex has the structure of Formula (VI): 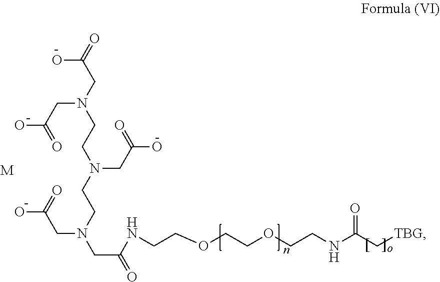
or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
n=1-5;
o=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

9. The metal complex according to claim 1, wherein said metal complex has the structure of Formula (VII), (VIII) or (IX): 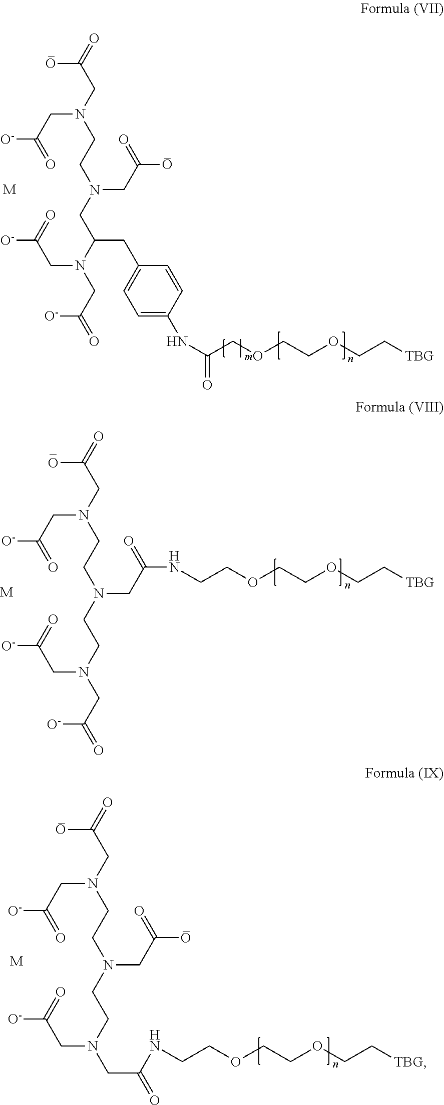
or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
m=1 or 2;
n=1-5; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

10. The metal complex according to claim 9, wherein said metal complex has the structure of Formula (VII): 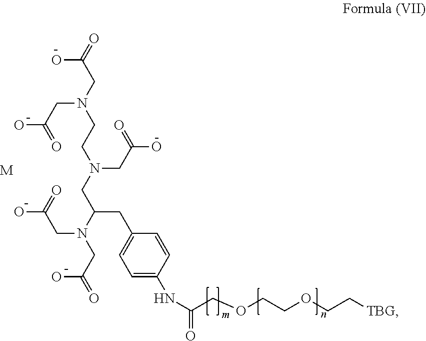
or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
m=1 or 2;
n=1-5; and TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

11. The metal complex according to claim 9, wherein said metal complex has the structure of Formula (VIII): 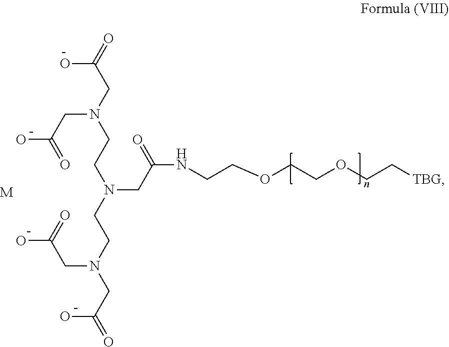
or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
n=1-5; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

12. The metal complex according to claim 9, wherein said metal complex has the structure of Formula (IX): 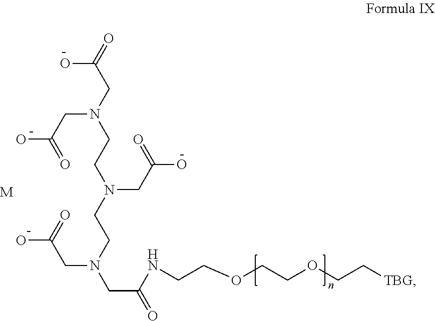
or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
n=1-5; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

13. The metal complex according to claim 1, wherein said metal complex has the structure of Formula (X), (XI) or (XII): 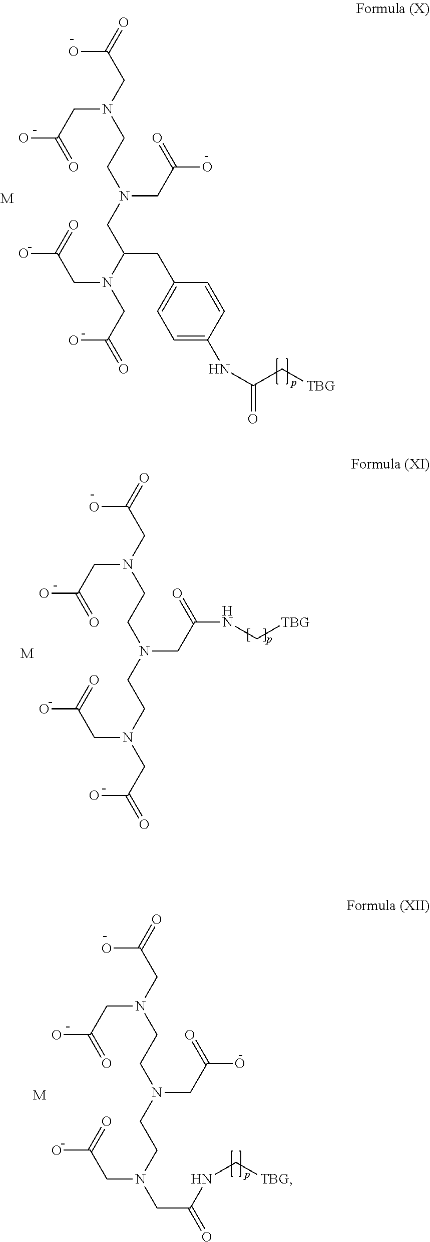
or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
p=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

14. The metal complex according to claim 13, wherein said metal complex has the structure of Formula (X): 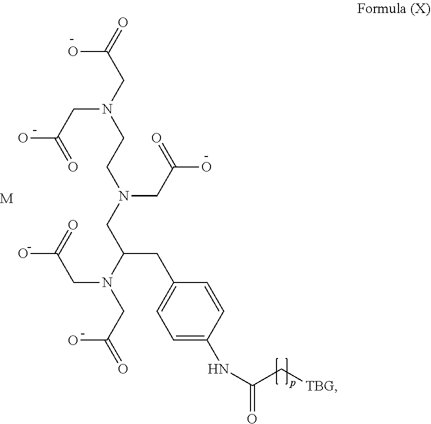
or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
p=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene.

15. The metal complex according to claim 13, wherein said metal complex has the structure of Formula (XI): 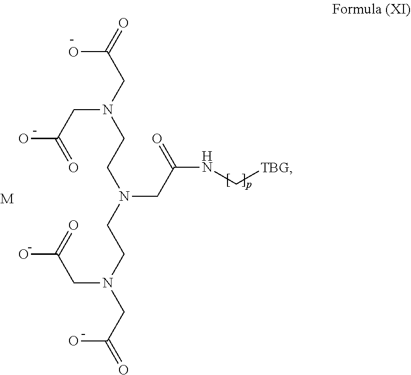
or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
p=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

16. The metal complex according to claim 13, wherein said metal complex has the structure of Formula (XII): 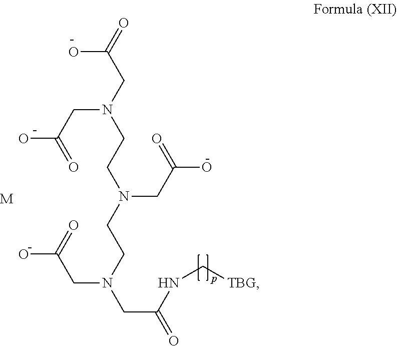
or a conjugate acid thereof, or a pharmaceutically acceptable salt or hydrate thereof;
wherein:
M is ¹¹¹In³⁺, ⁶⁷Ga³⁺, ^{99m}Tc⁴⁺, or ^{99m}Tc³⁺;
p=1-12; and
TBG is a thiol-binding group selected from an optionally substituted maleimide group, an optionally substituted haloacetamide group, an optionally substituted haloacetate group, an optionally substituted pyridyldithio group, an optionally substituted isothiocyanate group, an optionally substituted vinylcarbonyl group, an optionally substituted aziridine group, an optionally substituted disulfide group, and an optionally substituted acetylene group.

17. The metal complex according to claim 1, a conjugate acid thereof, or a pharmaceutically acceptable salt or hydrate thereof, wherein TBG is an optionally substituted maleimide group.

18. The metal complex according to claim 1, a conjugate acid thereof, or a pharmaceutically acceptable salt or hydrate thereof, wherein TBG is

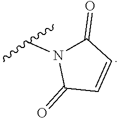 [SMILES: *N1C(=O)C=CC1=O]

19. The metal complex according to claim 1, a conjugate acid thereof, or a pharmaceutically acceptable salt or hydrate thereof, wherein M is ¹¹¹In³⁺.

20. The metal complex according to claim 1, wherein the metal complex is selected from: 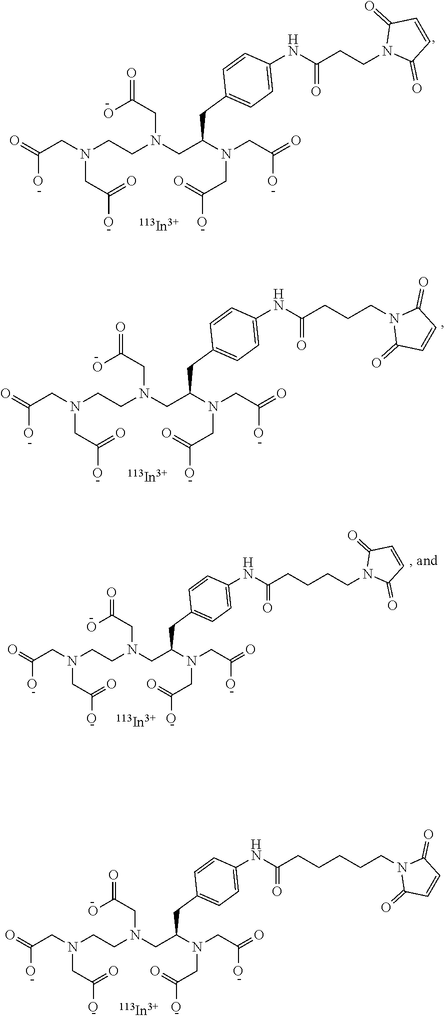
or a conjugate acid thereof, or a pharmaceutically acceptable salt or hydrate thereof.

21. The metal complex according to claim 1, wherein the metal complex is selected from: 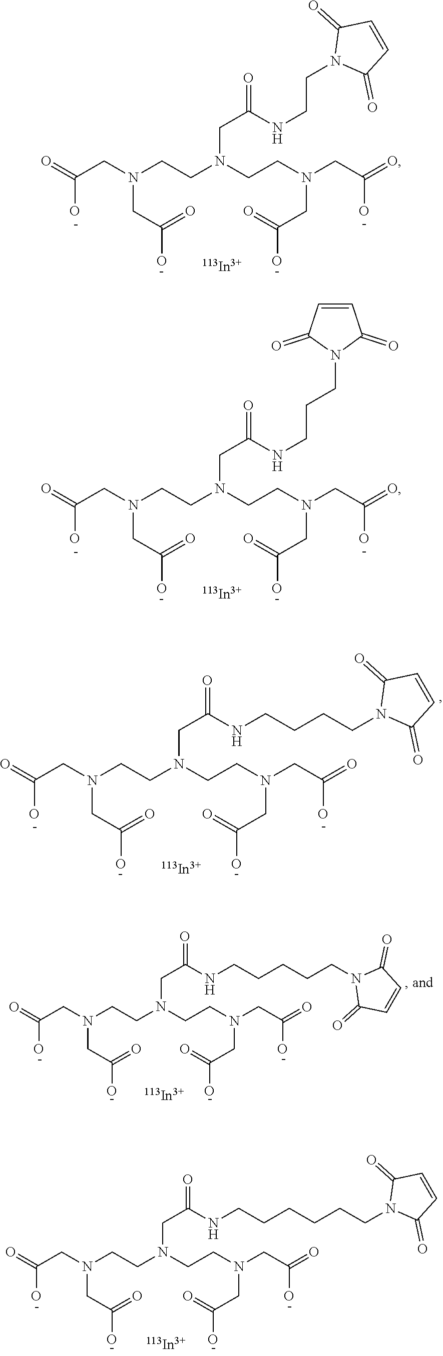
or a conjugate acid thereof, or a pharmaceutically acceptable salt or hydrate thereof.

22. The metal complex according to claim 1, wherein the metal complex is selected 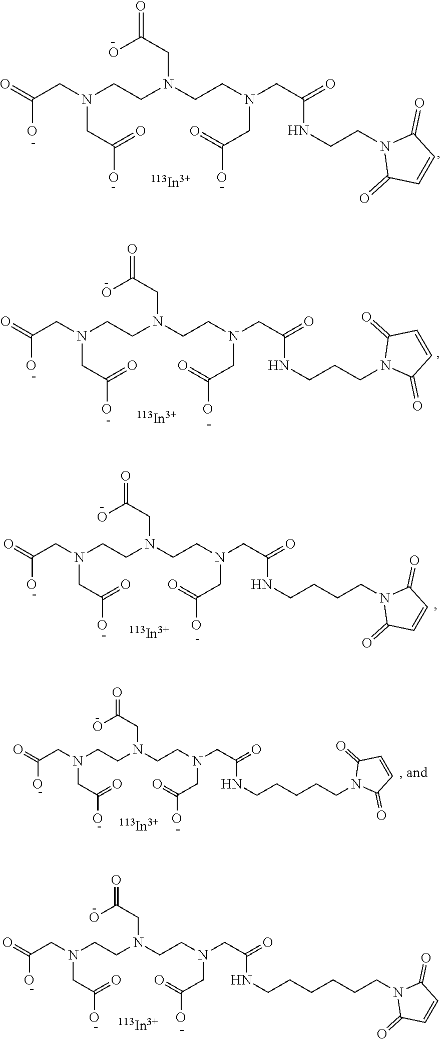
or a conjugate acid thereof, or a pharmaceutically acceptable salt or hydrate thereof.

23. The metal complex according to claim 1, wherein the metal complex is selected from: 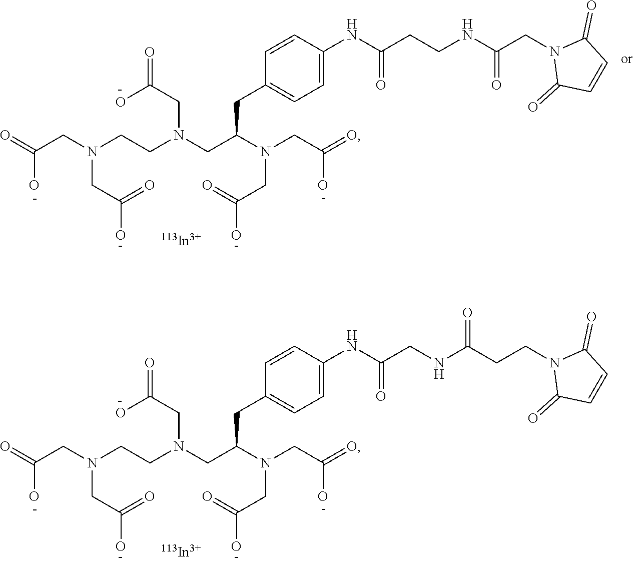
or a conjugate acid thereof, or a pharmaceutically acceptable salt or hydrate thereof.

24. The metal complex according to claim 1, wherein the counter cation of the pharmaceutically acceptable salt is selected from: one or two Na⁺, K⁺, or NH₄⁺; or one Ca²⁺ or Mg²⁺.

25. A pharmaceutical composition comprising a) the metal complex of claim 1, a conjugate acid thereof, or a pharmaceutically acceptable salt or hydrate thereof, and b) a pharmaceutically acceptable carrier.

26. The pharmaceutical composition according to claim 25, wherein the metal complex covalently binds to the thiol group of cysteine-34 of endogenous or exogenous albumin.

27. A method for diagnosing a disease in a subject, wherein said disease is selected from a cancer, a viral disease, autoimmune disease, acute or chronic inflammatory disease, and a disease caused by bacteria, fungi, or other micro-organisms, comprising administering to said subject a diagnostically effective amount of a metal complex according to claim 1, and subsequently performing SPECT (single-photon emission computed tomography) imaging on said subject.

28. The method of claim 27, wherein the disease is cancer.

29. A method of diagnosing cancer in a subject, the method comprising:
administering a detectable amount of the metal complex of claim 1, to the subject;
performing imaging on the subject after administering the metal complex or pharmaceutical composition to detect a signal from a radiolabel of the metal complex, wherein detection of a presence of the signal from the radiolabel in a tissue indicates that the tissue is cancerous, thereby diagnosing cancer in the subject.

30. A method of diagnosing cancer in a subject, the method comprising:
administering a detectable amount of the metal complex of claim 1, to the subject;
performing imaging on the subject after administering the metal complex or pharmaceutical composition, to detect a signal from a radiolabel of the metal complex, wherein detection of a presence of a higher accumulation of the signal from the radiolabel in a tissue in comparison to noncancerous tissue of the same type indicates that the tissue is cancerous, thereby diagnosing cancer in the subject.

31. A method of diagnosing cancer in a subject, the method comprising:
administering to the subject the metal complex of claim 1, wherein the metal complex binds to albumin to form a metal complex-albumin conjugate, wherein the metal complex-albumin conjugate accumulates in cancerous tissue;
performing imaging on the subject after administering the metal complex or pharmaceutical composition, to detect a signal from a radiolabel of the metal complex, wherein detection of a presence of the signal from the radiolabel in the tissue indicates that the tissue is cancerous, thereby diagnosing cancer in the subject.

32. A method of treating cancer in a subject, the method comprising:
administering to the subject the metal complex of claim 1, wherein the metal complex binds to albumin forming a metal complex-albumin conjugate, wherein the metal complex-albumin conjugate accumulates in cancerous tissue;
performing imaging on the subject after administering the metal complex or pharmaceutical composition, to detect a signal from a radiolabel of the metal complex, wherein detection of a presence of the signal from the radiolabel in the tissue indicates that the tissue is cancerous;
diagnosing the subject with cancer in the tissue; and
administering a therapeutically effective amount of a chemotherapeutic agent to the subject.

33. A method of diagnosing a subject with a cancer responsive to an albumin-binding chemotherapeutic agent, the method comprising:
administering to the subject the metal complex of claim 1, wherein the metal complex binds to albumin forming a metal complex-albumin conjugate, wherein the metal complex-albumin conjugate accumulates in cancerous tissue;
performing imaging on the subject after administering the metal complex or pharmaceutical composition, to detect a signal from a radiolabel of the metal complex, wherein detection of a presence of the signal from the radiolabel in the tissue indicates that the tissue is cancerous;
diagnosing the subject with cancer that is responsive to treatment with the albumin-binding chemotherapeutic agent.

34. A method of treating a subject with a cancer responsive to an albumin-binding chemotherapeutic agent, the method comprising:
administering to the subject the metal complex of claim 1, wherein the metal complex binds to albumin forming a metal complex-albumin conjugate, wherein the metal complex-albumin conjugate accumulates in cancerous tissue;
performing imaging on the subject after administering the metal complex or pharmaceutical composition, to detect a signal from a radiolabel of the metal complex, wherein detection a presence of the signal from the radiolabel in the tissue indicates that the tissue is cancerous;
diagnosing the subject with cancer that is responsive to treatment with the albumin-binding chemotherapeutic agent; and
administering a therapeutically effective amount of the albumin-binding chemotherapeutic agent to the subject.

35. A method comprising:
administering to a subject the metal complex of claim 1, wherein the metal complex binds to albumin forming a metal complex-albumin conjugate, wherein the metal complex-albumin conjugate accumulates in cancerous tissue;
performing imaging on the subject after administering the metal complex or pharmaceutical composition, to detect a signal from a radiolabel of the metal complex, wherein detection of a presence of the signal from the radiolabel in the tissue indicates that the tissue is cancerous;
diagnosing the subject with cancer in the tissue; and
administering a therapeutically effective amount of an albumin-binding chemotherapeutic agent to the subject.

36. A method comprising:
administering to a subject the metal complex of claim 1, wherein the metal complex binds to albumin forming a metal complex-albumin conjugate, wherein the metal complex-albumin conjugate accumulates in cancerous tissue;
performing imaging on the subject after administering the metal complex or pharmaceutical composition, to detect a signal from a radiolabel of the metal complex, wherein detection of a presence of a signal from the radiolabel in the tissue indicates that the tissue is cancerous;
diagnosing the subject with cancer in the tissue;
classifying the subject as being responsive to an albumin-binding chemotherapeutic agent; and
administering a therapeutically effective amount of the albumin-binding chemotherapeutic agent to the subject.

37. A method for assessing the responsiveness of a subject having a cancer to an albumin-binding chemotherapeutic agent comprising:
administering to a subject the metal complex of claim 1, wherein the metal complex binds to albumin to form a metal complex-albumin conjugate, wherein the metal complex-albumin conjugate accumulates in cancerous tissue;
performing imaging on the subject after administering the metal complex or pharmaceutical composition, to detect a signal from a radiolabel of the metal complex, wherein detection of a presence of the signal from the radiolabel in the tissue indicates that the tissue is cancerous;
diagnosing the subject with cancer in the tissue; and
classifying the subject as responsive to the albumin-binding chemotherapeutic agent.

38. A method for assessing the susceptibility of a cancer in a subject to an albumin-binding chemotherapeutic agent comprising:
administering to a subject the metal complex of claim 1, wherein the metal complex binds to albumin to form a metal complex-albumin conjugate, wherein the metal complex-albumin conjugate accumulates in cancerous tissue;
performing imaging on the subject after administering the metal complex or pharmaceutical composition, to detect a signal from a radiolabel of the metal complex, wherein detection of a presence of the signal from the radiolabel in the tissue indicates that the tissue is cancerous;
diagnosing the subject with cancer in the tissue; and
classifying the cancer in the subject as susceptible to the albumin-binding chemotherapeutic agent.

39. A method for assessing the ability of an albumin-binding chemotherapeutic agent in treating cancer in a subject comprising:
administering to the subject the metal complex of claim 1, wherein the metal complex binds to albumin to form a metal complex-albumin conjugate, wherein the metal complex-albumin conjugate accumulates in cancerous tissue;
performing imaging on the subject after administering the metal complex or pharmaceutical composition, to detect a signal from a radiolabel of the metal complex, wherein detection of a presence of the signal from the radiolabel in the cancerous tissue indicates that the albumin-binding chemotherapeutic agent is capable of treating the cancer in the subject.

40. The method of claim 27, wherein the metal complex is administered as a metal complex-albumin conjugate formed ex vivo.

41. The method of claim 40, wherein metal complex-albumin conjugate is formed by conjugation of albumin to a moiety corresponding to the TBG of the metal complex; followed by chelation of M.

42. The method of claim 40, wherein metal complex-albumin conjugate is formed by chelation of M to form the metal complex; followed by conjugation of albumin to the TBG of the metal complex to form the metal complex-albumin conjugate.

43. The method of claim 27, wherein the cancer is selected from adenocarcinoma, uveal melanoma, acute leukemia, acoustic neuroma, ampullary carcinoma, anal carcinoma, astrocytoma, basalioma, pancreatic cancer, connective tissue tumor, bladder cancer, bronchial carcinoma, non-small cell bronchial carcinoma, breast cancer, Burkitt's lymphoma, corpus carcinoma, CUP syndrome, colon cancer, cancer of the small intestine, ovarian cancer, endometrial carcinoma, gallbladder cancer, gallbladder carcinomas, uterine cancer, cervical cancer, neck, nose and ear tumors, hematological neoplasia, hairy cell leukemia, urethral cancer, skin cancer, gliomas, testicular cancer, Kaposi's sarcoma, laryngeal cancer, bone cancer, colorectal carcinoma, head/neck tumors, colon carcinoma, craniopharyngeoma, liver cancer, leukemia, lung cancer, non-small cell lung cancer, Hodgkin's lymphoma, non-Hodgkin's lymphoma, stomach cancer, colon cancer, medulloblastoma, melanoma, meningioma, kidney cancer, renal cell carcinomas, oligodendroglioma, esophageal carcinoma, osteolytic carcinomas and osteoplastic carcinomas, osteosarcoma, ovarian carcinoma, pancreatic carcinoma, penile cancer, prostate cancer, tongue cancer, ovary carcinoma, and lymph gland cancer.

44. A kit for diagnosing the responsiveness of a subject suffering from cancer to an albumin-binding chemotherapeutic agent, said kit comprising the metal complex of claim 1.

45.-54. (canceled)
